(19) United States
(12) Patent Application Publication
(10) Pub. No.: US 2015/0292030 A1
(43) Pub. Date: Oct. 15, 2015

(54) METHODS OF CHARACTERIZING AND TREATING MOLECULAR SUBSET OF MUSCLE-INVASIVE BLADDER CANCER

(71) Applicant: BOARD OF REGENTS, THE UNIVERSITY OF TEXAS SYSTEM, Austin,, TX (US)

(72) Inventors: David McConkey, Bellaire, TX (US); Colin Dinney, Houston, TX (US); Bogdan Czerniak, Houston, TX (US); Keith Baggerly, Houston, TX (US); Mai Tran, Houston, TX (US); Woonyoung Choi, Bellaire, TX (US); Neema Navai, Houston, TX (US); Liana Adam, Pearland, TX (US); Arlene Siefker-Radtke, Pearland, TX (US); Matthew Wszolek, Boston, MA (US)

(73) Assignee: BOARD OF REGENTS, THE UNIVERSITY OF TEXAS SYSTEM, Austin, TX (US)

(21) Appl. No.: 14/647,587

(22) PCT Filed: Nov. 27, 2013
(86) PCT No.: PCT/US2013/072349

§ 371 Date: May 27, 2015

Related U.S. Application Data
(60) Provisional application No. 61/730,445, filed on Nov. 27, 2012.

(51) Int. Cl. C12Q 1/68; A61K 33/24; A61K 31/185; C01B 21/092; A61K 31/506; A61K 31/404; A61K 31/53; A61K 31/553; A61K 31/496

(52) U.S. Cl. CPC C12Q 1/6886; A61K 31/553; A61K 33/24; A61K 31/185; A61K 31/496; A61K 31/506; A61K 31/404; A61K 31/53; C01B 21/092; C12Q 2600/178; C12Q 2600/112; C12Q 2600/158; C12Q 2600/106

(57) ABSTRACT

The present application discloses a method of diagnostic testing of primary tumors, circulating tumor cells, serum, and urine to detect high-risk bladder cancers. These results have immediate implications for prognostication and the clinical management of muscle-invasive bladder cancer.

This application claims the benefit of U.S. Provisional Patent Application No. 61/730,445, filed Nov. 27, 2012, the entirety of which is incorporated herein by reference.

The invention was made with government support under Grant No. 2P50CA91846-11 awarded by the National Institutes of Health. The government has certain rights in the invention.

### BACKGROUND OF THE INVENTION

1. Field of the Invention

The present invention relates generally to the fields of cell biology, molecular biology, and cancer. More particularly, it concerns biomarkers for the characterization of bladder cancer subsets.

2. Description of Related Art

Bladder cancer is one of the most common forms of cancer, accounting for more than 70,000 new cases and 14,000 deaths annually in the United States. Bladder cancers progress along two pathways that pose distinct challenges for clinical management. Non-muscle invasive (“superficial”) tumors account for approximately 80% of tumor incidence and are characterized by extremely high rates of recurrence, necessitating extremely expensive long-term clinical follow up. On the other hand, muscle invasive bladder cancers progress rapidly and produce the bulk of patient mortality. Clinically, transurethral resection (TUR) and intravesical therapy are used to manage superficial urothelial cancer, whereas neoadjuvant cisplatin-based chemotherapy followed by radical resection is the standard procedure for muscle invasive tumors. Treatment selection depends heavily on pathologic classification, but current staging systems are imprecise and lead to understaging in a large percentage of cases. Furthermore, cisplatin-based chemotherapy is only effective in 50%-60% of cases, but it is not yet possible to prospectively identify the tumor subset(s) that will be sensitive or resistant to chemotherapy. There is no current method to identify basal and luminal muscle-invasive bladder cancers or to identify lethal subsets of thereof. Thus, there is an urgent need for a more systematic bladder cancer classification system based on a better understanding of the biological mechanisms underlying disease heterogeneity, chemosensitivity, and possible dependency on biological pathways that can be targeted by novel agents.

### SUMMARY OF THE INVENTION

In a first embodiment a method is provided of characterizing a bladder cancer comprising obtaining a sample from a bladder cancer patient and testing to determine the level of expression or activation of a plurality of genes. For example, a method can comprise testing for the expression or activation level of 2, 3, 4, 5, 6, 7, 8, 9 or 10 or more genes. In some aspects, an elevated expression level of miR-200 MAL, FMO9P, BHMT, SNX31, KRT20, SPINK1, DHRS2, UPK2, UPK1A, VSIG2, CD24, CYP2J2, ERBB2, FABP4, FGRF3, FOXA1, GATA3, GPX2, KRT18, KRT19, KRT20, KRT7, KRT8, PPARG or XBP1 compared to a reference level; an elevated activation of AHR; estrogen receptor; MYC; SPDEF; Hdac; SMAD7; PPARA; TRIM24; PPARG; or SREBF2 compared to a reference level; or a decreased activation of TP53; STAT3; SMARCA4; PGR; NFkB; STAT1; HTT; SMAD3; SRF; or MKL1 compared to a reference level indicates that the patient has a luminal bladder cancer (referred to below as a “Cluster 3 bladder cancer”). In further aspects, an elevated expression level of miR-205 CD44, CDH3, KRT1, KRT14, KRT16, KRT5, KRT6A, KRT6B, KRT6C, DSG3, KRT6B, LOC653499, LOC728910, PI3 or S100A7 compared to a reference level; an elevated activation of STAT3; NFkB; IRF7; JUN; STAT1; SP1; TP63; RELA; HIF1A; or IRF3 compared to a reference level; or a decreased activation of estrogen receptor; TRIM24; PPARA; Hdac; GATA3; N-cor; PIAS4; KLF2; SPDEF; or MEOX2 compared to a reference level indicates that the patient has a basal bladder cancer (referred to below as a “Cluster 1 bladder cancer”). In still further aspects, an elevated expression level of one of the ACTG2, CNN1, MYH11, MFAP4, PGM5, FLNC, ACTC1, DES, PCP4, or DMN genes compared to a reference level; an elevated activation of TP53; CDKN2A; RB1; MYOCD; MKL1; TCF3; SMARCB1; SRF; HTT; or Rb compared to a reference level; a decreased activation of TBX2; FOXM1; MYC; SMAD7; E2F2; MYCN; AHR; HEY2; NFE2L2; or SPDEF compared to a reference level; or an elevated or reduced expression level of one or more of the genes as indicated in Table C relative to a reference level indicates that the patient has a p53-like bladder cancer (referred to below as a “Cluster 2 bladder cancer”).

In further aspects, an elevated expression level of one of the AIF1, BCL2, BTLA, CCLS, CD200R1, CD33, CD40, CD8B, CSF1, CTLA4, FASLG, FYB, FYN, HIVEP3, HLA-DRB6, ICAM3, IL10, IL12RB1, IL21R, L4I1, TNFSF14, TRAF1, TRAFD1, VAV1 or ZAP70 genes compared to a reference level indicates that the patient has an immune infiltrating basal bladder cancer. Thus, in some aspects, a method of the embodiments is further defined as a method for identifying an immune infiltrating bladder cancer is a patient.

In yet further embodiment there is provided a method of identifying bladder cancer that has developed chemoresistance comprising obtaining a sample from a bladder cancer patient who has received at least a first chemotherapy and testing the sample to determine the level of expression of one or more of the genes of Table D, wherein an elevated or reduced expression level of one or more of the genes as indicated in Table D relative to a reference level indicates that the patient has a chemoresistant bladder cancer. Thus, in some aspects, a method of treating a bladder cancer patient is provided comprising determining if the patient has developed a bladder cancer that is chemoresistant to a least a first chemotherapy (e.g., cisplatin) in accordance with the embodiments and administering at least a second anti-cancer therapy to the patient (e.g., a chemotherapy or other therapy different from the first chemotherapy).

In still a further embodiment a method is provided of treating a patient having bladder cancer, comprising (a) characterizing the bladder cancer in accordance with embodiments and (b) administering a therapy to the patient based on the characterizing. For example, the treating can comprise administering a FGFR inhibitor therapy to a patient having a luminal bladder cancer; administering an anti-mitotic therapy to a patient having a basal bladder cancer; or administering a therapy that does not comprise cisplatin to a patient having a p53-activated bladder cancer.

In a further embodiment a method is provided of treating a patient having bladder cancer, comprising administering an effective amount of an FGFR inhibitor to a patient determined to have a luminal bladder cancer comprising (a) an elevated expression level ofone or more of miR-200, MAL, FMO9P, BHMT, SNX31, KRT20, SPINK1, DHRS2, UPK2, UPK1A, VSIG2, CD24, CYP2J2, ERBB2, FABP4, FGRF3, FOXA1, GATA3, GPX2, KRT18, KRT19, KRT20, KRT7, KRT8, PPARG or XBP1 genes compared to a reference level; (b) an elevated activation of AHR; estrogen receptor; MYC; SPDEF; Hdac; SMAD7; PPARA; TRIM24; PPARG; or SREBF2 compared to a reference level; or (c) a decreased activation of TP53; STAT3; SMARCA4; PGR; NFkB; STAT1; HTT; SMAD3; SRF; or MKL1 compared to a reference level. For example, in some aspects, the patient was determined to have an elevated level of miR-200 expression (e.g., at least 3-, 4-, or 5-fold higher expression than the reference level). In some aspects, the miR-200 is miR-200c, miR-200a, miR-200b, miR-141, or miR-429.

In a another embodiment a method is provided of treating a patient having bladder cancer, comprising administering an effective amount of an anti-mitotic agent to a patient determined to have a basal bladder cancer comprising (a) an elevated expression level of one or more of miR-205, CD44, CDH3, KRT1, KRT14, KRT16, KRT5, KRT6A, KRT6B, KRT6C, DSG3, KRT6B, LOC653499, LOC728910, PI3 or S100A7 genes compared to a reference level; (b) an elevated activation of STAT3; NFkB; IRF7; JUN; STAT1; SP1; TP63; RELA; HIF1A; or IRF3 compared to a reference level; or (c) a decreased activation of estrogen receptor; TRIM24; PPARA; Hdac; GATA3; N-cor; PIAS4; KLF2; SPDEF; or MEOX2 compared to a reference level. For example, in some aspects, the patient was determined to have an elevated level of miR-205 expression (e.g., at least 1.5- or 2-fold higher expression than the reference level).

In still a further embodiment a method is provided of treating a patient having bladder cancer, comprising administering an effective amount of a non-cisplatin anticancer therapy to a patient determined to have a bladder cancer comprising (a) an elevated expression level of one of the ACTG2, CNN1, MYH11, MFAP4, PGM5, FLNC, ACTC1, DES, PCP4, or DMN genes compared to a reference level; (b) an elevated activation of TP53; CDKN2A; RB1; MYOCD; MKL1; TCF3; SMARCB1; SRF; HTT; or Rb compared to a reference level; (c) a decreased activation of TBX2; FOXM1; MYC; SMAD7; E2F2; MYCN; AHR; HEY2; NFE2L2; or SPDEF compared to a reference level; or (d) an elevated or reduced expression level of one or more of the genes as indicated in Table C relative to a reference level. For example, in some aspects, a method is provided for treating a patient determined to have a bladder cancer comprising an elevated activation of TP53.

In a further embodiment method is provided of characterizing a bladder cancer comprising obtaining a sample from a bladder cancer patient and testing to determine the level of miR-200 or miR-205 in the sample relative to a reference level thereof, wherein an elevated level of miR-200 (e.g., miR-200c, miR-200a, miR-200b, miR-141, or miR-429) relative to the reference is indicative of the bladder cancer being a luminal bladder cancer and an elevated level of miR-205 relative to the reference is indicative of the bladder cancer being a luminal bladder cancer. In certain aspects, the method further comprises identifying the bladder cancer patient as having a luminal bladder cancer if the miR-200 level is determined to be elevated relative to a reference level or a basal bladder cancer if the miR-205 level is determined to be elevated relative to a reference level. For instance, in some cases, the elevated level of miR-200 is defined as an at least 5-fold higher level than the reference level. Likewise, in some cases, an elevated level of miR-205 is defined as an at least 2-fold higher level than the reference level.

In yet a further embodiment, a method is provided of identifying a bladder cancer patient who is a candidate for FGFR inhibitor therapy comprising obtaining a sample from a bladder cancer patient and testing to determine the level of miR-200 in the sample relative to a reference level thereof, wherein an elevated level of miR-200 relative to the reference is indicative of the bladder cancer patient being a candidate for FGFR inhibitor therapy.

In still a further embodiment, a method is provided method of identifying a bladder cancer patient who is a candidate for anti-mitotic therapy comprising obtaining a sample from a bladder cancer patient and testing to determine the level of miR-205 in the sample relative to a reference level thereof, wherein an elevated level of miR-205 relative to the reference is indicative of the bladder cancer patient being a candidate for anti-mitotic therapy.

Thus, in certain aspects, a method of the embodiments comprises identifying a bladder cancer patient as having a luminal bladder cancer, a basal bladder cancer or a p53-activated bladder cancer based on the testing. For example, the identifying can comprise providing a report (e.g., a written, oral or electronic report). In some aspects, a report is provided to the patient, a healthy care payer, a physician, and insurance agent, or an electronic system.

In certain aspects, sample for testing according to the embodiments comprises a sample of the primary tumor (e.g., a biopsy sample). In other aspects, the sample is comprises circulating tumor cell or the contents thereof. For example, the sample can be a serum, or urine sample obtained from the patient.

In certain aspects, a level of expression in the sample is determined using Northern blotting, reverse transcription-quantitative real-time PCR (RT-qPCR), nuclease protection, an in situ hybridization assay, a chip-based expression platform, invader RNA assay platform, or b-DNA detection platform.

Certain aspects of the embodiments concern FGFR inhibitors. For example, the FGFR inhibitor can be a selective FGFR3 inhibitor (e.g., PD173074). Examples, of FGFR inhibitors for use according to embodiments include, without limitation, PKC412; NF449; AZD4547; BGJ398; Dovitinib; TSU-68; BMS-582664; AP24534; PD173074; LY287445; ponatinib; and PD173073.

Certain aspects of the embodiments concern anti-mitotic agents. Examples, of anti-mitotic agents for use according to embodiments include, without limitation, Paclitaxel, Docetaxel, Vinblastine, Vincristine, Vindesine, Vinorelbine, Colchicine, 1,3-diarylpropenone, AZD4877, epothilone B, or cisplatin.

Further methods for characterizing and treating bladder cancer are provided in International Patent Application No. PCT/US2011/026329 and U.S. Publn. 2013/0084241 (each of which is incorporated herein by reference), and may be used in conjunction with the instant methods.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1A-D. (A) Graphs shows Kaplan-Meier disease-specific survival (DSS) curves of 3 subsets based on cluster analysis. (B-C) Graphs shows Kaplan-Meier overall or disease-specific survival (DSS) curves of 3 subsets based on cluster analysis. (D) Graphs show the observed cisplatin resistance of cluster 2 (p53-like bladder cancer cells.

FIG. 2. Transcriptional control of the basal (A and B) and luminal (C and D) subsets. Each panel (A-D) consisted of top and bottom panel. Top: significantly activated/inhibited transcriptional factors after p63 KD in UC14 (A), STAT3 KD in Scaber (B), rosiglitazone treated UC7 (C) and UC9 (D) based on IPA analysis. Bottom: significant changes of basal and luminal markers after p63 KD in UC14 (A), STAT3 KD in Scaber (B), rosiglitazone treated UC7 (C) and UC9 (D).

FIG. 3A-D. Graph shows relative expression p63 in cancers from Cluster 1 versus Clusters 2 & 3.

FIG. 4. Expression of targets of each upstream regulator in three subsets. FIG. 7A: p63 and STAT3 in cluster 1. FIG. 7B: p53 and CDKN2A in cluster 2. FIG. 7C: ER, TRIM24 and PPARγ in cluster 3.

FIG. 5. Basal (top) and luminal (bottom) marker expression in three clusters.

FIG. 6. Proliferation (MTT) assay testing effect of BGJ398 on human bladder cancer cell lines.

FIG. 7. Gene expression profiling-based classification of 16 patient tumors, in each case the fraction of metastases indicated as basel, p53-like or luminal are indicated by the bars (from left to right).

FIG. 8. Epithelial miRNA expression predicts disease specific survival in muscle invasive bladder cancer.

FIG. 9. Graphs showing miRNA expression in the bladder cancer subsets.

FIG. 10. Graphs showing that the disease specific survival of the lethal subset correlates with miR-200c expression.

FIG. 11. ΔNp63α expression in urothelial carcinoma (BC) cells. (A) qRT-PCR quantification of panp63, TAp63 and ΔNp63 mRNA expression in a panel of BC cell lines (n=28). Bars display the relative quantities (RQ) of gene expression ±RQ max and RQ min. (B) Immunoblotting (IB) using the panp63 antibody (4A4, Santa Cruz) to detect all p63 isoforms in wild type cells (n=14) and in TAp63α and ΔNp63α transfected cells.

FIG. 12. ΔNp63α suppresses EMT. (A) Heat map generated from log 2 scale of RQ value (−ΔΔCt) of EMT marker mRNA expressions in BC cell lines (n=28) using Cluster 3.0 and Treeview. (B) Effects of ΔNp63 modulation on cellular morphology. UC6 wild-type (WT) cells were infected with either the empty vector (non-targeting—NT) or with a panp63 shRNA (ΔNp63αKD) containing virus. UC3 wild-type (WT) cells were infected with either the vector control (Vec) or ΔNp63α construct (ΔNp63α) virus. Photos taken under a bright field microscope show changes in cell morphology when ΔNp63α expression is modulated in either cell line. Magnification: 10×. (C) Matrigel invasion assays comparing invasive capacities of UC6 NT versus UC6 ΔNp63αKD and UC3 Vec versus UC3 ΔNp63α cells. Representative images show cells invaded through the Matrigel layers of the transwell inserts. Bars represent mean±SEM from triplicate wells, Student t test, *p<0.05 and **p<0.01.

FIG. 13. ΔNp63α modulates the expression of multiple “epithelial” and “mesenchymal” markers. (A and B) qRT-PCR and IB showing the mRNA and protein expression of p63, ZEB1/2, N-cadherin, Slug, CK-5, CK-14 in ΔNp63α knockdown UC6 (ΔNp63αKD) and ΔNp63α overexpressing UC3 cells. Actin served as an immunoblotting loading control. Bars show the RQ of gene expression ±RQ max and RQ min. * denotes non-specific bands. (C) Flow cytometry analysis results showing the cell surface expression of P-cadherin (upper histogram) and N-cadherin (lower histogram). P-cadherin was labeled with Alexa Fluor 594 and N-cadherin was labeled with allophycocyanin (APC). Statistical analysis demonstrates the mean and median of the fluorescence intensity.

FIG. 14. p63 and miR-205 expression in BC cell lines and BC patients. (A) Heat map generated from log 2 scale of RQ value (−ΔΔCt) of panp63, ΔNp63, pri-miR-205 and mature miR-205 expression in BC cell lines (n=28) using Cluster 3.0 and Treeview. (B) qRT-PCR results for pri-miR-205 and mature miR-205 in cell lines. Bars show the RQ of gene expression ±RQ max and RQ min. (C) Heat map generated from log 2 scale of RQ value (−ΔΔCt) of panp63 and mature miR-205 expression in a cohort of BC patients (n=98) including 32 superficial tumors and 66 muscle invasive tumors. The correlation between p63 and miR-205 is represented in the following graph.

FIG. 15. miR-205 mediates the effects of ΔNp63α on ZEB1/2. (A) qRT-PCR results for pri-miR-205 and mature miR-205 in UC6 ΔNp63αKD and UC3 ΔNp63α overexpressing cells. Bars show the RQ of gene expression ±RQ max and RQ min. (B) qRT-PCR and IB results for ZEB1/2 expression in ΔNp63αKD UC6 cells infected with virus carrying either vector control (ΔNp63αKD/Vec) or miR-205 precursor vector (ΔNp63αKD/miR-205). (C) Diagram depicting the relationship between ΔNp63αKD, miR-205, ZEB1/2 and EMT.

FIG. 16. miR-205HG sequence analysis. Map showing the positions of the p53 response elements (p53REs) and the positions of the primers for the examined regions (Region 1, 2 and 5). The positions were numbered based on the potential transcription start site (TSS) directly 5′ of miR-205 (in red, below) or based on the TSS of the miR-205 host gene (miR-205HG, in black, above). The sequence of the p53RE in region 2 was compared to the consensus p53 binding site in detail. The base that does not correspond to the p53RE consensus sequence is in lowercase.

FIG. 17. ΔNp63α binds to a regulatory region upstream of miR-205 and regulates the transcription of miR-205 and miR-205HG. (A) qRT-PCR results for miR-205HG mRNA expression in ΔNp63αKD UC6 and ΔNp63α-expressing UC3. The Taqman probe for miR-205HG spans the junction of exon 2 and 3. Bars show the RQ of mRNA expression ±RQ max and RQ min. (B) Real time PCR results for miR-205HG and pri-miR-205 expression. Nuclear run-on experiments were used to measure the nascent transcripts generated from miR-205HG and miR-205. HG1 primers were located within exon 1 of miR-205HG. Amplicons generated from Pri1 overlap with the amplicons generated from the Taqman pri-miR-205 primers (ABI). Expression of GAPDH was used as an endogenous control. (C) ChIP results showing that ΔNp63α binds upstream of the miR-205 start site in UC6. Bars represent mean±SD of RQ values for target proteins (IgG, ΔNp63α, and H3) in triplicate samples. Data are representative of two to three independent experiments. (D) ChIP results comparing Pol II binding to target regions in UC6 NT and UC6 ΔNp63αKD cells. RQ values of Pol II binding to regions 1, 2 and 5 were normalized to RQ values of Pol II binding to GAPDH promoter. Bars represent mean±SD of normalized RQ values in triplicate samples. Two-tail, un-paired Student t-test was used to analyze the significance of the difference, *P<0.050, **P<0.01, ***P<0.001.

FIG. 18. High miR-205 expression correlates with poor survival. Kaplan-Meier disease specific survival (DSS) and overall survival (OS) curves generated based on the RT-PCR results of mature miR-205 expression in the primary tumors. (A) DSS and OS of the whole cohort including superficial and muscle-invasive cancers (n=98). High expression of miR-205 was associated with poor probability of DSS and OS (median DSS 13.4 months, median OS 12 months), as compared to lower miR-205 (median DSS>140 months, median OS 69.1 months), log-rank p<0.0001 for DSS and p=0.0004 for OS. (B) DSS and OS for the subset of patients with muscle-invasive cancer (n=66). Patients with elevated miR-205 had worse clinical outcomes (median DSS 8.11 months, median OS 8.11 months) than patients with low miR-205 (median DSS >140 months, median OS 69.1 months).

FIG. 19. Expression of Slug in ΔNp63αKD and overexpressing cells. Slug expression was examined by qRT-PCR. Bars show the RQ of gene expression ±RQ max and RQ min.

FIG. 20. Down regulation of miR-205 and miR-205HG in response to ΔNp63α silencing. (A) qRT-PCR results for pri-miR-205, miR-205 and miR-205HG in ΔNp63αKD BC cell lines. (B) qRT-PCR results shows the expression of miR-205 in ΔNp63 transiently knocked down cells. Bars show the RQ of gene expression ±RQ max and RQ min.

FIG. 21. ΔNp63 binding to region 2 is specific. ChIP result shows that ΔNp63 binding to miR-205 was reduced in ΔNp63αKD UC6. Bars represent mean±SD of RQ values in triplicate samples.

FIG. 22. RNA Pol II binding to miR-205. ChIP result shows a strong enrichment of Pol II binding to region 1. Pol II binding to GAPDH promoter is a positive control. Bars represent mean±SD of RQ values in triplicate samples.

FIG. 23. p53 does not bind to region 2. ChIP results show no significant difference in p53 binding to any region of miR-205 and miR-205HG compared to the IgG negative control. p53 enrichment in the p21 promoter was used as a positive control. Bars represent mean±SD of RQ values in triplicate samples.

FIG. 24. ΔNp63α does not regulate Dicer expression. qRT-PCR results for Dicer mRNA expression in the ΔNp63αKD UC6 and ΔNp63αKD UC14 cells. Bars show the RQ of gene expression ±RQ max and RQ min.

### DESCRIPTION OF ILLUSTRATIVE EMBODIMENTS

### I. The Present Invention

Cancer heterogeneity has a strong influence on patient prognosis and tumor response to conventional and investigational therapies. Muscle-invasive bladder cancers (MIBCs) are a heterogeneous group of tumors that display widely variable clinical outcomes and responses to conventional chemotherapy. The inventors used whole genome mRNA expression profiling (GEP) and unsupervised hierarchical cluster analysis on a cohort of 73 flash frozen primary tumors to characterize the molecular heterogeneity that is present in primary MIBCs. The inventors identified three major “clusters” (subsets) of MIBCs that possess distinct biological properties and then used RNAi in human bladder cancer cell lines to identify key upstream transcriptional regulators that mediated the observed gene expression patterns. The inventors confirmed the existence of the three subsets in a second cohort of 56 formalin-fixed, paraffin-embedded (FFPE) MIBCs and in three other public datasets, and evaluation of clinical outcomes indicated that one of the subsets was associated with poor outcomes. Cluster I, termed the “basal” cluster, contained squamous features and was characterized by active EGFR, ΔNp63, and STAT3 transcription factors and by expression of biomarkers that are found within the basal layer of the normal urothelium (i e., ΔNp63, cytokeratins 5, 6, and 14, and CD44). The basal subset was associated with poor clinical outcomes. Cluster 2 possessed a gene expression signature consistent with p53 activation and appears to be enriched for tumors that are resistant to neoadjuvant cisplatin-based combination chemotherapy. Cluster 3, termed the “luminal” cluster, contained active peroxisome proliferator activator receptor-gamma (PPARγ) and a gene expression signature consistent with active TRIM24 and estrogen receptor-beta (ERβ) and was characterized by expression of biomarkers that are found within the transitional and/or luminal layers of the normal urothelium, including uroplakins, cytokeratin-20, and CD24. The luminal cluster was also enriched for activating mutations in fibroblast growth factor receptor-3 (FGFR3) and the subset of human bladder cancer cell lines that displayed PPARγ pathway activation was selectively sensitive to FGFR inhibitors. ΔNp63 characterized bladder cancers that were sensitive to anti-mitotics, and active PPARγ signaling characterized tumors that were sensitive to FGFR3 inhibitors.

The aggressiveness and metastatic potential of bladder cancer (BC) is heterogenous and largely dependent on grade and stage. At presentation, 55%-60% of tumors are well-differentiated (low grade), and confined to the urothelium or the lamina proria. The majority of these patients can be managed with endoscopic resection and without the need of cystectomy. However, approximately 20% of these patients will ultimately progress to high-grade disease. On the other hand, 40%-45% of patients present with high-grade disease more than half of which have muscle invasion or metastatic disease at presentation. By measuring miRNA-200c expression in 101 patients with bladder cancer (stage I through IV), the inventors have identified a subgroup of muscle-invasive bladder cancer with T2-T4 stage tumors that will die of disease (100%) in less than 3 years after diagnosis. This high-risk group can be easily identified by miRNA measurements in biopsied tumor tissue. Higher than five fold expression values as compared to controls would represent an indication for a more aggressive therapeutic strategy to increase the clinical outcome of these patients.

Using whole genome expression profiling and other molecular biological approaches, the inventors discovered that miR-205 is a direct transcriptional target of ΔNp63 in human bladder cancer cell lines. The inventors used quantitative real-time RT-PCR to measure miR-205 levels in RNA isolated from a cohort of primary bladder cancers and confirmed that high miR-205 was associated with short disease-specific and overall survival. Therefore, miR-205 functions as a biomarker for the lethal basal subset of bladder cancers. Using the same tumor cohort the inventors discovered that miR-200c expression is strongly enriched in the luminal subset of bladder cancers. Analysis of the publicly-available TCGA RNA sequencing data confirmed that miR-200c is expressed almost exclusively by luminal cancers and that the other four members of the miR-200 family are as well. Furthermore, it appears that high miR-200c identifies tumors that are associated with particularly poor outcomes and that the luminal gene expression signature is associated with FGFR inhibitor sensitivity. Since miRNAs are more stable and resistant to degradation than mRNA and they are suitable for analysis in FFPE tissue, plasma, and urine, diagnostic tests that detect the presence of lethal basal and luminal cancers by measuring miR-205 and miR-200 family levels in primary tumors, serum, and/or urine are contemplated.

These findings strongly suggest that molecular subtyping of MIBCs can be used to identify particularly lethal cancers and enrich for tumors that will respond to FGFR inhibitors or neoadjuvant chemotherapy. Therefore, characterization of a given tumor's cluster properties may allow for prospective identification of tumors that will be sensitive to these and other classes of investigational anti-cancer therapies.

### II. Definitions

By “subject” or “patient” is meant any single subject for which therapy is desired, including humans, cattle, dogs, guinea pigs, rabbits, chickens, and so on. Also intended to be included as a subject are any subjects involved in clinical research trials not showing any clinical sign of disease, or subjects involved in epidemiological studies, or subjects used as controls.

“Prognosis” refers to as a prediction of how a patient will progress, and whether there is a chance of recovery. “Cancer prognosis” generally refers to a forecast or prediction of the probable course or outcome of the cancer. As used herein, cancer prognosis includes the forecast or prediction of any one or more of the following: duration of survival of a patient susceptible to or diagnosed with a cancer, duration of recurrence-free survival, duration of progression-free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis and/or cancer progression in a patient susceptible to or diagnosed with a cancer. Prognosis also includes prediction of favorable responses to cancer treatments, such as a conventional cancer therapy.

A good or bad prognosis may, for example, be assessed in terms of patient survival, likelihood of disease recurrence, disease metastasis, or disease progression (patient survival, disease recurrence and metastasis may for example be assessed in relation to a defined time point, e.g. at a given number of years after cancer surgery (e.g. surgery to remove one or more tumors) or after initial diagnosis). In one embodiment, a good or bad prognosis may be assessed in terms of overall survival, disease-free survival or progression-free survival.

In one embodiment, a marker level is compared to a reference level representing the same marker. In certain aspects, the reference level may be a reference level of expression from non-cancerous tissue from the same subject. Alternatively, the reference level may be a reference level of expression from a different subject or group of subjects. For example, the reference level of expression may be an expression level obtained from tissue of a subject or group of subjects without cancer, or an expression level obtained from non-cancerous tissue of a subject or group of subjects with cancer. The reference level may be a single value or may be a range of values. The reference level of expression can be determined using any method known to those of ordinary skill in the art. In some embodiments, the reference level is an average level of expression determined from a cohort of subjects with cancer. The reference level may also be depicted graphically as an area on a graph.

The reference level may comprise data obtained at the same time (e.g., in the same hybridization experiment) as the patient's individual data, or may be a stored value or set of values e.g. stored on a computer, or on computer-readable media. If the latter is used, new patient data for the selected marker(s), obtained from initial or follow-up samples, can be compared to the stored data for the same marker(s) without the need for additional control experiments.

The term “antibody” herein is used in the broadest sense and specifically covers intact monoclonal antibodies, polyclonal antibodies, multispecific antibodies (e.g. bispecific antibodies) formed from at least two intact antibodies, and antibody fragments.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

### III. Biomarkers

The inventors have identified practical cancer prognostic biomarkers and developed methods, systems, and kits to use these markers for cancer prognosis, classification and to guide anti-cancer therapy. In some instances, a cancer may be identified as a basal (cluster 1) bladder cancer. In some aspects, a cancer is determined to be a basal bladder cancer by assaying the expression of genes (e.g., two or more genes) in the cancer. For example, a basal bladder cancer can be a cancer determined to have elevated expression of one, two, three or more of the genes listed in Table A.

TABLE A
Genes with elevated expression in basal (cluster 1) bladder cancers

CD44
CDH3
KRT1
KRT14
KRT16
KRT5
KRT6A
KRT6B
KRT6C

In further aspects, a cancer may be identified as a luminal (cluster 3) bladder cancer. In some aspects, a cancer is determined to be a luminal bladder cancer by assaying the expression of genes (e.g., two or more genes) in the cancer. For example, a luminal bladder cancer can be a cancer determined to have elevated expression of one, two, three or more of the genes listed in Table B.

TABLE B
Genes with elevated expression in
luminal (cluster 3) bladder cancers

CD24
CYP2J2
ERBB2
FABP4
FGFR3
FOXA1
GATA3
GPX2
KRT18
KRT19
KRT20
KRT7
KRT8
PPARG
XBP1

In further aspects, a cancer may be identified as a p53-like (cluster 2) bladder cancer. In some aspects, a cancer is determined to be a p53-like bladder cancer by assaying the expression of genes (e.g., two or more genes) in the cancer. For example, a p53-like bladder cancer can be a cancer determined to have elevated or reduced expression (as compared to a reference level) of one, two, three or more of the genes as indicated in Table C.

TABLE C
Genes with elevated or reduced expression in p53-like (cluster 2) bladder cancers.

| | | Prediction | | |
|---|---|---|---|---|
| | | (based on | | |
| | | expression | | |
| ID | Genes in dataset | direction) | Log Ratio | Findings |
| ILMN_1865764 | ZMAT3 | Activated | 0.586 | Upregulates (6) |
| ILMN_1805828 | VRK1 | Activated | −0.616 | Downregulates (9) |
| ILMN_1714730 | UBE2C | Activated | −1.094 | Downregulates (2) |
| ILMN_1788166 | TTK | Activated | −0.81 | Downregulates (4) |
| ILMN_1748124 | TSC22D3 | Activated | 0.78 | Upregulates (1) |
| ILMN_1796949 | TPX2 | Activated | −0.85 | Downregulates (4) |
| ILMN_1789196 | TPM2 | Activated | 1.553 | Upregulates (6) |
| ILMN_1661717 | TFDP1 | Activated | −0.768 | Downregulates (1) |
| ILMN_1744795 | TBL1X | Activated | 0.745 | Upregulates (1) |
| ILMN_1745593 | STMN1 | Activated | −0.664 | Downregulates (7) |
| ILMN_1749792 | SORBS1 | Activated | 1.781 | Upregulates (4) |
| ILMN_1748923 | SMC2 | Activated | −0.673 | Downregulates (3) |
| ILMN_1678669 | RRM2 | Activated | −1.003 | Downregulates (2) |
| ILMN_1658143 | RFC3 | Activated | −0.599 | Downregulates (1) |
| ILMN_2210129 | PRIM1 | Activated | −0.732 | Downregulates (2) |
| ILMN_1728934 | PRC1 (includes EG:233406) | Activated | −0.983 | Downregulates (11) |
| ILMN_1774336 | POLE2 | Activated | −0.678 | Downregulates (1) |
| ILMN_1675331 | PEG3 | Activated | 1.181 | Upregulates (0) |
| ILMN_2086470 | PDGFRA | Activated | 0.963 | Upregulates (1) |
| ILMN_1729161 | NOTCH1 | Activated | 0.806 | Upregulates (19) |
| ILMN_1664511 | NDC80 | Activated | −0.669 | Downregulates (2) |
| ILMN_2193325 | MMP23B | Activated | 1.367 | Upregulates (2) |
| ILMN_1704702 | MCM7 | Activated | −0.646 | Downregulates (2) |
| ILMN_2412860 | MCM4 | Activated | −0.715 | Downregulates (3) |
| ILMN_1777564 | MAD2L1 | Activated | −1.328 | Downregulates (3) |
| ILMN_1651254 | LPP | Activated | 1.09 | Upregulates (2) |
| ILMN_1811472 | KIF23 | Activated | −0.76 | Downregulates (3) |
| ILMN_2285996 | KIAA0101 | Activated | −1.067 | Downregulates (5) |
| ILMN_2297765 | KCNMA1 | Activated | 1.305 | Upregulates (1) |
| ILMN_2132982 | IGFBP5 | Activated | 1.697 | Upregulates (2) |
| ILMN_2413084 | HSPA8 | Activated | −0.594 | Downregulates (2) |
| ILMN_1781942 | HMMR | Activated | −0.954 | Downregulates (1) |
| ILMN_2200331 | H2AFX | Activated | −0.597 | Downregulates (2) |
| ILMN_1748797 | GRB2 | Activated | −0.62 | Downregulates (1) |
| ILMN_1726666 | GPX3 | Activated | 1.034 | Upregulates (1) |
| ILMN_1805842 | FHL1 (includes EG:14199) | Activated | 1.657 | Upregulates (3) |
| ILMN_1695290 | FERMT2 | Activated | 1.373 | Upregulates (3) |
| ILMN_1755834 | FEN1 | Activated | −0.798 | Downregulates (3) |
| ILMN_1652913 | EZH2 | Activated | −0.813 | Downregulates (1) |
| ILMN_1677200 | CYFIP2 | Activated | 0.924 | Upregulates (1) |
| ILMN_1791447 | CXCL12 (includes EG:20315) | Activated | 1.193 | Upregulates (2) |
| ILMN_1654072 | CX3CL1 | Activated | 1.075 | Upregulates (4) |
| ILMN_1804955 | CTSF | Activated | 0.609 | Upregulates (1) |
| ILMN_1811921 | CSRP1 | Activated | 1.13 | Upregulates (3) |
| ILMN_1729216 | CRYAB | Activated | 1.294 | Upregulates (2) |
| ILMN_1786598 | COL14A1 | Activated | 0.812 | Upregulates (3) |
| ILMN_1810054 | CNN1 (includes EG:1264) | Activated | 2.147 | Upregulates (3) |
| ILMN_1719256 | CKS1B | Activated | −0.822 | Downregulates (1) |
| ILMN_1664630 | CHEK1 | Activated | −0.759 | Downregulates (10) |
| ILMN_1747911 | CDK1 | Activated | −1.011 | Downregulates (18) |
| ILMN_2412384 | CCNE2 | Activated | −0.896 | Downregulates (1) |
| ILMN_2067656 | CCND2 | Activated | 1.069 | Upregulates (1) |
| ILMN_1786125 | CCNA2 | Activated | −1.099 | Downregulates (4) |
| ILMN_2202948 | BUB1 (includes EG:100307076) | Activated | −0.863 | Downregulates (4) |
| ILMN_1684217 | AURKB | Activated | −0.925 | Downregulates (3) |
| ILMN_1680955 | AURKA | Activated | −0.89 | Downregulates (3) |
| ILMN_1671703 | ACTA2 | Activated | 1.636 | Upregulates (4) |
| ILMN_1701461 | TIMP3 | Inhibited | 1.274 | Downregulates (9) |
| ILMN_2083946 | TGFA | Inhibited | −1.008 | Upregulates (8) |
| ILMN_1673676 | SNX5 | Inhibited | −0.858 | Upregulates (0) |
| ILMN_2370365 | RFC4 | Inhibited | −0.905 | Upregulates (1) |
| ILMN_1753196 | PTTG1 | Inhibited | −0.927 | Upregulates (5) |
| ILMN_2383611 | PTPRE | Inhibited | −0.703 | Upregulates (1) |
| ILMN_1748831 | PPP1R13B | Inhibited | 0.785 | Downregulates (1) |
| ILMN_1706958 | PCNA | Inhibited | −0.615 | Upregulates (21) |
| ILMN_2404688 | NUPR1 | Inhibited | 1.091 | Downregulates (1) |
| ILMN_1713875 | NME1 | Inhibited | −0.603 | Upregulates (1) |
| ILMN_1756806 | MCL1 | Inhibited | −0.861 | Upregulates (4) |
| ILMN_2184373 | IL8 | Inhibited | −1.795 | Upregulates (1) |
| ILMN_2056087 | IGF1 | Inhibited | 0.706 | Downregulates (9) |
| ILMN_1674411 | CKAP2 | Inhibited | −0.7 | Upregulates (8) |
| ILMN_1790100 | C11orf82 | Inhibited | −0.867 | Upregulates (6) |
| ILMN_2095610 | ANXA8/ANXA8L1 | Inhibited | −1.183 | Upregulates (2) |
| ILMN_1711899 | ANXA2 | Inhibited | −1.084 | Upregulates (1) |
| ILMN_1739645 | ANLN | Affected | −0.788 | Regulates (0) |
| ILMN_1815184 | ASPM | Affected | −0.952 | Regulates (1) |
| ILMN_2048700 | ATAD2 | Affected | −0.885 | Regulates (1) |
| ILMN_1725139 | CA9 | Affected | −1.336 | Regulates (1) |
| ILMN_1801939 | CCNB2 | Affected | −1 | Regulates (3) |
| ILMN_2384785 | CCNE1 | Affected | −0.92 | Regulates (1) |
| ILMN_1666305 | CDKN3 | Affected | −0.819 | Regulates (0) |
| ILMN_1749829 | DLGAP5 | Affected | −0.853 | Regulates (1) |
| ILMN_1673721 | EXO1 (includes EG:26909) | Affected | −0.676 | Regulates (2) |
| ILMN_1792323 | HDC | Affected | 0.877 | Regulates (1) |
| ILMN_1813295 | LMO3 | Affected | 1.343 | Regulates (2) |
| ILMN_1666713 | LYPLA1 | Affected | −0.726 | Regulates (1) |
| ILMN_1694240 | MAP2K1 | Affected | −0.618 | Regulates (4) |
| ILMN_1658015 | MBNL2 | Affected | 0.67 | Regulates (1) |
| ILMN_1769299 | MTMR11 | Affected | 0.767 | Regulates (1) |
| ILMN_2409298 | NUSAP1 | Affected | −0.919 | Regulates (1) |
| ILMN_1760303 | PIK3R1 | Affected | 0.803 | Regulates (1) |
| ILMN_1698323 | PLEKHB2 | Affected | −0.686 | Regulates (1) |
| ILMN_1695827 | PPP1CA | Affected | −0.628 | Regulates (3) |
| ILMN_1785891 | PRKD1 | Affected | 0.851 | Regulates (4) |
| ILMN_2077550 | RACGAP1 | Affected | −0.751 | Regulates (1) |
| ILMN_1670353 | RAD51AP1 | Affected | −0.915 | Regulates (1) |
| ILMN_1670305 | SERPING1 | Affected | 0.981 | Regulates (1) |
| ILMN_1711470 | UBE2T | Affected | −0.715 | Regulates (1) |
| ILMN_1786065 | UHRF1 | Affected | −1.183 | Regulates (1) |

In further aspects, methods are provided for identifying cancer that have developed chemotherapy resistance (e.g., basal or luminal cancer that have been treated with a chemotherapeutic and have developed resistance). In some aspects, a cancer is determined to be a chemoresistant bladder cancer by assaying the expression of genes (e.g., two or more genes) in the cancer. For example, a a chemoresistant bladder cancer can be a cancer determined to have elevated or reduced expression (as compared to a reference level) of one, two, three or more of the genes as indicated in Table D.

TABLE D
Genes with elevated or reduced expression in a chemoresistant bladder cancer.

| | | Prediction | | |
|---|---|---|---|---|
| | | (based on | | |
| | | expression | | |
| ID | Genes in dataset | direction) | Log Ratio | Findings |
| ILMN_1651237 | CDT1 | Activated | −0.761 | Downregulates (1) |
| ILMN_1653443 | CDK2 | Activated | −0.811 | Downregulates (1) |
| ILMN_1655906 | FBXW7 | Affected | 0.824 | Regulates (2) |
| ILMN_1657796 | STMN1 | Activated | −1.089 | Downregulates (7) |
| ILMN_1658143 | RFC3 | Activated | −0.599 | Downregulates (1) |
| ILMN_1659350 | CASP6 | Inhibited | −1.000 | Upregulates (4) |
| ILMN_1661196 | CSF2RA | Activated | 0.816 | Upregulates (1) |
| ILMN_1661599 | DDIT4 | Activated | 0.595 | Upregulates (5) |
| ILMN_1661674 | VCL | Activated | 0.888 | Upregulates (1) |
| ILMN_1664516 | CENPF | Activated | −1.252 | Downregulates (1) |
| ILMN_1671250 | CLIC4 | Activated | 0.774 | Upregulates (10) |
| ILMN_1671843 | PSRC1 | Inhibited | −1.120 | Upregulates (8) |
| ILMN_1672486 | TCF7L2 | Inhibited | 0.595 | Downregulates (2) |
| ILMN_1673522 | MOCOS | Affected | −0.916 | Regulates (3) |
| ILMN_1673673 | PBK | Activated | −1.434 | Downregulates (2) |
| ILMN_1676984 | DDIT3 | Activated | 0.766 | Upregulates (2) |
| ILMN_1678535 | ESR1 | Activated | 1.803 | Upregulates (6) |
| ILMN_1678962 | DFFB | Inhibited | −0.971 | Upregulates (1) |
| ILMN_1679476 | GART | Inhibited | −0.690 | Upregulates (1) |
| ILMN_1680618 | MYC | Inhibited | 1.646 | Downregulates (19) |
| ILMN_1680955 | AURKA | Activated | −1.737 | Downregulates (3) |
| ILMN_1681503 | MCM2 | Activated | −2.000 | Downregulates (2) |
| ILMN_1683441 | NCAPD3 | Affected | −1.059 | Regulates (1) |
| ILMN_1686116 | THBS1 | Activated | 0.880 | Upregulates (7) |
| ILMN_1686535 | SLC37A3 | Affected | −0.862 | Regulates (1) |
| ILMN_1686846 | AKAP12 | Inhibited | 1.098 | Downregulates (1) |
| ILMN_1690822 | VAPA | Activated | 0.669 | Upregulates (1) |
| ILMN_1691180 | OTX1 | Inhibited | −1.599 | Upregulates (4) |
| ILMN_1691433 | PIK3R1 | Affected | 0.696 | Regulates (1) |
| ILMN_1692080 | ANKH | Affected | 1.333 | Regulates (1) |
| ILMN_1693060 | VEGFA | Inhibited | 1.163 | Downregulates (19) |
| ILMN_1694126 | KIF24 | Affected | −1.644 | Regulates (3) |
| ILMN_1695382 | TSC22D3 | Activated | 1.915 | Upregulates (1) |
| ILMN_1695414 | ASF1B | Affected | −0.943 | Regulates (1) |
| ILMN_1695509 | PTPN12 | Affected | 0.816 | Regulates (4) |
| ILMN_1696360 | CTSB | Inhibited | 0.799 | Downregulates (1) |
| ILMN_1696591 | RB1 | Inhibited | −0.837 | Upregulates (7) |
| ILMN_1699737 | TRAP1 | Activated | −0.811 | Downregulates (1) |
| ILMN_1701114 | GBP1 | Activated | 1.043 | Upregulates (1) |
| ILMN_1701120 | BCL2 | Inhibited | 1.316 | Downregulates (57) |
| ILMN_1701402 | IKBIP | Affected | 0.604 | Regulates (1) |
| ILMN_1701731 | AKR1B1 | Inhibited | −0.599 | Upregulates (1) |
| ILMN_1703906 | HJURP | Affected | −0.916 | Regulates (1) |
| ILMN_1707649 | MPDZ | Affected | 0.888 | Regulates (1) |
| ILMN_1707858 | H2AFZ | Affected | −0.862 | Regulates (1) |
| ILMN_1708416 | ARL6IP1 | Activated | −0.690 | Downregulates (1) |
| ILMN_1709613 | IGF1 | Inhibited | 2.198 | Downregulates (9) |
| ILMN_1710937 | IFI16 | Activated | 0.714 | Upregulates (1) |
| ILMN_1711005 | CDC25A | Activated | −1.474 | Downregulates (4) |
| ILMN_1711470 | UBE2T | Affected | −1.152 | Regulates (1) |
| ILMN_1711748 | PLTP | Activated | 0.614 | Upregulates (1) |
| ILMN_1712639 | AIFM2 | Inhibited | −1.089 | Upregulates (3) |
| ILMN_1713603 | PRKCB | Affected | 1.091 | Regulates (4) |
| ILMN_1714383 | TPD52L1 | Affected | 1.345 | Regulates (2) |
| ILMN_1714730 | UBE2C | Activated | −1.152 | Downregulates (2) |
| ILMN_1714738 | SCMH1 | Affected | 0.687 | Regulates (1) |
| ILMN_1716218 | RPS6KA2 | Activated | 0.880 | Upregulates (2) |
| ILMN_1716224 | STARD4 | Activated | 1.098 | Upregulates (1) |
| ILMN_1716651 | RUNX2 | Inhibited | 0.949 | Downregulates (2) |
| ILMN_1719616 | DNASE1 | Activated | −1.943 | Downregulates (1) |
| ILMN_1720114 | GMNN | Affected | −0.621 | Regulates (1) |
| ILMN_1720829 | ZFP36 | Activated | 1.570 | Upregulates (1) |
| ILMN_1720965 | TULP4 | Affected | −1.000 | Regulates (1) |
| ILMN_1722127 | RAD54B | Activated | −1.358 | Downregulates (1) |
| ILMN_1722781 | EGR3 | Inhibited | 1.934 | Downregulates (1) |
| ILMN_1722838 | MRPL46 | Affected | −0.761 | Regulates (3) |
| ILMN_1724489 | RFC4 | Inhibited | −1.434 | Upregulates (1) |
| ILMN_1725193 | IGFBP2 | Affected | 0.791 | Regulates (5) |
| ILMN_1726496 | SEL1L | Inhibited | −1.059 | Upregulates (1) |
| ILMN_1727080 | MYO6 | Inhibited | −0.690 | Upregulates (10) |
| ILMN_1727762 | CASP1 | Activated | 0.740 | Upregulates (5) |
| ILMN_1730084 | COMT | Affected | −0.644 | Regulates (1) |
| ILMN_1731720 | PDRG1 | Activated | −1.218 | Downregulates (2) |
| ILMN_1732516 | KNTC1 | Affected | −0.889 | Regulates (1) |
| ILMN_1740842 | SALL2 | Activated | −1.059 | Downregulates (1) |
| ILMN_1742044 | GNAI1 | Activated | 1.350 | Upregulates (1) |
| ILMN_1742145 | ESPL1 | Affected | −1.000 | Regulates (1) |
| ILMN_1742866 | F2R | Activated | 0.926 | Upregulates (1) |
| ILMN_1744862 | TGFBR2 | Inhibited | 0.632 | Downregulates (2) |
| ILMN_1745154 | PARD6B | Affected | −1.252 | Regulates (2) |
| ILMN_1745927 | TGFBR1 | Inhibited | 0.782 | Downregulates (1) |
| ILMN_1748908 | PROSC | Affected | −0.889 | Regulates (1) |
| ILMN_1751444 | NCAPG | Activated | −0.943 | Downregulates (1) |
| ILMN_1751464 | TNFSF9 | Activated | 1.111 | Upregulates (3) |
| ILMN_1756043 | WDHD1 | Activated | −1.152 | Downregulates (2) |
| ILMN_1756999 | RBL2 | Affected | 0.595 | Regulates (1) |
| ILMN_1757437 | UMPS | Activated | −1.184 | Downregulates (1) |
| ILMN_1758906 | GNA13 | Affected | 0.642 | Regulates (2) |
| ILMN_1759250 | TAP2 | Inhibited | 0.687 | Downregulates (1) |
| ILMN_1760858 | RAB8A | Inhibited | −0.889 | Upregulates (1) |
| ILMN_1762003 | SEC62 | Affected | 0.642 | Regulates (2) |
| ILMN_1762766 | PTPRA | Activated | −0.737 | Downregulates (5) |
| ILMN_1763386 | BID | Inhibited | −1.029 | Upregulates (14) |
| ILMN_1768260 | GAS6 | Activated | 1.531 | Upregulates (2) |
| ILMN_1769245 | GLIPR1 | Activated | 1.417 | Upregulates (10) |
| ILMN_1769406 | PIAS2 | Activated | 1.021 | Upregulates (1) |
| ILMN_1771039 | GTSE1 | Inhibited | −1.059 | Upregulates (4) |
| ILMN_1772910 | GAS1 | Affected | 1.536 | Regulates (1) |
| ILMN_1776953 | MYL9 | Activated | 0.824 | Upregulates (3) |
| ILMN_1778152 | FIGNL1 | Affected | −1.286 | Regulates (1) |
| ILMN_1778444 | FKBP5 | Activated | 1.029 | Upregulates (2) |
| ILMN_1779965 | AK1 | Activated | 0.660 | Upregulates (9) |
| ILMN_1781207 | FYN | Affected | 0.949 | Regulates (4) |
| ILMN_1781285 | DUSP1 | Activated | 0.903 | Upregulates (4) |
| ILMN_1783170 | ING3 | Affected | 0.748 | Regulates (1) |
| ILMN_1783497 | PANK1 | Inhibited | −1.089 | Upregulates (10) |
| ILMN_1785402 | LTBP1 | Activated | 1.930 | Upregulates (2) |
| ILMN_1790100 | C11orf82 | Inhibited | −0.971 | Upregulates (6) |
| ILMN_1791346 | ATF3 | Activated | 2.692 | Upregulates (8) |
| ILMN_1793522 | PRKAB1 | Inhibited | −1.152 | Upregulates (10) |
| ILMN_1793849 | TANK | Inhibited | 1.077 | Downregulates (1) |
| ILMN_1795852 | CCNE1 | Affected | −1.089 | Regulates (1) |
| ILMN_1796417 | ASNS | Affected | −0.621 | Regulates (18) |
| ILMN_1797236 | TGM2 | Activated | 0.722 | Upregulates (1) |
| ILMN_1799139 | PLOD2 | Inhibited | 0.660 | Downregulates (1) |
| ILMN_1800512 | HMOX1 | Inhibited | −0.786 | Upregulates (7) |
| ILMN_1800975 | PSME3 | Activated | −1.184 | Downregulates (1) |
| ILMN_1801939 | CCNB2 | Affected | −0.916 | Regulates (3) |
| ILMN_1803686 | ADA | Affected | −0.599 | Regulates (2) |
| ILMN_1805737 | PFKP | Activated | −0.621 | Downregulates (2) |
| ILMN_1805828 | VRK1 | Activated | −0.690 | Downregulates (9) |
| ILMN_1805842 | FHL1 | Activated | 1.257 | Upregulates (3) |
| ILMN_1805990 | BAK1 | Inhibited | −0.644 | Upregulates (8) |
| ILMN_1806790 | ROBO1 | Activated | 1.029 | Upregulates (1) |
| ILMN_1808132 | FAS | Activated | 0.926 | Upregulates (67) |
| ILMN_1808391 | DUSP4 | Affected | 1.091 | Regulates (4) |
| ILMN_1811472 | KIF23 | Activated | −0.916 | Downregulates (3) |
| ILMN_1813489 | RAF1 | Affected | −0.786 | Regulates (4) |
| ILMN_1814327 | AGTR1 | Activated | 1.077 | Upregulates (4) |
| ILMN_2041046 | CKS1B | Activated | −1.152 | Downregulates (1) |
| ILMN_2062468 | IGFBP7 | Affected | 0.766 | Regulates (2) |
| ILMN_2067656 | CCND2 | Activated | 0.740 | Upregulates (1) |
| ILMN_2077550 | RACGAP1 | Affected | −1.322 | Regulates (1) |
| ILMN_2105919 | FGF2 | Affected | 0.774 | Regulates (5) |
| ILMN_2111323 | PDCD6IP | Affected | −0.761 | Regulates (7) |
| ILMN_2112460 | MAD2L1 | Inhibited | 0.604 | Downregulates (3) |
| ILMN_2137789 | KLF4 | Activated | 1.642 | Upregulates (2) |
| ILMN_2154654 | PTP4A1 | Inhibited | 0.807 | Downregulates (2) |
| ILMN_2157957 | GTF2H1 | Affected | 0.623 | Regulates (1) |
| ILMN_2170595 | RRM2B | Activated | 0.895 | Upregulates (12) |
| ILMN_2188264 | CYR61 | Affected | 1.233 | Regulates (1) |
| ILMN_2193325 | MMP23B | Activated | 1.541 | Upregulates (2) |
| ILMN_2196328 | POSTN | Affected | 1.379 | Regulates (4) |
| ILMN_2201668 | SLC19A2 | Affected | 0.986 | Regulates (3) |
| ILMN_2212909 | MELK | Activated | −1.152 | Downregulates (1) |
| ILMN_2228732 | CCNG2 | Inhibited | −1.089 | Upregulates (2) |
| ILMN_2261882 | KIAA0368 | Activated | −1.184 | Downregulates (1) |
| ILMN_2266224 | SORBS1 | Activated | 1.618 | Upregulates (4) |
| ILMN_2269977 | GLUL | Activated | 1.884 | Upregulates (1) |
| ILMN_2285480 | LBR | Activated | −0.889 | Downregulates (1) |
| ILMN_2294644 | RFWD2 | Activated | 0.911 | Upregulates (3) |
| ILMN_2294784 | PRDM1 | Activated | 1.551 | Upregulates (1) |
| ILMN_2297765 | KCNMA1 | Activated | 0.872 | Upregulates (1) |
| ILMN_2323172 | CSF3R | Activated | 1.084 | Upregulates (1) |
| ILMN_2329744 | PMS2 | Affected | −0.889 | Regulates (3) |
| ILMN_2339410 | ACE | Inhibited | 0.774 | Downregulates (1) |
| ILMN_2340259 | PDE4B | Inhibited | 1.322 | Downregulates (1) |
| ILMN_2355225 | LSP1 | Activated | 0.766 | Upregulates (2) |
| ILMN_2358457 | ATF4 | Activated | 0.816 | Upregulates (1) |
| ILMN_2374778 | DUT | Activated | −1.556 | Downregulates (7) |
| ILMN_2379080 | NFATC2IP | Affected | −0.713 | Regulates (1) |
| ILMN_2379788 | HIF1A | Inhibited | 1.157 | Downregulates (9) |
| ILMN_2383349 | STEAP3 | Inhibited | −1.089 | Upregulates (3) |
| ILMN_2388155 | CASP3 | Activated | −0.889 | Downregulates (2) |
| ILMN_2392274 | CD82 | Activated | 0.623 | Upregulates (2) |
| ILMN_2406815 | LRRC17 | Activated | 0.993 | Upregulates (2) |
| ILMN_2408543 | PLAUR | Inhibited | 1.064 | Downregulates (4) |
| ILMN_2414399 | NME1 | Activated | 0.748 | Upregulates (1) |
| ILMN_3243142 | KAT2B | Affected | −0.837 | Regulates (2) |
| ILMN_3251232 | HMGN2 | Inhibited | −1.286 | Upregulates (2) |
| ILMN_3251283 | HDAC2 | Inhibited | −0.889 | Upregulates (1) |
| ILMN_3251550 | PHLDA1 | Activated | 0.895 | Upregulates (1) |
| ILMN_3305938 | SGK1 | Activated | 1.084 | Upregulates (6) |

In further aspects, a cancer may be identified as an immune signature or immune infiltrating bladder cancer (e.g., an immune infiltrating bladder cancer). In some aspects, a cancer is determined to be an immune infiltrating bladder cancer by assaying the expression of genes (e.g., two or more genes) in the cancer. For example, an immune infiltrating bladder cancer can be a cancer determined to have elevated expression (as compared to a reference level) of one, two, three or more of the genes as indicated in Table E.

TABLE E
Genes with elevated in immune infiltrating bladder cancer.

AIF1
BCL2
BTLA
CCL5
CD200R1
CD33
CD40
CD8B
CSF1
CTLA4
FASLG
FYB
FYN
HIVEP3
HLA-DRB6
ICAM3
IL10
IL12RB1
IL21R
IL4I1
TNFSF14
TRAF1
TRAFD1
VAV1
ZAP70

Additional biomarkers for use according to the embodiments are provided for instance in International Patent Application No. PCT/US2011/026329 and U.S. Publn. 2013/0084241 (each of which is incorporated herein by reference).

Creation of an intelligent system based on artificial intelligence, capable to predict clinical outcome with accuracy reaching 100% and taking as input a panel of molecular factors chosen through biological knowledge. Classification and Regression Trees (CART; see, e.g., Breiman et al. 1984, incorporated herein by reference) decision trees (DT; see e.g., Koza 1992, incorporated herein by reference) and Genetic Programming (GP) are the methods the inventors used to analyze the data. An original implementation of a DT and a GP system resulted into a model/equation using only a few molecular markers that created a model with 100% predictive accuracy for bladder cancer progression. This methodology can be adapted to various clinical questions that relate to outcomes after standard therapy or predict the best therapeutic combination for the best clinical outcome. Multiple systems which correspond to specific clinical questions may be implemented. Based on an original program, it can expand to include imaging data as a more objective quantification of relapse/progression criteria or as a measure of tissue modification (3D measurement and optical density variations).

### IV. Expression Assessment

In certain aspects, this invention entails measuring expression of one or more prognostic biomarkers in a sample of cells from a subject with cancer. The expression information may be obtained by testing cancer samples by a lab, a technician, a device, or a clinician. In a certain embodiment, the differential expression of one or more biomarkers including those of Tables A-E may be measured.

The pattern or signature of expression in each cancer sample may then be used to generate a cancer prognosis or classification, such as predicting cancer survival or recurrence. The level of expression of a biomarker may be increased or decreased in a subject relative to a reference level. The expression of a biomarker may be higher in long-term survivors than in short-term survivors. Alternatively, the expression of a biomarker may be higher in short-term survivors than in long-term survivors.

Expression of one or more of biomarkers identified by the inventors could be assessed to predict or report prognosis or prescribe treatment options for cancer patients, especially bladder cancer patients.

The expression of one or more biomarkers may be measured by a variety of techniques that are well known in the art. Quantifying the levels of the messenger RNA (mRNA) of a biomarker may be used to measure the expression of the biomarker. Alternatively, quantifying the levels of the protein product of a biomarker may be to measure the expression of the biomarker. Additional information regarding the methods discussed below may be found in Ausubel et al., (2003) Current Protocols in Molecular Biology, John Wiley &amp; Sons, New York, N.Y., or Sambrook et al. (1989) Molecular Cloning: A Laboratory Manual, Cold Spring Harbor Press, Cold Spring Harbor, N.Y. One skilled in the art will know which parameters may be manipulated to optimize detection of the mRNA or protein of interest.

A nucleic acid microarray may be used to quantify the differential expression of a plurality of biomarkers. Microarray analysis may be performed using commercially available equipment, following manufacturer's protocols, such as by using the Affymetrix GeneChip® technology (Santa Clara, Calif.) or the Microarray System from lncyte (Fremont, Calif.). Typically, single-stranded nucleic acids (e.g., cDNAs or oligonucleotides) are plated, or arrayed, on a microchip substrate. The arrayed sequences are then hybridized with specific nucleic acid probes from the cells of interest. Fluorescently labeled cDNA probes may be generated through incorporation of fluorescently labeled deoxynucleotides by reverse transcription of RNA extracted from the cells of interest. Alternatively, the RNA may be amplified by in vitro transcription and labeled with a marker, such as biotin. The labeled probes are then hybridized to the immobilized nucleic acids on the microchip under highly stringent conditions. After stringent washing to remove the non-specifically bound probes, the chip is scanned by confocal laser microscopy or by another detection method, such as a CCD camera. The raw fluorescence intensity data in the hybridization files are generally preprocessed with the robust multichip average (RMA) algorithm to generate expression values.

Quantitative real-time PCR (qRT-PCR) may also be used to measure the differential expression of a plurality of biomarkers. In qRT-PCR, the RNA template is generally reverse transcribed into cDNA, which is then amplified via a PCR reaction. The amount of PCR product is followed cycle-by-cycle in real time, which allows for determination of the initial concentrations of mRNA. To measure the amount of PCR product, the reaction may be performed in the presence of a fluorescent dye, such as SYBR Green, which binds to double-stranded DNA. The reaction may also be performed with a fluorescent reporter probe that is specific for the DNA being amplified.

A non-limiting example of a fluorescent reporter probe is a TaqMan® probe (Applied Biosystems, Foster City, Calif.). The fluorescent reporter probe fluoresces when the quencher is removed during the PCR extension cycle. Multiplex qRT-PCR may be performed by using multiple gene-specific reporter probes, each of which contains a different fluorophore. Fluorescence values are recorded during each cycle and represent the amount of product amplified to that point in the amplification reaction. To minimize errors and reduce any sample-to-sample variation, qRT-PCR is typically performed using a reference standard. The ideal reference standard is expressed at a constant level among different tissues, and is unaffected by the experimental treatment.

Suitable reference standards include, but are not limited to, mRNAs for the housekeeping genes glyceraldehyde-3-phosphate-dehydrogenase (GAPDH) and β-actin. The level of mRNA in the original sample or the fold change in expression of each biomarker may be determined using calculations well known in the art.

Immunohistochemical staining may also be used to measure the differential expression of a plurality of biomarkers. This method enables the localization of a protein in the cells of a tissue section by interaction of the protein with a specific antibody. For this, the tissue may be fixed in formaldehyde or another suitable fixative, embedded in wax or plastic, and cut into thin sections (from about 0.1 mm to several mm thick) using a microtome. Alternatively, the tissue may be frozen and cut into thin sections using a cryostat. The sections of tissue may be arrayed onto and affixed to a solid surface (i.e., a tissue microarray). The sections of tissue are incubated with a primary antibody against the antigen of interest, followed by washes to remove the unbound antibodies. The primary antibody may be coupled to a detection system, or the primary antibody may be detected with a secondary antibody that is coupled to a detection system. The detection system may be a fluorophore or it may be an enzyme, such as horseradish peroxidase or alkaline phosphatase, which can convert a substrate into a colorimetric, fluorescent, or chemiluminescent product. The stained tissue sections are generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for the biomarker.

An enzyme-linked immunosorbent assay, or ELISA, may be used to measure the differential expression of a plurality of biomarkers. There are many variations of an ELISA assay. All are based on the immobilization of an antigen or antibody on a solid surface, generally a microtiter plate. The original ELISA method comprises preparing a sample containing the biomarker proteins of interest, coating the wells of a microtiter plate with the sample, incubating each well with a primary antibody that recognizes a specific antigen, washing away the unbound antibody, and then detecting the antibody-antigen complexes. The antibody-antibody complexes may be detected directly. For this, the primary antibodies are conjugated to a detection system, such as an enzyme that produces a detectable product. The antibody-antibody complexes may be detected indirectly. For this, the primary antibody is detected by a secondary antibody that is conjugated to a detection system, as described above. The microtiter plate is then scanned and the raw intensity data may be converted into expression values using means known in the art.

An antibody microarray may also be used to measure the differential expression of a plurality of biomarkers. For this, a plurality of antibodies is arrayed and covalently attached to the surface of the microarray or biochip. A protein extract containing the biomarker proteins of interest is generally labeled with a fluorescent dye.

The labeled biomarker proteins may be incubated with the antibody microarray. After washes to remove the unbound proteins, the microarray is scanned. The raw fluorescent intensity data maybe converted into expression values using means known in the art.

Luminex multiplexing microspheres may also be used to measure the differential expression of a plurality of biomarkers. These microscopic polystyrene beads are internally color-coded with fluorescent dyes, such that each bead has a unique spectral signature (of which there are up to 100). Beads with the same signature are tagged with a specific oligonucleotide or specific antibody that will bind the target of interest (i.e., biomarker mRNA or protein, respectively). The target, in turn, is also tagged with a fluorescent reporter. Hence, there are two sources of color, one from the bead and the other from the reporter molecule on the target. The beads are then incubated with the sample containing the targets, of which up 100 may be detected in one well. The small size/surface area of the beads and the three dimensional exposure of the beads to the targets allows for nearly solution-phase kinetics during the binding reaction. The captured targets are detected by high-tech fluidics based upon flow cytometry in which lasers excite the internal dyes that identify each bead and also any reporter dye captured during the assay. The data from the acquisition files may be converted into expression values using means known in the art.

In situ hybridization may also be used to measure the differential expression of a plurality of biomarkers. This method permits the localization of mRNAs of interest in the cells of a tissue section. For this method, the tissue may be frozen, or fixed and embedded, and then cut into thin sections, which are arrayed and affixed on a solid surface. The tissue sections are incubated with a labeled antisense probe that will hybridize with an mRNA of interest. The hybridization and washing steps are generally performed under highly stringent conditions. The probe may be labeled with a fluorophore or a small tag (such as biotin or digoxigenin) that may be detected by another protein or antibody, such that the labeled hybrid may be detected and visualized under a microscope. Multiple mRNAs may be detected simultaneously, provided each antisense probe has a distinguishable label. The hybridized tissue array is generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for each biomarker.

### V. Cancer Treatments

In certain aspects, there may be provided methods for treating a subject determined to have cancer and with a predetermined expression profile of one or more biomarkers disclosed herein.

In a further aspect, biomarkers and related systems that can establish a prognosis of cancer patients in this invention can be used to identify patients who may get benefit of conventional single or combined modality therapy. In the same way, the invention can identify those patients who do not get much benefit from such conventional single or combined modality therapy and can offer them alternative treatment(s). For example, biomarker analyze may indicat whether the patient should be treated with a chemotherapeutic (such as an anti-mitotic therapy (e.g., cisplatin), an FGFR inhibitor, a BCG therapy, a surgical therapy or a radiation therapy.

In certain aspects of the present invention, conventional cancer therapy may be applied to a subject wherein the subject is identified or reported as having a good prognosis based on the assessment of the biomarkers as disclosed. On the other hand, at least an alternative cancer therapy may be prescribed, as used alone or in combination with conventional cancer therapy, if a poor prognosis is determined by the disclosed methods, systems, or kits.

Conventional cancer therapies include one or more selected from the group of chemical or radiation based treatments and surgery. Chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

Radiation therapy that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Laser therapy is the use of high-intensity light to destroy tumor cells. Laser therapy affects the cells only in the treated area. Laser therapy may be used to destroy cancerous tissue and relieve a blockage in the esophagus when the cancer cannot be removed by surgery. The relief of a blockage can help to reduce symptoms, especially swallowing problems.

Photodynamic therapy (PDT), a type of laser therapy, involves the use of drugs that are absorbed by cancer cells; when exposed to a special light, the drugs become active and destroy the cancer cells. PDT may be used to relieve symptoms of esophageal cancer such as difficulty swallowing.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

Alternative cancer therapy include any cancer therapy other than surgery, chemotherapy and radiation therapy in the present invention, such as immunotherapy, gene therapy, hormonal therapy or a combination thereof. Subjects identified with poor prognosis using the present methods may not have favorable response to conventional treatment(s) alone and may be prescribed or administered one or more alternative cancer therapy per se or in combination with one or more conventional treatments.

For example, the alternative cancer therapy may be a targeted therapy. The targeted therapy may be an anti-FGFR treatment. In one embodiment of the method of the invention, the anti-FGFR agent used is a tyrosine kinase inhibitor.

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Gene therapy is the insertion of polynucleotides, including DNA or RNA, into an individual's cells and tissues to treat a disease. Antisense therapy is also a form of gene therapy in the present invention. A therapeutic polynucleotide may be administered before, after, or at the same time of a first cancer therapy. Delivery of a vector encoding a variety of proteins is encompassed within the invention. For example, cellular expression of the exogenous tumor suppressor oncogenes would exert their function to inhibit excessive cellular proliferation, such as p53, p16 and C-CAM.

Additional agents to be used to improve the therapeutic efficacy of treatment include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers Immunomodulatory agents include tumor necrosis factor; interferon alpha, beta, and gamma; IL-2 and other cytokines; F42K and other cytokine analogs; or MIP-1, MIP-lbeta, MCP-1, RANTES, and other chemokines. It is further contemplated that the upregulation of cell surface receptors or their ligands such as Fas/Fas ligand, DR4 or DRS/TRAIL would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### VI. Kits

Certain aspects of the present invention also encompass kits for performing the diagnostic and prognostic methods of the invention. Such kits can be prepared from readily available materials and reagents. For example, such kits can comprise any one or more of the following materials: enzymes, reaction tubes, buffers, detergent, primers, probes, antibodies. In a preferred embodiment, these kits allow a practitioner to obtain samples of neoplastic cells in blood, tears, semen, saliva, urine, tissue, serum, stool, sputum, cerebrospinal fluid and supernatant from cell lysate. In another preferred embodiment these kits include the needed apparatus for performing RNA extraction, RT-PCR, and gel electrophoresis. Instructions for performing the assays can also be included in the kits.

In a particular aspect, these kits may comprise a plurality of agents for assessing the differential expression of a plurality of biomarkers, for example, two, three, four or more of the genes of Tables A-E wherein the kit is housed in a container. The kits may further comprise instructions for using the kit for assessing expression, means for converting the expression data into expression values and/or means for analyzing the expression values to generate prognosis. The agents in the kit for measuring biomarker expression may comprise a plurality of PCR probes and/or primers for qRT-PCR and/or a plurality of antibody or fragments thereof for assessing expression of the biomarkers. In another embodiment, the agents in the kit for measuring biomarker expression may comprise an array of polynucleotides complementary to the mRNAs of the biomarkers of the invention. Possible means for converting the expression data into expression values and for analyzing the expression values to generate scores that predict survival or prognosis may be also included.

Kits may comprise a container with a label. Suitable containers include, for example, bottles, vials, and test tubes. The containers may be formed from a variety of materials such as glass or plastic. The container may hold a composition which includes a probe that is useful for prognostic or non-prognostic applications, such as described above. The label on the container may indicate that the composition is used for a specific prognostic or non-prognostic application, and may also indicate directions for either in vivo or in vitro use, such as those described above. The kit of the invention will typically comprise the container described above and one or more other containers comprising materials desirable from a commercial and user standpoint, including buffers, diluents, filters, needles, syringes, and package inserts with instructions for use.

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Human Specimens:

Tumors and clinical data were obtained from the MD Anderson genitourinary cancers research database. Fresh frozen tissues were obtained from the SPORE Tissue Core. Of note, all patients had previously signed informed consent allowing collection of their tissue and of their clinical data in the genitourinary research database. An additional institutional review board (IRB) approved protocol was obtained for the specific analyses described herein and all tissue samples had a review from a pathologist. Patients were classified as muscle invasive for tumor growth into the muscularis propria; otherwise, they were classified as non-muscle invasive. Total RNA from human specimens were isolated using mirVana miRNA isolation kit (Ambion, Inc).

Cell Lines:

All cell lines were obtained from the MD Anderson Bladder SPORE Tissue Bank, and their identities were validated by DNA fingerprinting using AmpFISTR® Identifiler® Amplification kit (Applied Biosystems, Foster City, Calif.), performed by the MD Anderson Characterized Cell Line Core. Cell lines were cultured in modified Eagle's MEM supplemented with 10% fetal bovine serum, vitamins, sodium pyruvate, L-glutamine, penicillin, streptomycin, and nonessential amino acids at 37° C. in 5% CO₂ incubator. To generate p63 stable knock down cells, the pan p63 targeting lentiviral shRNA construct (Open Biosystems, V3LHS_{—}397885) and pGIPZ lentiviral empty vector (Open Biosystems, RHS4339) were transfected into 293T cells in order to generate lentivirus. UC14 cells were plated on a 6-well plate (12×10⁴ cells/well), and medium containing lentiviral particles was added after 24 h. Cells were incubated with lentivirus for 16 h, and were then washed and cultured in fresh medium. Fluorescence-activated cell sorting (FACS) was performed after 4-5 d to isolate GFP positive cells, and these cells were then cultured in medium containing puromycin (4 mg/ml).

Microarray Experiments and Data Processing:

RNA purity and integrity were measured by NanoDrop ND-1000 and Agilent Bioanalyzer and only high quality RNA was used for the cRNA amplification. The synthesis of biotin labeled cRNA which was prepared using the Illumina RNA amplification kit (Ambion, Inc, Austin, Tex.) and amplified cRNA was hybridized to Illumina HT12 V3 chips (Illumina, Inc., San Diego, Calif.). After being washed, the slides were scanned with Bead Station 500× (Illumina, Inc.) and the signal intensities were quantified with GenomeStudio (Illumina, Inc.). Quantile normalization was used to normalize the data. BRB ArrayTools version 4.2 developed by National Cancer Institute [7] was used to analyze the data. To select genes that were differentially expressed between the three different sub-groups (Cluster 1, 2, 3), a class comparison tool within BRB ArrayTools was used. This software uses a two-sample t-test to calculate the significance of the observations with false discovery rate (FDR) (i.e., P<0.001). To see expression patterns of genes, specific gene expression values, adjusted to a mean of zero, were used for clustering with Cluster and TreeView (Eisen et al., 1998).

Pathway Analysis:

Functional and pathway analyses were performed using Ingenuity Pathway Analysis (IPA) software (Ingenuity® Systems, CA). This software contains a database for identifying networks and pathways of interest in genomic data. The “molecular and cellular function” and “upstream regulator” categories within IPA were used to interpret the biological properties of the subsets in the bladder tumors.

Real-time Reverse Transcriptase PCR Analysis:

p63, STAT3, and PPARγ target genes were analyzed by real-time PCR. Real-time PCR technology (StepOne; Applied Biosystems, Foster City, Calif.) was used in conjunction with Assays-on-Demand (Applied Biosystems). The comparative CT method (Livak et al., 2001) was used to determine relative gene expression levels for each target gene, cyclophilin A gene was used as an internal control to normalize for the amount of amplifiable RNA in each reaction. Taqman primers for p63, cytokeratin 5 (KRT5), cytokeratin 14 (KRT14), Cyclophylin A were purchased from Applied Biosystems.

### Example 2

### Results

Initial Gene Expression Profiling Reveals 3 Subsets of Muscle Invasive Bladder Cancers.

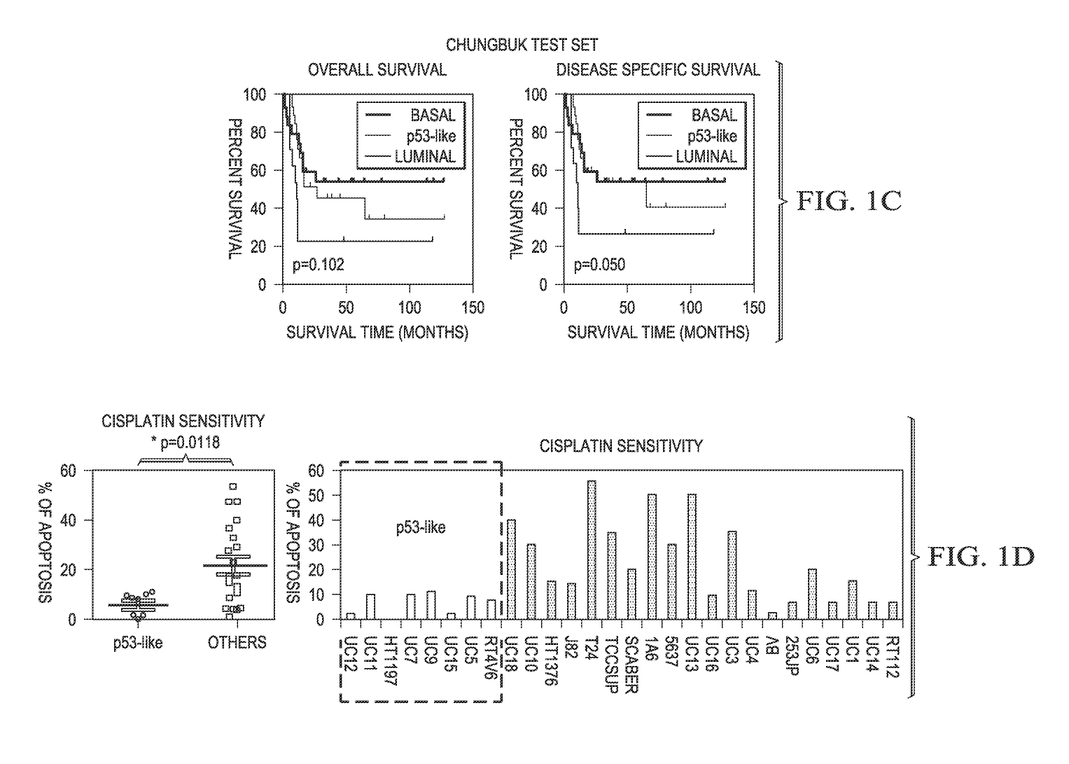

The inventors performed whole genome mRNA expression analyses on 73 primary muscle-invasive urothelial tumors (Table 4A). The majority of the patients had a mean age of 68.8 years (SD+/−10.2), were Caucasian (54, 74%), and of male gender male (54, 74%). Table 4A depicts the clinicopathologic characteristics of this cohort by subtype designation. Tumors were characterized using TNM staging (reference) at time of diagnosis by transurethral resection (clinical stage) and after cystectomy (pathologic stage). There were no significant differences between groups based on age, race, gender, clinical T stage, or the use of neoadjuvant chemotherapy. The inventors performed unsupervised cluster analysis using the 6700 probes that exhibited expression ratios of at least 2-fold relative to the median gene expression level across all tissues in at least 7 tissues. The tumors formed three distinct clusters, and the top 10 genes that determined membership within each cluster are: Cluster 1 (KRT14, DSG3, KRT6B, KRT5, KRT6A, KRT6C, LOC653499, LOC728910, PI3 and S100A7); Cluster 2 (ACTG2, CNN1, MYH11, MFAP4, PGM5, FLNC, ACTC1, DES, PCP4 and DMN); and Cluster 3 (MAL, FMO9P, BHMT, SNX31, KRT20, SPINK1, DHRS2, UPK2, UPK1A and VSIG2). Kaplan-Meier survival outcomes analyses of each cluster revealed that cluster 1 was associated with the poorest clinical outcomes (p<0.05), whereas those associated with clusters 2 and 3 were not significantly different from each other (FIG. 1). Patients in cluster 1 had more tumors with squamous or sarcomatoid components and advanced disease at presentation (Table 4A). Most importantly the basal subtype had a distinctly poor disease specific survival (median 14.9 months) compared to the other subtypes (FIG. 1, p=0.028). There was a trend toward poor overall survival in this group, however statistical significance was not reached (FIG. 1, p=0.098). Patients in cluster 2 were more likely to have clinically localized disease at time of presentation and a higher rate of cystectomy but also the highest proportion of node positive disease after surgery (Table 4A). Patients in cluster 3 were also characterized by organ-confined disease after cystectomy with standard urothelial histology (Table 4A).

Activating mutations in Ras and FGFR3 and inactivating mutations in TP53 and Rb are frequently observed in muscle invasive bladder cancers (Cote et al., 1998). The inventors therefore performed exome sequencing on all tissues that were available to determine the frequencies of these mutations within the 3 bladder cancer clusters. Cluster 3 contained the largest fraction of tumors with activating FGFR3 mutations, while mutations in Rb appeared to be more prevalent in cluster 1, and p53 mutation levels were equivalent in clusters 1 and 3. The inventors detected only three activating Ras mutations within the entire cohort.

The inventors then performed molecular pathway analyses to identify candidate biological mechanisms leading to the emergence of the 3 bladder cancer subsets. The inventors extracted the significantly differentially expressed genes in each subset using the class comparison tool of BRB Array Tools (p<0.001 with FDR <0.1). The inventors then subjected the genes to IPA analyses and identified the biological characteristics that characterized each cluster. Tables 1A-C show the top 3 significant “molecular and cellular functions” for clusters 1, 2, and 3, respectively. The inventors predicted the activation status of each function based on bias-corrected z-scores (−2>Z or 2<Z), and if the inventors observed more than 3 functional annotations within each category, then the inventors presented the top 3 functional annotations in Table 1. “Cellular movement” and “cellular growth and proliferation” were significantly enriched in all three subsets, but both were “activated” only in the lethal subset 1, whereas both were “decreased” in subsets 2 and 3. Therefore, cluster 1 appeared to be enriched for tumors that were more migratory and proliferative, consistent with the observation that they were associated with poor clinical outcomes.

In order to determine the robustness of the 3 subsets, the inventors performed GEP on a separate cohort of 56 muscle-invasive tumors and used the gene sets defined in the discovery cohort to determine membership within each cluster. To determine whether their approach could be used on routinely collected formalin-fixed, paraffin-embedded (FFPE) tissue sections, the inventors used marked H&E-stained adjacent sections to manually macrodissect tumor areas from 5-10 consecutive 10 μm unstained sections, isolated total RNA, and performed whole genome GEP using the Illumina DASL platform. Consistent with the results obtained in the discovery cohort, the tumors formed 3 distinct clusters, and disease-specific survival was significantly worse in patients whose tumors were contained within cluster 1. To provide further validation of the approach, the inventors also performed cluster analyses on muscle-invasive tumors from two additional, publically available bladder cancer GEP datasets (Korean and Swedish). The Korean cohort was previously described by Kim et al. and originally included 165 fresh frozen tumors from both transurethral resection and cystectomy specimens (Table 4B). The Korean tumors formed 3 distinct clusters). Based on the available clinical data and restricting the analyses to muscle invasive tumors only (n=55), the three subtypes did not differ by clinical stage or treatment with systemic therapy (given for clinically metastatic, or pT3+/N+ disease after cystectomy). No information on tumor histology or pathologic outcome was available (Table 4B). There was a difference in age (patients in cluster 3 were older) and gender (higher proportion of females in the basal cluster). Median DSS (11.2 months, p=0.102) and OS (10.4 months, p=0.058) were lowest in the basal cluster as observed in the training dataset; however this did not reach statistical significance when compared with the other subtypes. The Swedish cohort was comprised of 308 tumors collected by transurethral biopsy, of which 93 were muscle invasive tumors. Again, the 3 major clusters the inventors observed in their discovery set were readily detected in the Swedish cohort, and the cluster 1 tumors were associated with shorter disease-specific and overall survival. Overall, the results indicate that the cluster 1 muscle-invasive tumors display a reproducibly lethal phenotype across 4 independent datasets. Furthermore, the data show that the 3 subsets can be easily identified using DASL on routinely collected FFPE tissue sections.

Identification of Upstream Regulators.

To elucidate the molecular drivers of gene expression within each cluster, the inventors used the “upstream regulators” function in IPA to identify transcription factors that might drive the gene expression patterns associated with clusters 1, 2 and 3. Table 2 displays the top 10 “activated” and “inhibited” upstream regulators within each subset. The STAT3, NFκB, and p63 transcriptional pathways were all significantly “activated” in cluster 1 tumors (Table 2). STAT3 and NFκB have been widely implicated in cancer progression, and the involvement of p63 in driving cluster 1 gene expression was consistent with the inventor's previous work that established that ΔNp63α expression is associated with the poor clinical outcomes (Choi et al., 2012; Karni-Schmidt et al., 2011). Further inspection of the cluster 1 signature confirmed that it contains genes that are likely to be direct transcriptional targets of ΔNp63. Specifically, 6 of the 10 top upregulated molecules (KRT6A, PI3, KRT14, KRT6C, KRT5 and S100A7) based on log ratio in cluster 1 have been reported to be direct p63 targets, and the inventors confirmed that they contain consensus p53 response elements within their promoters. Cluster 1 was also enriched for Myc expression. Using quantitative RT-PCR, the inventors also confirmed their previous observation that p63 mRNA expression was significantly elevated in the lethal cluster 1 compared to clusters 2 and 3. Importantly, several of the p63-associated markers that characterized cluster 1 (CD44, KRT5, KRT6, KRT14, CDH3) are also well-established markers of the basal breast cancer subset, and p63, KRT4, and KRT14 are markers of the basal layer of the normal urothelium. Therefore, cluster 1 contained muscle-invasive bladder cancers that possessed a p63-associated basal molecular phenotype. Further analysis showed that Snail (SNAI2), Zeb2, and vimentin (VIM) are overexpressed in cluster 1 (p<0.001 with FDR<0.001) indicating that the basal subset is mesenchymal.

Tumors within cluster 2 were characterized by gene expression patterns associated with active tumor suppressors (p53, CDKN2A (p16) and RB) and suppressed E2F pathway genes (Table 2). The relative expression of p53 and CDKN2A pathway genes, which included regulators of the mitotic cell cycle (AURKA, AURKB, MAD2L1) and S phase (CCNE1, CCNA2, CHEK1) was also observed. The prevalence of p53 mutations appeared to be lower in cluster 2 tumors as compared to clusters 1 and 3. Therefore, cluster 2 appears to contain tumors with a “wild type p53-like” molecular phenotype.

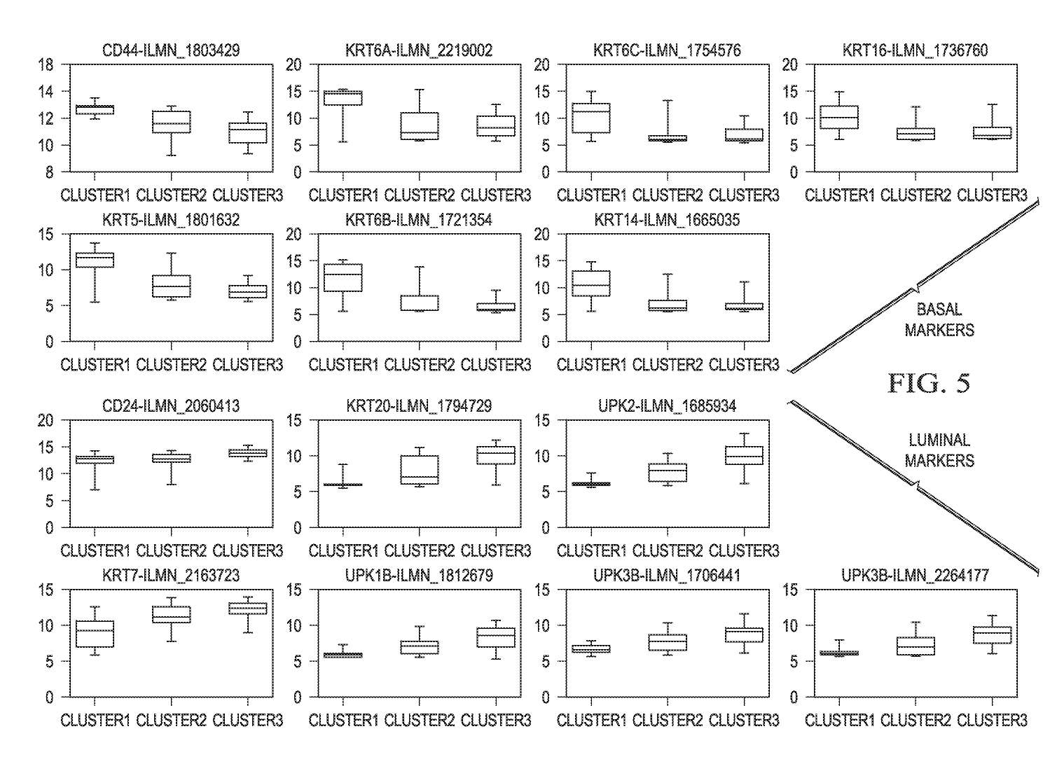

Interestingly, the estrogen receptor (ER) and its coactivator, TRIM24, were among the top “activated” upstream regulators in the tumors within cluster 3, whereas STAT3 and NFκB were among the top transcriptional pathways that were downregulated in these tumors (Table 2). Conversely, ER and TRIM24 were among the top downregulated pathways in the basal cluster 1 tumors (Table 2). Cluster 3 tumors also exhibited gene expression patterns consistent with activated peroxisome proliferator activator receptor (PPAR) signaling (Table 2); PPARγ is known to play a central role in urothelial luminal differentiation. Several of the genes that characterized cluster 3 tumors (CD24, FOXA1, ERBB2, ERBB3, GATA3, XBP1, and KRT20) are well-established markers for luminal breast cancers, and many of them contain canonical ER and/or PPAR response elements within their promoters. Gene set enrichment analyses confirmed that cluster 3 was enriched for luminal markers (FIG. 5, p=0.02), whereas cluster 1 was enriched for basal markers (FIG. 5, p=0.002). Interestingly, cluster 2 appeared to contain a mixture of tumors with non-overlapping basal or luminal features, with the majority of them displaying a more luminal phenotype.

Transcriptional Control of Basal and Luminal Gene Expression.

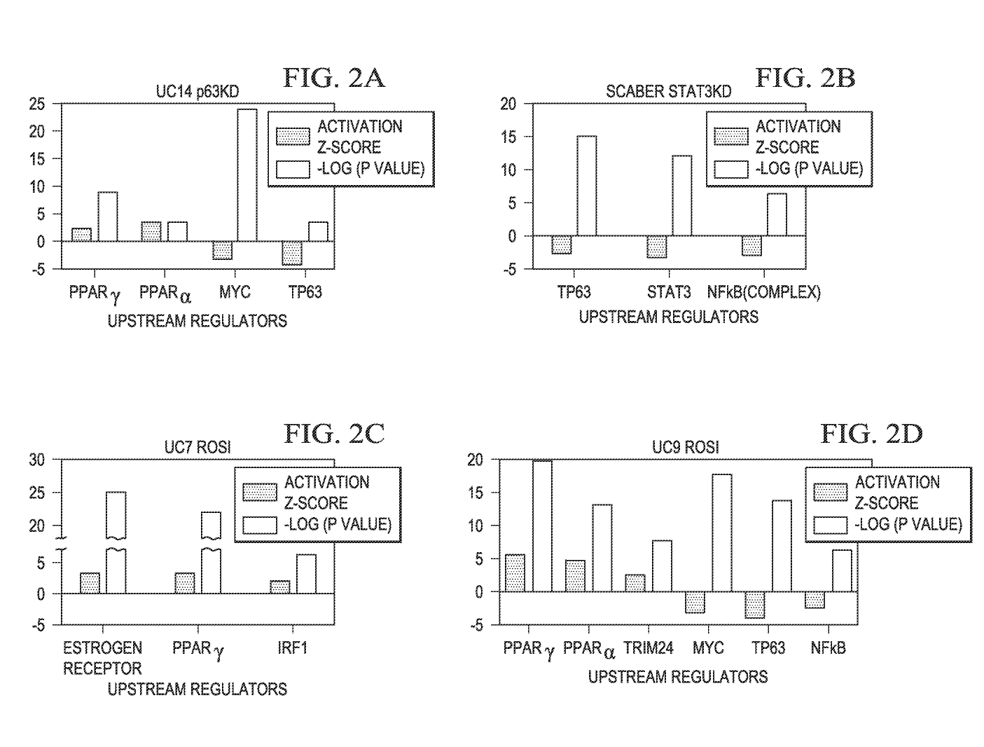

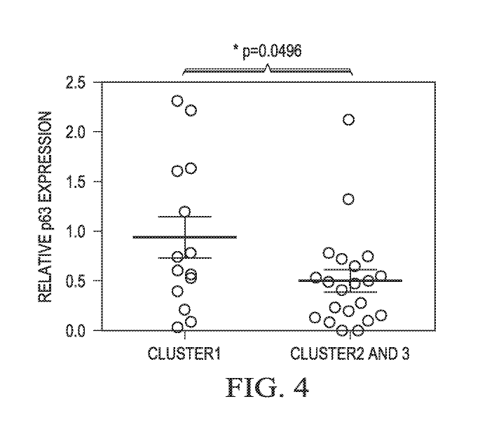

The inventors performed functional studies in human bladder cancer cell lines to more precisely define the molecular mechanisms that control the basal and luminal gene expression signatures. The inventors first examined patterns of basal and luminal gene expression across a set of 30 human bladder cancer cell lines. Many of the cell lines coexpressed basal and luminal markers, making it challenging to unequivocally assign them to the basal or luminal subset. Therefore, the inventors randomly selected one of the ΔNp63-positive lines (UM-UC14) to determine the effects of p63 silencing on basal gene expression. Pathway analyses of the gene expression profiles of UC14 cells transduced with non-targeting (NT) or p63-specific shRNA constructs revealed that PPAR signaling was increased and Myc signaling was decreased when p63 expression was suppressed (FIG. 2A). Furthermore, p63 knockdown resulted in downregulation of basal markers (CD44, CDH3, KRT5, KRT6) and upregulation of luminal markers (ERBB2, ERBB3, FOXA1, KRT8, KRT9, and UPKs) (FIG. 2A). To determine the effects of STAT3 on gene expression, the inventors used their cell line GEP dataset to identify the line with the highest basal STAT3 pathway gene expression (Scaber), transfected the cells with non-targeting or STAT3-specific siRNAs, and compared the whole genome expression profiles of the cells. Strikingly, the p63 and NFκB pathways were both significantly downregulated in parallel with STAT3 (FIG. 2B), strongly suggesting that both pathways are downstream targets of STAT3 signaling in the cells. To define the role of PPAR signaling in controlling basal and luminal gene expression, the inventors examined the effects of the PPARγ-selective agonist rosiglitazone on gene expression in two of their cell lines that displayed low p63 expression (UC7 and UC9). In both cell lines PPAR pathway gene expression was strongly induced (FIGS. 2C,D). In addition, in the UC7 cells rosiglitazone induced expression of the ER-, PPARγ-, and IRF-1-transcriptional pathways (FIG. 2C), whereas in UC9 it increased TRIM24 pathway activity and downregulated the Myc, p63, and NFκB pathways (FIG. 4D). IRF-1 has been implicated in urothelial differentiation downstream of PPARγ in normal urothelial cells. Rosiglitazone decreased expression of basal markers and increased expression of luminal markers in both cell lines (FIGS. 2C,D). Together, these results confirm that STAT3 and p63 directly control basal gene expression, whereas PPARγ directly controls luminal gene expression. The results of these functional studies and the upstream pathway analyses of primary tumors (Table 2) also demonstrate that the basal and luminal transcription factors antagonize each other.

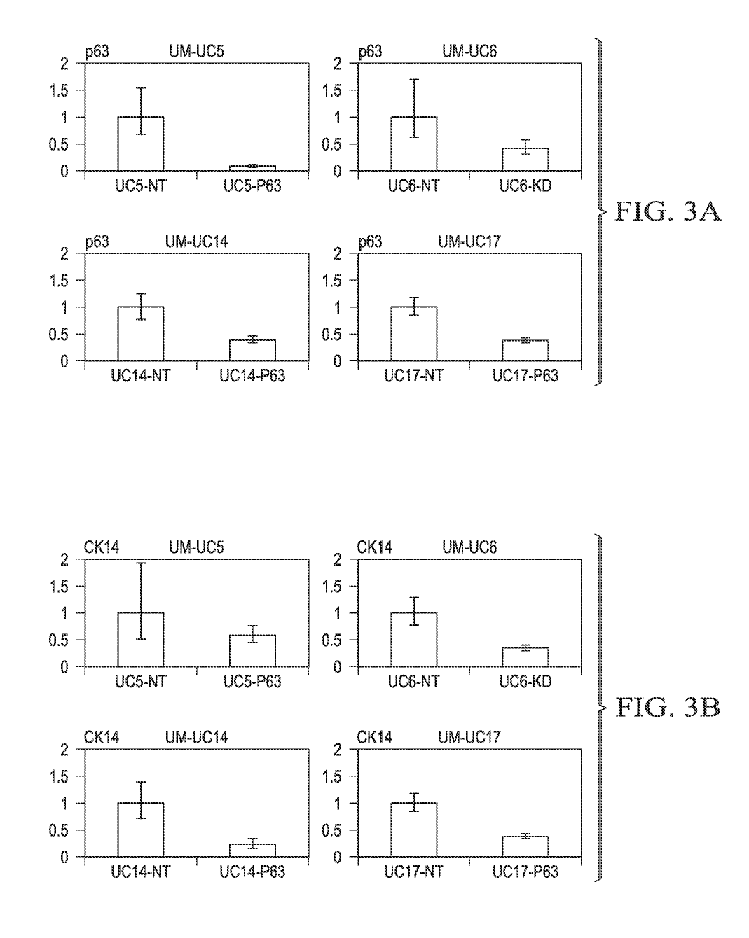

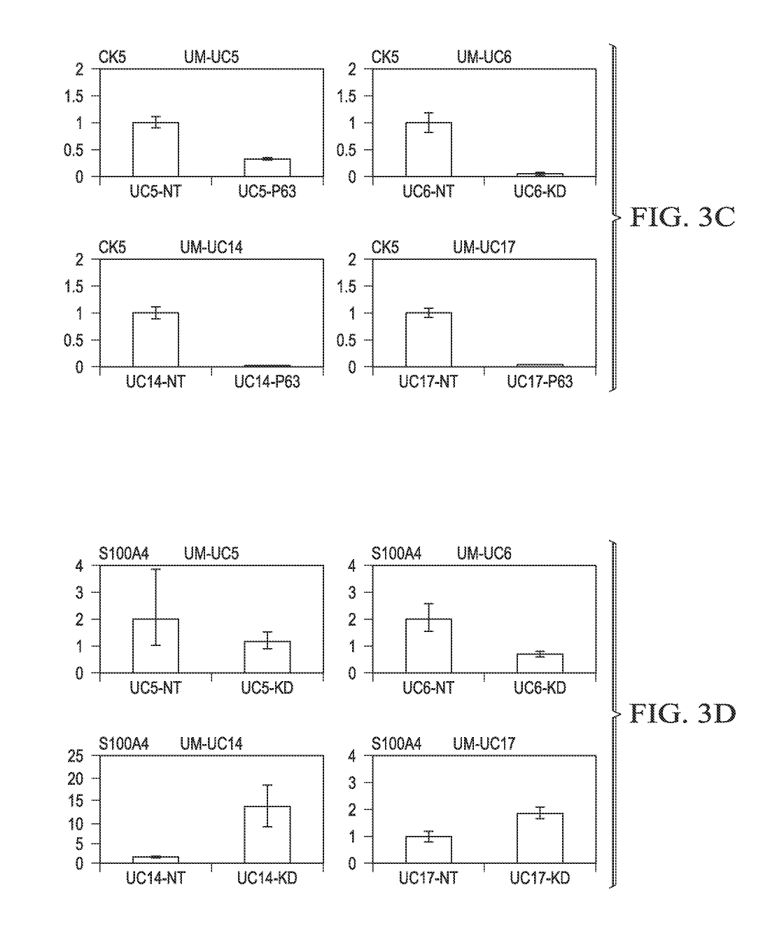

To further confirm p63's role in controlling basal gene expression, the inventors stably knocked down p63 expression in 3 additional human bladder cancer cell lines and used quantitative RT-PCR to measure basal marker expression. One (UM-UC5) was generated from a squamous tumor and is therefore likely to have a basal origin, whereas the other 3 (UM-UC6, UM-UC14, and UM-UC17) contain activating FGFR3 mutations and constitutively express PPARγ pathway genes, so the inventors suspect that they were luminal in origin but acquired mixed basal/luminal features in tissue culture. Stable p63 knockdown (FIG. 3A) decreased expression of the basal markers KRT14 (FIG. 3B) and KRT5 (FIG. 3C) and increased expression of S100A4 (FIG. 3D) in all 4 cell lines, confirming that their expression was controlled by p63.

Because STAT3 is known to be activated by the epidermal growth factor receptor (EGFR) in epithelial tumors and basal tumors expressed relatively high levels of the EGFR and two of its ligands (HB-EGF and neuregulin), the inventors also examined the effects of the EGFR antagonist gefitinib (Iressa) on EGFR and STAT3 phosphorylation and p63 expression in the UC5 and Scaber cells. EGFR inhibition resulted in inhibition of STAT3 phosphorylation and downregulation of p63, P-cadherin, and cytokeratins 5 and 14, consistent with the idea that EGFR inhibition promotes luminal gene expression primarily by inhibiting the STAT3/p63 pathway.

Subset-Dependent Drug Sensitivity.

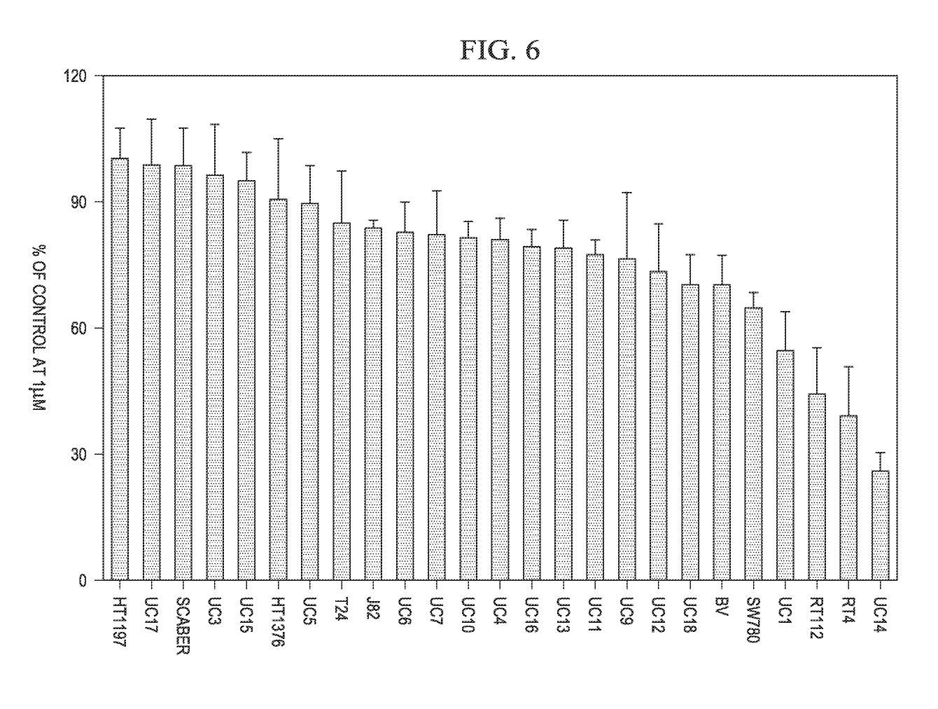

The prevalence of activating FGFR3 mutations within the luminal primary tumors and the cell lines that displayed luminal features suggested that the luminal subtype might be enriched for FGFR3 dependency. To test this possibility, the inventors examined the effects of the FGFR-selective inhibitor BGJ398 on proliferation in the inventor's panel of 30 human bladder cancer cell lines using MTT assays. All of the most drug-sensitive lines (SW780, UC1, RT112, RT4, and UC14) (FIG. 6) co-clustered together within the most luminal subset of bladder cancer cell lines, confirming that FGFR inhibitor sensitivity is confined to the cell lines that express a luminal gene expression signature.

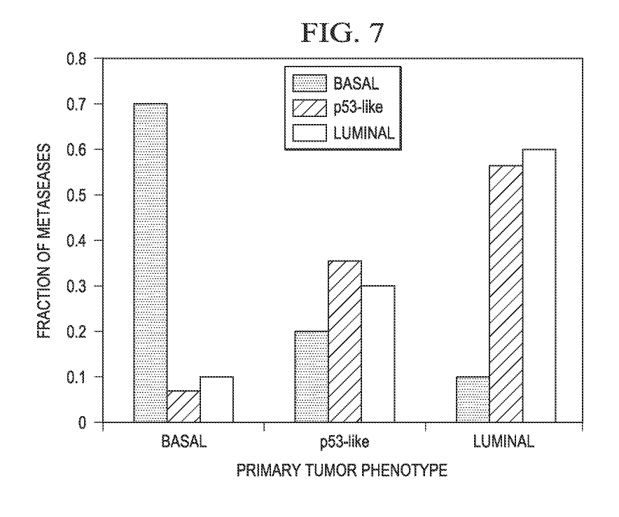

Presurgical (neoadjuvant) cisplatin-based chemotherapy is the current standard-of-care for high-risk muscle-invasive bladder cancer. Previous studies have demonstrated that complete pathological response (downstaging to pT1/pT0 at cystectomy) is a strong predictor of disease-specific survival in bladder cancer patients. To examine the relationship between the 3 molecular subsets and chemotherapy sensitivity, the inventors identified 18 patients within their discovery cohort that had received neoadjuvant chemotherapy and compared the pathological response rates within each cluster. Strikingly, whereas over half of the basal/cluster 1 (3/5) and luminal/cluster 3 (4/6) tumors responded to chemotherapy, none (0/7) of the p53-like cluster 2 tumors was downstaged in this initial cohort (Table 3). To test this relationship further, the inventors identified 16 additional tumors from a recently completed neoadjuvant MVAC/avastin clinical trial, 7 from patients whose tumors displayed complete pathological responses and 9 from patients whose tumors progressed on therapy. Gene expression profiling classified 3 of the tumors as basal, 4 as luminal, and 9 as p53-like (FIG. 7). All of the basal tumors (3/3) and half of the luminal tumors (2/4) responded to therapy, whereas most of the p53-like tumors (7/9) were resistant.

Molecular Subsets and Metastasis.

It is generally assumed that bladder cancer lethality correlates directly with metastatic potential. Because increased “cellular movement” was a characteristic feature of the basal gene expression signature and the basal tumors were associated with significantly worse clinical outcomes in patients, the inventors hypothesized that the basal primary tumors would produce metastases at higher frequencies than the other molecular subsets. To test this hypothesis, the inventors used DASL GEP to explore the relationship between subset membership and metastasis in a set of 33 matched primary tumors and lymph node metastases. The inventors also compared the primary tumors and metastases to determine whether any changes in subset membership had occurred. Of the 33 primary tumors, 9 were basal, 14 were p53-like, and 10 were luminal Therefore, primary tumors from all 3 clusters produced metastases. The majority of metastases from basal primary tumors remained basal (6/9). However, there was significant plasticity in the p53-like and luminal primary tumors, in that metastases from these tumors often switched to the other subset. This observation is consistent with the prevalence of luminal markers in the p53-like subset and raises the possibility that luminal tumors may easily become chemoresistant by switching to the p53-like molecular phenotype.

miR-200 and miR-205 Expression Identified Invasive Bladder Cancer with a Favorable Biology and Prognosis.

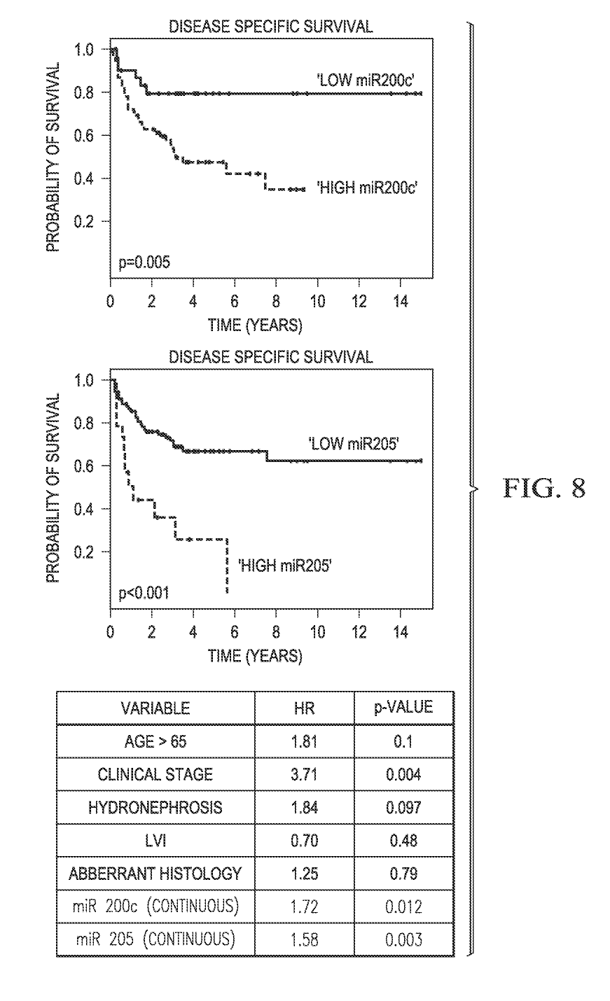

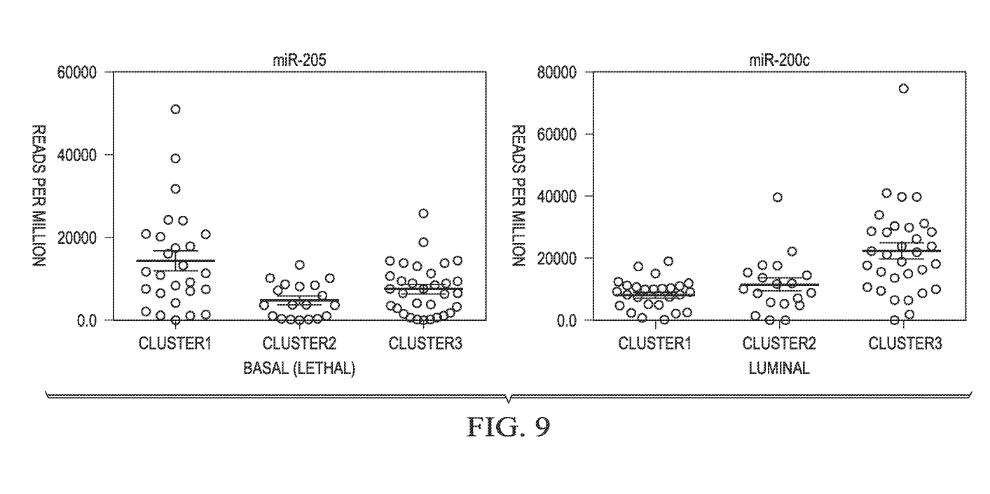

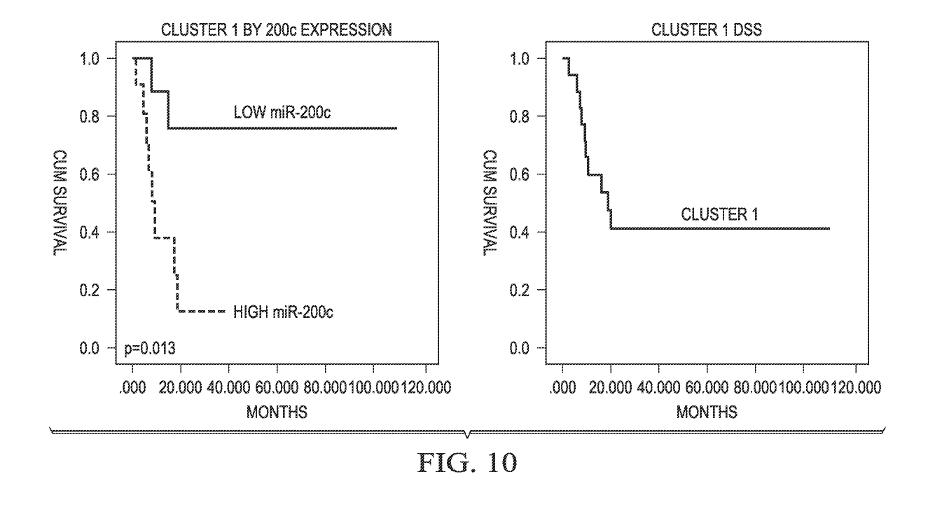

miR-200 and miR-205 prevent EMT by inhibiting Zeb1/2 and maintaining E-cadherin expression and an epithelial phenotype. The inventors measured expression of miR-200 and miR-205 and other EMT-related genes (Zeb-1/2) by GEP and RT-PCR on 101 tumors. Specimens were macro-dissected, and only those with greater than 80% tumor were analyzed. miR-200 and miR-205 expression were correlated with overall survival and disease specific survival (FIG. 8). The inventors then analyzed the expression of miR-200c and miR-205 in the above identified bladder cancer subsets. High miR-205 expression characterized the basal subset, while high miR-200c was expressed by the luminal clusters (FIG. 9). Furthermore, the inventors found that disease specific survival of the lethal subset correlates with miR-200c expression (FIG. 10). Therefore, the basal cluster is the lethal subset and is characterized by the expression of both mesenchymal (Snail, Zeb1/2, vimentin) and epithelail (miR-200c) genes.

TABLE 1A
Cluster 1

| | | | Predicted | | |
|---|---|---|---|---|---|
| Category | Functions Annotation | p value | Activation State | Bias-corrected Z score | # molecules |
| Cellular Movement | migration of cells | 4.21E−18 | Increased | 5.060 | 253 |
| | cell movement | 8.62E−17 | Increased | 5.368 | 271 |
| | cell movement of phagocytes | 1.04E−13 | Increased | 4.752 | 97 |
| Cellular Growth and | proliferation of cells | 1.09E−16 | Increased | 2.522 | 432 |
| Proliferation | proliferation of blood cells | 1.26E−08 | Increased | 2.339 | 120 |
| | proliferation of immune cells | 2.10E−07 | Increased | 2.290 | 110 |
| Cellular Function and | function of leukocytes | 8.26E−14 | Increased | 2.584 | 102 |
| Maintenance | function of blood cells | 1.26E−13 | Increased | 2.584 | 106 |
| | function of lymphocytes | 2.42E−08 | Increased | 2.158 | 60 |
| Antigen Presentation | chemotaxis of phagocytes | 1.07E−10 | Increased | 4.797 | 58 |
| | chemotaxis of neutrophils | 6.14E−09 | Increased | 4.464 | 36 |
| | immune response of phagocytes | 1.90E−05 | Increased | 2.038 | 31 |
| Cell-To-Cell Signaling | activation of cells | 1.49E−10 | Increased | 3.347 | 138 |
| and Interaction | activation of blood cells | 3.00E−10 | Increased | 3.241 | 112 |
| | activation of leukocytes | 7.45E−10 | Increased | 2.862 | 101 |

TABLE 1B
Cluster 2

| | | | Predicted | | |
|---|---|---|---|---|---|
| Category | Functions Annotation | p value | Activation State | Bias-corrected Z score | # molecules |
| Cell Cycle | M phase | 4.28E−06 | Decreased | −2.258 | 26 |
| | ploidy of cells | 4.81E−04 | Increased | 2.264 | 14 |
| | M phase of tumor cell lines | 4.87E−04 | Decreased | −2.203 | 11 |
| Cellular Assembly and | alignment of chromosomes | 8.04E−07 | Decreased | −2.133 | 9 |
| Organization | chromosomal congression of | 8.37E−07 | Decreased | −2.000 | 6 |
| | chromosomes | | | | |
| | organization of cytoplasm | 2.19E−05 | Increased | 2.856 | 100 |
| DNA Replication, | alignment of chromosomes | 8.04E−07 | Decreased | −2.133 | 9 |
| Recombination, and | chromosomal congression of | 8.37E−07 | Decreased | −2.000 | 6 |
| Repair | chromosomes | | | | |
| | checkpoint control | 8.11E−03 | Decreased | −2.177 | 10 |
| Cellular Growth and | proliferation of cells | 1.08E−06 | Decreased | −3.144 | 233 |
| Proliferation | proliferation of tumor cell lines | 9.62E−04 | Decreased | −3.327 | 91 |
| Cellular Movement | invasion of carcinoma cell lines | 1.90E−02 | Decreased | −2.773 | 8 |
| | cytokinesis | 3.76E−03 | Decreased | −2.578 | 14 |

TABLE 1C
Cluster 3

| | | | Predicted | Bias-corrected Z | |
|---|---|---|---|---|---|
| Category | Functions Annotation | p value | Activation State | score | # molecules |
| Cellular Movement | cell movement | 7.36E−23 | Decreased | −3.643 | 276 |
| | migration of cells | 2.32E−21 | Decreased | −3.660 | 250 |
| | cell movement of tumor cell lines | 1.88E−13 | Decreased | −3.186 | 107 |
| Cellular Development | differentiation of cells | 3.22E−13 | Decreased | −4.663 | 243 |
| | differentiation of connective tissue | | | | |
| | cells | 5.90E−05 | Decreased | −2.377 | 65 |
| | neuritogenesis | 1.29E−04 | Decreased | −2.964 | 58 |
| Cellular Growth and | proliferation of pericytes | 5.73E−04 | Decreased | −2.967 | 10 |
| Proliferation | proliferation of hepatic stellate cells | 1.31E−03 | Decreased | −2.795 | 9 |
| Cellular Assembly and | fibrogenesis | 5.99E−11 | Decreased | −2.811 | 67 |
| Organization | formation of filaments | 4.64E−10 | Decreased | −2.615 | 64 |
| | organization of cytoskeleton | 4.08E−08 | Decreased | −3.812 | 149 |
| Cell-To-Cell Signaling | binding of cells | 2.49E−10 | Decreased | −2.238 | 83 |
| and Interaction | adhesion of tumor cell lines | 4.62E−08 | Decreased | −3.362 | 44 |
| | adhesion of immune cells | 1.02E−06 | Decreased | −4.576 | 53 |

TABLE 2
Upstream regulators in each cluster

| | Predicted Activation State: Activated | | | Predicted Activation State: Inhibited | | |
|---|---|---|---|---|---|---|
| | Upstream | Activation | p-value of | Upstream | Activation | p-value of |
| | Regulator | z-score | overlap | Regulator | z-score | overlap |
| Cluster 1 | STAT3 | 4.832 | 6.66E−18 | estrogen receptor | −3.646 | 1.18E−11 |
| | NFkB(complex) | 6.837 | 9.35E−15 | TRIM24 | −4 | 7.28E−09 |
| | IRF7 | 5.543 | 1.75E−10 | PPARA | −2.815 | 3.28E−05 |
| | JUN | 2.295 | 5.99E−10 | Hdac | −2.088 | 5.97E−05 |
| | STAT1 | 4.396 | 7.46E−10 | GATA3 | −2.566 | 1.49E−04 |
| | SP1 | 2.227 | 1.39E−09 | N-cor | −2.449 | 4.28E−04 |
| | TP63 | 3.434 | 1.95E−08 | PIAS4 | −2 | 2.57E−03 |
| | RELA | 2.793 | 2.23E−08 | KLF2 | −2.366 | 3.48E−03 |
| | HIF1A | 3.606 | 4.92E−07 | SPDEF | −2.931 | 4.92E−03 |
| | IRF3 | 2.82 | 5.77E−07 | MEOX2 | −2.646 | 1.54E−02 |
| Cluster 2 | TP53 (includes | 4.814 | 9.08E−17 | TBX2 | −4.69 | 1.92E−13 |
| | EG:22059) | | | | | |
| | CDKN2A | 4.748 | 3.78E−12 | FOXM1 | −2.797 | 4.04E−10 |
| | RB1 | 2.071 | 5.70E−09 | MYC | −4.208 | 8.37E−06 |
| | MYOCD | 3.366 | 9.94E−09 | SMAD7 | −2.704 | 8.55E−05 |
| | MKL1 | 2.956 | 7.52E−08 | E2F2 | −2.236 | 4.50E−04 |
| | TCF3 | 3.889 | 1.14E−07 | MYCN | −2.779 | 5.42E−04 |
| | SMARCB1 | 3.637 | 3.75E−06 | AHR | −2.85 | 8.86E−04 |
| | SRF | 3.847 | 5.29E−06 | HEY2 | −2.168 | 9.36E−04 |
| | HTT | 2.333 | 2.30E−05 | NFE2L2 | −2.707 | 4.29E−02 |
| | Rb | 2.425 | 1.80E−03 | SPDEF | −2.236 | 1.14E−01 |
| Cluster3 | AHR | 2.54 | 3.65E−12 | TP53 (includes | −3.296 | 2.27E−15 |
| | | | | EG:22059) | | |
| | estrogen receptor | 5.505 | 9.02E−12 | STAT3 | −4.084 | 3.15E−14 |
| | MYC | 3.71 | 1.10E−10 | SMARCA4 | −2.218 | 1.46E−11 |
| | SPDEF | 3.615 | 1.19E−08 | PGR | −2.175 | 2.35E−10 |
| | Hdac | 2.089 | 9.77E−08 | NFkB | −5.342 | 3.03E−10 |
| | SMAD7 | 3.504 | 2.40E−07 | STAT1 | −2.414 | 7.34E−10 |
| | PPARA | 3.246 | 7.64E−05 | HTT | −2.983 | 1.70E−08 |
| | TRIM24 | 3.742 | 5.93E−04 | SMAD3 | −3.87 | 5.92E−08 |
| | PPARG | 2.768 | 1.08E−03 | SRF | −4.105 | 7.32E−08 |
| | SREBF2 | 3.255 | 6.12E−03 | MKL1 | −2.96 | 3.79E−07 |

TABLE 3
Pathologic response to NAC at time of
cystectomy stratified by cluster number

| | Cluster 1 | | Cluster 2 | | Cluster 3 | |
|---|---|---|---|---|---|---|
| | N = 23 | | N = 26 | | N = 24 | |
| | N | % | N | % | N | % |
| Cystectomy | 15/23 | 65.2% | 25/26 | 96.2% | 17/24 | 70.8% |
| performed | | | | | | |
| NAC given | 5/15 | 33.3% | 7/25 | 28.0% | 6/24 | 25.0% |
| Pathologic | 3/5 | 60.0% | 0/7 | 0.0% | 4/6 | 66.7% |
| Response | | | | | | |
| No Response | 2/5 | 40.0% | 7/7 | 100.0% | 2/6 | 33.3% |
| Overall | 4/15 | 26.7% | 0/25 | 0.0% | 5/17 | 29.4% |
| Downstaged | | | | | | |
| Overall | 8/15 | 53.3% | 15/25 | 60.0% | 9/17 | 52.9% |
| Upstaged | | | | | | |
| No Change | 3/15 | 20.0% | 10/25 | 40.0% | 3/17 | 17.6% |
| in Stage | | | | | | |

Pathologic response/downstaging—decrease in stage to pT1/pT0 at cystectomy
Upstaged—increase to pathologic stage T3b or worse, or N+ at cystectomy
Chi-squared 7.34 degress of freedom 2p = 0.025
Null hypothesis: no relationship of cluster number and response to neoadjuvant chemotherapy
Alternate hypothesis: relationship between cluster number and response to neoadjuvant chemotherapy
With p = 0.025, the null hypothesis is rejected

TABLE 4A
Flash frozen cohort

| | Basal Cluster | Cluster 2 | Luminal Cluster | p-value |
|---|---|---|---|---|
| Cohort Size (n) | 23 (32%) | 26 (36%) | 24 (33%) | |
| Mean Age (y) ± SD | 70.1 ± 2.0 | 69.8 ± 1.7 | 66.4 ± 2.6 | 0.398 |
| Gender (n) | | | | |
| Male | 13 (57%) | 6 (23%) | 3 (13%) | 0.133 |
| Female | 10 (44%) | 20 (77%) | 21 (88%) | |
| Race (n) | | | | |
| Caucasian | 14 (61%) | 21 (81%) | 19 (79%) | |
| African American | 14 (61%) | 21 (81%) | 19 (79%) | 0.352 |
| Hispanic | 3 (13%) | 3 (12%) | 1 (4%) | |
| Clinical Stage at TUR (n) | | | | |
| ≦cT1 | 0 (0%) | 0 (0%) | 0 (0%) | 0.968 |
| cT2 | 13 (57%) | 16 (62%) | 13 (54%) | |
| cT3 | 7 (30%) | 8 (31%) | 8 (33%) | |
| cT4 | 3 (13%) | 2 (8%) | 3 (13%) | |
| Positive Clinical Lymph Nodes, cN+ (n) | 6 (26.1%) | 1 (3.8%) | 7 (29%) | 0.045 |
| Positive Clinical Metastasis, cM+ (n) | 5 (22%) | 0 (0%) | 2 (8%) | 0.035 |
| Clinical Stage Grouping (n) | | | | |
| Bladder Confined | 9 (39%) | 16 (62%) | 12 (50%) | |
| Locally Confined | 5 (22%) | 9 (35%) | 5 (21%) | 0.052 |
| Metastatic | 9 (39%) | 1 (4%) | 7 (29%) | |
| Lymphovascular Invasion at TUR (n) | 4 (17%) | 6 (23%) | 7 (29%) | 0.634 |
| Concurrent CIS | 7 (30%) | 13 (50%) | 10 (42%) | 0.380 |
| Primary Treatment (n) | | | | |
| Chemotherapy | 3 (13%) | 1 (4%) | 5 (21%) | |
| Cystectomy | 15 (65%) | 25 (96%) | 17 (71%) | |
| Other | 5 (22%) | 0 (0%) | 2 (8%) | |
| Neoadjuvant Chemotherapy (n) | 5 (22%) | 7 (27%) | 6 (25%) | 0.095 |
| Pathologic T stage (n) | | | | |
| pT0 | 2 (9%) | 0 (0%) | 2 (8%) | |
| pTa, pT1, pTis | 2 (9%) | 1 (4%) | 3 (13%) | |
| pT2 | 1 (4%) | 4 (15%) | 5 (21%) | |
| pT3 | 4 (17%) | 18 (69%) | 3 (13%) | |
| pT4 | 6 (26%) | 2 (8%) | 4 (17%) | |
| Positive Pathologic Lymph Nodes (n) | 3 (13%) | 14 (54%) | 6 (25%) | 0.010 |
| Positive Surgical Margin (n) | 3 (13%) | 3 (12%) | 1 (4%) | 0.056 |
| Variant histology at cystectomy | | | | |
| Squamous Differentiation | 5 (22%) | 2 (8%) | 0 (0%) | |
| Sarcomatiod | 3 (13%) | 1 (4%) | 0 (0%) | |
| Squamous Cell Carcinoma | 2 (9%) | 2 (8%) | 0 (0%) | 0.001 |
| Other (Micropapillary, Glandular, | 0 (0%) | 3 (12%) | 2 (8%) | |
| Adenocarcinoma) G E K | | | | |
| Median Overall Survival (m) | 14.9 | 34.6 | 65.6 | 0.098 |
| Median Disease Specific Survival (m) | 14.9 | Not Reached | 65.6 | .028 |

TABLE 4B
Korean database (one nearest neighbor test)

| | Basal Cluster | Cluster 2 | Luminal Cluster | p-value |
|---|---|---|---|---|
| Cohort Size (n) | 11 (20%) | 23 (42%) | 21 (38%) | |
| Mean Age (y) ± SD | 69.6 ± 8.4 | 61.5 ± 10.5 | 72.5 ± 7.1 | .001 |
| Gender (n) | | | | |
| Male | 6 (55%) | 21 (91%) | 15 (71%) | .049 |
| Female | 5 (46%) | 2 (9%) | 6 (29%) | |
| Clinical T Stage (n) | | | | |
| ≦cT1 | 0 (0%) | 0 (0%) | 1 (5%) | 0.755 |
| cT2 | 4 (36%) | 12 (52%) | 11 (52%) | |
| cT3 | 5 (46%) | 6 (26%) | 5 (24%) | |
| cT4 | 2 (18%) | 5 (22%) | 4 (19%) | |
| Positive Clinical Lymph Nodes - cN+ (n) | 4 (36%) | 6 (26%) | 5 (24%) | 0.740 |
| Positive Clinical Metastasis - cM+ (n) | 1 (9%) | 4 (17%) | 2 (10%) | 0.679 |
| Clinical Stage Grouping (n) | | | | |
| Bladder Confined | 3 (27%) | 10 (44%) | 9 (43%) | |
| Locally Confined | 4 (36%) | 6 (26%) | 7 (33%) | 0.863 |
| Metastatic | 5 (36%) | 7 (30%) | 5 (24%) | |
| Systemic Chemotherapy (n) | 5 (46%) | 13 (57%) | 7 (33%) | 0.304 |
| Median Overall Survival (m) | 10.4 | 26.4 | Not reached | 0.058 |
| Median Disease Specific Survival (m) | 11.2 | 66.3 | Not Reached | 0.102 |

### Discussion

Although recent studies have clearly established that bladder cancers are highly heterogeneous, the molecular underpinnings of this heterogeneity are still unclear. Here the inventors used GEP and unsupervised analyses to define the molecular heterogeneity in a cohort of muscle-invasive human bladder cancers. The inventors present evidence for the existence of three discrete molecular subsets of muscle-invasive cancer. Basal bladder cancers are characterized by active STAT3, p63, and NFκB signaling and express several canonical biomarkers of basal breast cancer (i.e., CD44, KRT5, KRT14, CDH3). They are enriched for squamous and sarcomatoid features and pathway analyses suggest that they are highly proliferative and motile. Like basal breast cancers, they are associated with particularly poor clinical outcomes, but paradoxically, and also like basal breast cancers, they are also highly sensitive to neoadjuvant chemotherapy. Importantly, other groups have independently determined that ΔNp63 and cytokeratins 5 and 14 identify tumors that are associated with poor clinical outcomes (Chan et al., 2009). Therefore, clinically applicable molecular diagnostic tests should be developed to detect basal bladder cancers at the time of diagnosis, and patients with these tumors should be treated aggressively with neoadjuvant chemotherapy. Because response to neoadjuvant chemotherapy is associated with excellent long-term survival, aggressive early management of basal bladder cancers offers the very realistic expectation of improved survival for patients with this form of bladder cancer.

The implication of STAT3 and p63 in the control of basal bladder cancer biology is consistent with developmental biological studies that have implicated p63 in the regulation of the basal layer of the normal urothelium and the inventor's previous work that identified ΔNp63 as a biomarker for the lethal subset of muscle-invasive bladder cancers. Using RNAi, the inventors confirmed that STAT3 and p63 both control the expression of several of the key molecular markers that identify basal tumors, including cytokeratins 5, 6 and 14, CD44, and P-cadherin (CDH3), and furthermore, that STAT3 functions upstream of ΔNp63 within this pathway.

As is true in the normal urothelium, it seems likely that the EGFR functions upstream of STAT3 and p63 to promote basal tumor biology. Components of the EGFR pathway (including the EGFR itself) were overexpressed in basal primary tumors, and EGFR inhibition resulted in downregulation of STAT3 phosphorylation and p63 expression in human bladder cancer cell lines. Furthermore, in previous studies the inventors showed that EGFR antagonists strongly inhibited proliferation in UM-UC5 (Shrader, Black), which was derived from a squamous (and therefore basal) primary tumor. Together, these observations support the idea that basal bladder cancers may be especially dependent upon autocrine EGFR signaling for their proliferation and/or survival. Importantly, however, UM-UC5 is the only cell line in the inventor's panel that exhibits high level EGFR gene amplification, and the other squamous cell line in this panel is only moderately sensitive to EGFR inhibitors. Aside from UC5, all of the other highly EGFR-dependent cell lines in the inventors' panel (UC4, UC7, UC9, and UC16) express relatively low levels of the basal markers CD44 and p63 and therefore appear to be luminal rather than basal. Finally, the clinical experience with EGFR inhibitors in bladder cancer has been disappointing, although previous clinical trials were performed without accounting for the molecular heterogeneity that is present in muscle-invasive cancers. Therefore, the biology of basal bladder cancer does provide a strong foundation for the further evaluation of EGFR inhibitors in carefully designed clinical trials in patients, particularly in tumors with high-level EGFR gene amplification and presumably in combination with conventional chemotherapy (since it is highly effective in the basal subset). However, given that there may be considerable redundancy with respect to the upstream signals that drive STAT3 activation, it may be more effective to target STAT3 directly.

There are also remarkable molecular similarities between luminal bladder and breast cancers. Luminal bladder cancers have gene expression profiles characteristic of active ER signaling and are characterized by the expression of several markers (CD24, KRT20, ERBB2, ERBB3, XBP1) that are shared by luminal breast cancers. The inventors attempted to study the role of the ER in controlling luminal gene expression in human bladder cancer cell lines, but we observed generally low levels of ERα and ERβ in all of our cell lines and RNAi-mediated modulation of their expression had relatively weak effects on differentiation-associated marker expression. The inventors attribute this to the general tendency of luminal bladder cells to acquire more basal characteristics after prolonged culture in vitro, a phenomenon that has also been observed with primary urothelial cells (Southgate). A high priority objective for our future studies will be to identify and/or develop better preclinical models of each of the bladder cancer subsets we have identified in this study.

The implication of the ER in luminal bladder cancer raises interesting questions about the possible impact of estrogens on its etiology, and preclinical and epidemiological data support the idea that they play important roles in cancer initiation and progression. The incidence of bladder cancer is at least 2-fold higher among men than it is in women, and the incidence of carcinogen (BBN)-induced tumors in mice is approximately 2-fold higher in males than it is in females. The normal urothelium expresses AR, ERα and ERβ, and recent studies have demonstrated that BBN does not induce tumors in AR knockout mice, strongly suggesting that the AR has tumor growth-promoting activities. On the other hand, mice with selective ERα knockout in the bladder mice are more susceptible to BBN-induced bladder tumors, suggesting that ERα signaling may exert a tumor suppressive function, perhaps by promoting urothelial differentiation. Because it appears that the basal and luminal transcriptional pathways antagonize each other (Southgate), it is possible that ER signaling inhibits the emergence of basal cancers by promoting luminal differentiation. The inventors attempted to study the role of the ER in controlling luminal gene expression in human bladder cancer cell lines, but we observed generally low levels of ERα and ERβ in all of our cell lines and RNAi-mediated modulation of their expression had relatively weak effects on differentiation-associated marker expression. The inventors attribute this to the general tendency of luminal bladder cells to acquire more basal characteristics after prolonged culture in vitro, a phenomenon that has also been observed with primary urothelial cells (Southgate).

On the other hand, PPAR signaling was an important feature of luminal tumor biology that appeared to be preserved better in human bladder cancer cell lines. We identified a subset of lines (UC1, SW780, UC14, RT4, RT112) that clustered together based on constitutive PPARγ pathway gene expression, and modulation of PPARγ signaling (via FABP4 knockdown) promoted upregulation of basal gene expression. Conversely, the PPARγ-specific ligand rosiglitazone induced expression of luminal markers (uroplakins, KRT8, KRT20) in the UC7 and UC9 that did not display strong baseline PPARγ pathway activation (FIG. 2). Strikingly, the presence of autocrine PPARγ signaling in the cell lines was also associated with high-level expression of FGFR3 and sensitivity to FGFR3 inhibitors, and the luminal subset of primary tumors was enriched for activating FGFR3 mutations. These observations raise the possibility that patients with luminal tumors will obtain the most benefit from FGFR inhibitor-based therapy. Again, since most luminal tumors respond to neoadjuvant cisplatin-based combination chemotherapy, the inventor's data support the development of FGFR inhibitor-based combinations rather than single agent approaches.

Pathway analyses of cluster 2 tumors revealed that they expressed gene expression profiles consistent with the presence of active p53 and other tumor suppressors (Rb, p16) (Table 2). Cluster 2 tumors did appear to contain fewer p53 mutations (5/21 evaluable) than either the basal (9/22) or luminal (9/22) subsets, suggesting that there may be some enrichment for wild-type p53 within cluster 2. However, there was also more missing data in cluster 2 (5 tumors) as compared to cluster 1 (1 missing) or 3 (2 missing), so this conclusion must be regarded as preliminary. In addition, recent preclinical studies indicate that epithelial tumors that retain a single copy of wild-type p53 are more similar to tumors that contain no p53 mutations than they are to tumors that contain one mutant p53 allele and display loss of heterozygosity (LOH) (deletion) of the other (Lozano). Therefore, while p53 mutational frequencies may provide some information about the relative importance of p53 mutations within our subsets, in the absence of LOH data the results must be interpreted as preliminary. On the other hand, studies in breast cancer have established that a p53-like gene expression profile accurately identified p53 mutant tumors over 80% of the time (Troester), and the p53 pathway can be disrupted even in bladder cancers that express wild-type p53, so a pathway approach may be a better way of identifying tumors with shared biology than direct p53 sequencing.

The most important characteristic of the “p53-like” cluster 2 tumors was that they tend to be resistant to neoadjuvant cisplatin-based chemotherapy. The efficacy of neoadjuvant chemotherapy is another similarity between breast and bladder cancers. The I-SPY trial (“Investigation of Serial Studies to Predict Your Therapeutic Response With Imaging and Molecular Analysis,” CALGB150007/150012) examined the correlation between pathological complete response rates (path CR, i.e., absence of residual tumor after presurgical therapy) and survival in women treated with neoadjuvant chemotherapy, and the results indicated that path CR was associated with substantially better rates of disease-specific and overall survival (Esserman JCO). These results prompted the FDA to enact a fast track path to approval for agents that produce high path CR rates in breast cancer in the neoadjuvant setting. Similarly, over 85% of bladder cancer patients whose tumors achieve a path CR following neoadjuvant cisplatin-based combination chemotherapy (MVAC or GC) are cured of their disease. Therefore, there is a strong push to make neoadjuvant chemotherapy the standard-of-care for bladder cancer patients who are going on to definitive surgical therapy (cystectomy), and a nationwide multicenter clinical trial that is very similar to I-SPY (called the “COXEN” trial) will be opening soon to determine whether biomarkers can be used to prospectively identify tumors that will be sensitive to neoadjuvant chemotherapy. In the I-SPY trial, tumors with either wild-type p53 or a gene expression profile consistent with the presence of wild-type p53 responded poorly to neoadjuvant chemotherapy. Preclinical studies have provided a mechanistic explanation for these findings: MMTV-driven murine breast cancers that retained at least one wild-type copy of p53 were able to undergo G1/S or G2 arrest in response to chemotherapy, whereas tumors that lacked wild-type p53 proceeded through mitosis and underwent apoptosis (Lozano). The inventor's results serve to reopen the discussion of the importance of p53 status in bladder cancer sensitivity to chemotherapy; an early report suggested that p53 mutant tumors would display enhanced sensitivity (Nature), but this conclusion was thought to be overturned in a recently completed, large recent multi-center clinical trial (JCO SWOG). We would argue that this issue can only be settled by performing a carefully designed, prospective study (like COXEN) to comprehensively measure p53 status and takes into account not only whether a mutation is present but also what the biological effects of that mutation might be and whether or not it is associated with LOH (i.e., complete absence of wild-type protein). In addition, given the number of ways the p53 pathway can be disrupted in tumors and the redundancy that is present within the p53 family itself (TA-p63 and TA-p73 share many of p53's effects), the presence of a wild-type p53 gene expression signature may be a more sensitive indicator of chemoresistance than p53 mutational status.

### Example 3

### ΔNp63αInhibits Epithelial-Mesenchymal Transition in Human Bladder Cancer Cells: Role of miR-205

Materials and Methodology

Cell Culture—

Cell lines were obtained from the MD Anderson Bladder SPORE Tissue Bank and cultured as in (Choi et al., 2012). Their identities were verified by DNA fingerprinting using AmpF1STR® Identifiler® Amplification (Applied Biosystems/Life Techonologies, Grand Island, N.Y.) or AmpF1STR® Profiler® PCR Amplification (Applied Biosystems/Life Techonologies) in the MD Anderson Characterized Cell Line Core facility.

Protein Overexpression and Gene Knockdown—

TAp63α (Open Biosystems/Thermo Scientific, Lafayette, Colo., EHS1001-7380111) and ΔNp63α(GeneCopoeia, Rockville, Md., EX-Z5740-MO2) were transfected into cells using Lipofectamine 2000 (Invitrogen/Life Techonologies, 11668-019) following the instructions provided by the manufacturer. The ΔNp63 specific siRNA (5′ ACAAUGCCCAGACUCAAUU 3′; SEQ ID NO: 1) was designed based on a previous publication (Chow et al., 2011) and was synthesized by Dharmacon/Thermo Scientific. The non-targeting siRNA was from Dharmacon (D-001810-10-20). siRNAs were transfected into cells using Lipofectamine RNAiMAX (Invitrogen/Life Techonologies, 13778-075). The panp63 lentiviral shRNA construct (V3LHS_{—}397885) that targets all p63 isoforms and the pGIPZ empty vector (RHS4339) were purchased from Open Biosystems. The ΔNp63αstable expression construct was cloned from the ΔNp63α-pReceiver-M02 expression vector (Genecopoeia, EX-Z5740-MO2) and packaged into a lentivirus. Pre-miR-205 vector was from System Bioscience (Mountain View, Calif., CD511B-1). Virus production, virus infection, and infected cell selection were performed in the MD Anderson Vector Core as described in (Marquis et al., 2012).

RNA Isolation and Real-Time Reverse Transcription PCR (qRT-PCR) Analysis—

RNA was isolated from cells using the mirVana™ miRNA Isolation Kit (Ambion/Life Techonologies). The AgPath-ID One-Step RT-PCR Kit (Applied Biosystems/Life Techonology) was used for real-time reverse transcription PCR. To qualify mature miRNAs, 10 ng of total RNA was reverse transcribed to cDNA using Taqman microRNA Reverse Transcription Kit (Applied Biosystems/Life Techonologies) and miRNA-specific primers. After that, real-time PCR was performed to measure mature miRNA expression. Gene expression was calculated by the comparative ΔΔCt method and displayed as relative quantity (RQ) ±RQ max and RQ min. Cyclophilin A was used as an endogenous control for mRNA expression and U6snRNA was the endogenous control for mature miRNA expression. Taqman primers and probes were obtained from Applied Biosystems. All PCR reactions were performed using either the ABI PRISM 7500 or the StepOne Plus PCR systems (ABI).

Flow Cytometry—

Cells were detached by 10 mM EDTA. One million cells were used for each immunoreaction. Blocking was performed in incubation buffer (0.5% bovine serum albumin—BSA—in PBS) for 15 minutes at room temperature. A direct staining method was employed for detection of N-cadherin using an allophycocyanin (APC)-conjugated anti-human N-cadherin antibody (R&D Systems, Minneapolis, Minn., FAB6426A) following the company's protocol. APC-conjugated sheep IgG was used as a negative control. Indirect staining was performed for P-cadherin using a polyclonal rabbit anti-P-cadherin antibody (Cell Signaling, Boston, Mass., 2130) and Alexa Fluor 594-conjugated goat anti-rabbit IgG (H+L) (Invitrogen/Life Technology, A11037) following a protocol from Cell Signaling. Negative control samples were stained with the secondary antibody alone.

Nuclear Run-on—

Experiments were performed as described in the short technical report (Patrone et al., 2000) with minor modifications. Briefly, cells were lysed on ice and nuclei were collected by centrifugation. Nuclei were then incubated with rATP, rCTP, rGTP (Epicenter Biotechnologies, Madison, Wis., RN02825) and Biotin-16-UTP (BU6105H) in transcription buffer for 30 minutes at 290 C. Reactions were then halted by adding a “stop” buffer containing 250 mM CaCl2, and 10 units/μl DNase I. RNA was purified and biotin labeled RNA was precipitated using magnetic beads coated with streptavidin (Dynabeads® M-280 Streptavidin, Invitrogen/Life Techonologies, 112.05D). High capacity cDNA reverse transcription kits (Applied Biosystems/Life Techonologies) were used to generate cDNA from the precipitated RNA, and qPCR was performed using the Fast SYBR Green master mix (Applied Biosystems/Life Techonologies).

Invasion Assays—

Cells were seeded into invasion inserts (UC6: 25×103 cells/insert, UC3: 15×103 cells/insert) of BD Biocoat™ Matrigel™ Invasion Chambers (BD Biosciences, San Jose, Calif., 354480) in triplicate. 3T3 conditioned medium was used as a chemoattractant. The chambers were incubated at 37° C. in a 5% CO₂ incubator for 48 hrs. After incubation, Matrigel membranes were fixed in 1% glutaraldehyde, and stained with gentian violet. Micrographs of the membranes were captured using an inverted microscope, and the numbers of invaded cells were counted using ImageJ software (Bethesda, Md.).

Immunoblotting—

Immunoblotting experiments were performed as described previously (Marquis et al., 2012). Primary antibodies used in this study were anti-panp63 (clone 4A4, Santa Cruz Biotechnology, Santa Cruz, Calif., sc-8431), anti-ZEB1 (Cell Signaling, 3396), anti-N-cadherin (Invitrogen/Life Technologies, 33-3900), and anti-Slug (Santa Cruz Biotechnology, sc-15391)

Chromatin-Immunoprecipitation (ChIP) Assay—

Experiments were performed using the ChIP-IT-Express kit from Active Motif (Carlsbad, Calif., 53009), according to the instructions from the manufacturer. For each ChIP reaction, we used 1-8 μg of antibody, either anti-panp63 (clone 4A4, Santa Cruz Biotechnology), anti-p53 (Millipore, Billirica, Mass., 17-613), anti-Pol II (Millipore, 17-620), or normal mouse IgG (Millipore, 12-371B). Precipitated DNA and the DNA input were amplified and analyzed by quantitative real-time PCR with SYBR green qPCR master mix (Applied Biosytems/Life Techonologies). Input DNA was used to normalize the values in each real-time PCR reaction. The relative enrichment of protein binding to target sequences is represented as RQ values (RQ=2−ΔCt×100; ΔCt=Ct(ChIP)−Ct(Input)). Real-time-PCR reactions were performed in triplicate and the results are presented as mean±SD for the triplicate samples. Data are representatives of two to three independent experiments.

Human Specimens—

Fresh frozen tumors from 98 patients obtained from the MD Anderson Genitourinary Cancer tissue bank were macrodissected to enrich for tumor content. Sample information and processing methods were described previously (Choi et al., 2012).

Statistical Methods—

The primary objectives were to examine correlations between p63 and miR-205 expression and to evaluate the association between marker expression and overall survival (OS) and disease-specific survival (DSS). Tumors at stage Ta or T1 were classified as superficial and stage ≧T2 tumors were considered as muscle-invasive. Correlations among expression of markers were quantified using Spearman's rho coefficients. The Kaplan-Meier estimate of survival distribution was displayed by the investigated biomarker expression characterized as high and low (e.g. p63, miR-205), where the cutoff point to define high and low was obtained from regression tree analyses. The log-rank test was used to compare survival distributions between groups. All p-values presented are 2-sided. p-values less than 0.05 were considered to be statistically significant. Statistical analyses were carried out using Splus 7 (Insightful Corp, Seattle, Wash.).

Results

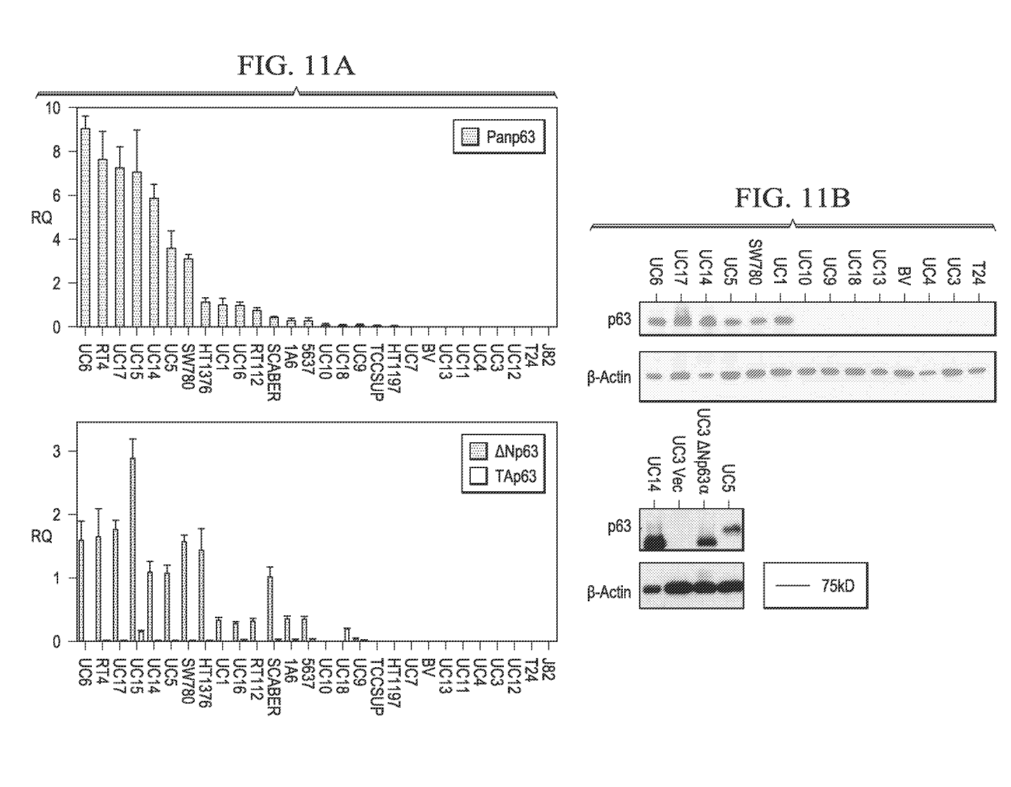

ΔNp63α is the most abundant isoform in human BC cell lines—Since p63 proteins exist as two groups of isoforms, TAp63 and ΔNp63, that potentially have different functions in cells, we compared their mRNA expression levels in a panel of human BC cell lines (n=28) using primers that detect all p63 isoforms (panp63) as well as TA and ΔN isoform-specific primers. The levels of ΔNp63 were substantially higher than the levels of TAp63 in the majority of the cell lines (FIG. 11A, right panel). Moreover, the patterns of panp63 and ΔNp63 expression were very similar (FIG. 11A, compare left and right panels), indicating that ΔNp63 is the most abundant mRNA isoform group in the BC cell lines. Immunoblot analyses of p63 protein expression, using the monoclonal mouse anti-human panp63 antibody 4A4 in a representative subset of 14 BC cell lines, revealed a strong band migrating at approximately 75 kD in all of the cell lines that expressed high ΔNp63 mRNA levels (FIG. 11B). Among the six p63 isoforms, TAp63α, TAp63β, and ΔNp63α are each approximately 75 kD in size (Roman et al., 2007). Because ΔNp63 was the most abundant isoform subgroup (FIG. 11A), the 75 kD immunoreactive band most likely corresponded to ΔNp63α. To more directly test this idea, TAp63a and ΔNp63α were overexpressed in a cell line with very low endogenous panp63 expression (UC3) and analyzed the expressed proteins by immunoblotting with 4A4. The results confirmed that the endogenous 75 kD immunoreactive band corresponded to ΔNp63α(FIG. 11 B).

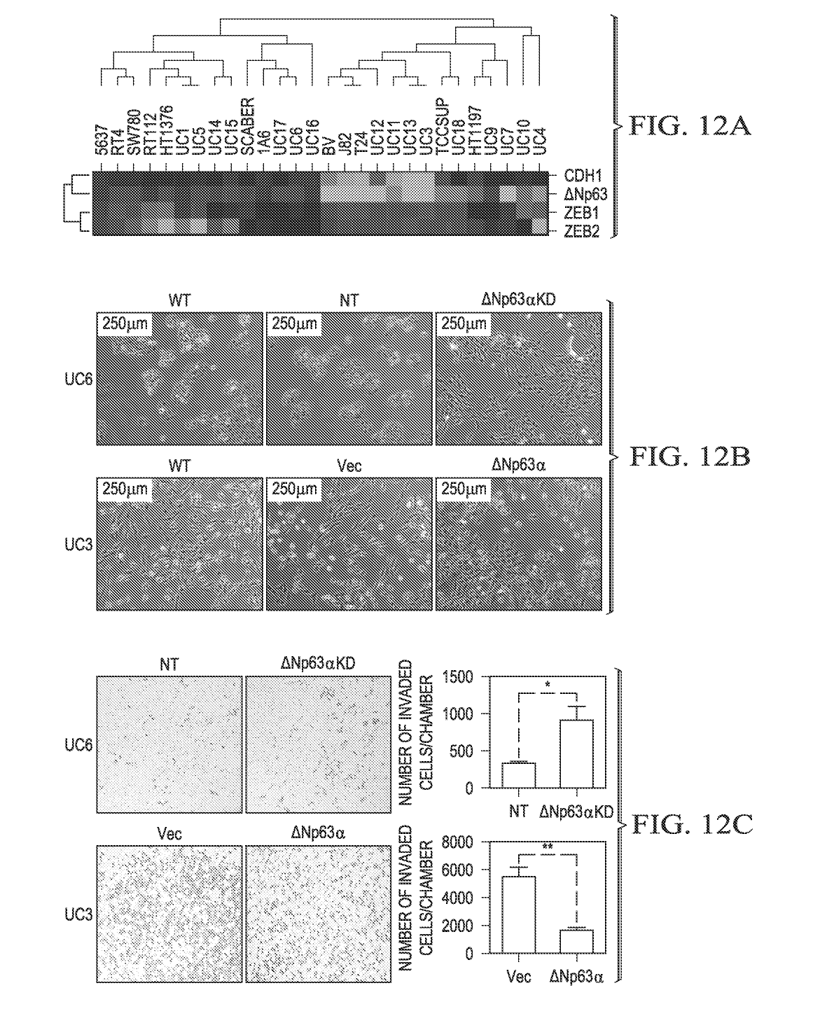

ΔNp63αsuppresses EMT—Previous studies showed that p63 isoforms play crucial roles in maintaining the stem cell compartments of epithelial tissues (Su et al., 2009; Senoo et al., 2007), and p63 directly regulates the expression of several “epithelial” markers, including cytokeratins (CKs) 5 and 14 and P-cadherin (Romano et al., 2009; Shimomura et al., 2008). Furthermore, it was recently reported that p63 and E-cadherin expression correlated closely with one another in human BC lines and primary tumors (Choi et al., 2012; Marquis et al., 2012). However, other recent work suggests that normal epithelial stem cells and cancer stem cells from epithelial tissues possess features of EMT (Mani et al., 2008). Therefore, the expression of epithelial and mesenchymal markers was first examined in the whole panel of BC cell lines (n=28) by qRT-PCR. Expression of ΔNp63 correlated closely with E-cadherin expression and correlated inversely with the expression of ZEB1 and ZEB2 (FIG. 12A).

A panp63 lentiviral shRNA construct was then used to stably knock down the expression of all p63 isoforms in UC6, a representative “epithelial” BC cell line that expresses high levels of ΔNp63 mRNA and protein. Because ΔNp63α is the most abundant p63 isoform in BC cells (FIG. 11), it was concluded that ΔNp63α is the primary isoform targeted by the panp63 shRNA construct. ΔNp63α was also overexpressed in UC3, a “mesenchymal” BC cell line that expresses low levels of all p63 isoforms at the RNA and protein levels (FIG. 11). Strikingly, the UC6 ΔNp63αKD cells exhibited morphological changes consistent with EMT, from displaying a characteristic “epithelial” polygonal appearance with discrete colonies to an elongated spindle-like shape, whereas the UC3 ΔNp63α overexpressing cells acquired morphological characteristics that resembled “epithelial” cells (FIG. 12B). Functionally, cells that have undergone EMT display increased invasion. Consistent with the effects of ΔNp63α modulation on cell morphology, the UC6 ΔNp63αKD cells exhibited increased invasion compared to the UC6 cells infected with a non-targeting construct, whereas the UC3 ΔNp63α-overexpressing cells became less invasive than the corresponding empty vector-infected controls (FIG. 12C).

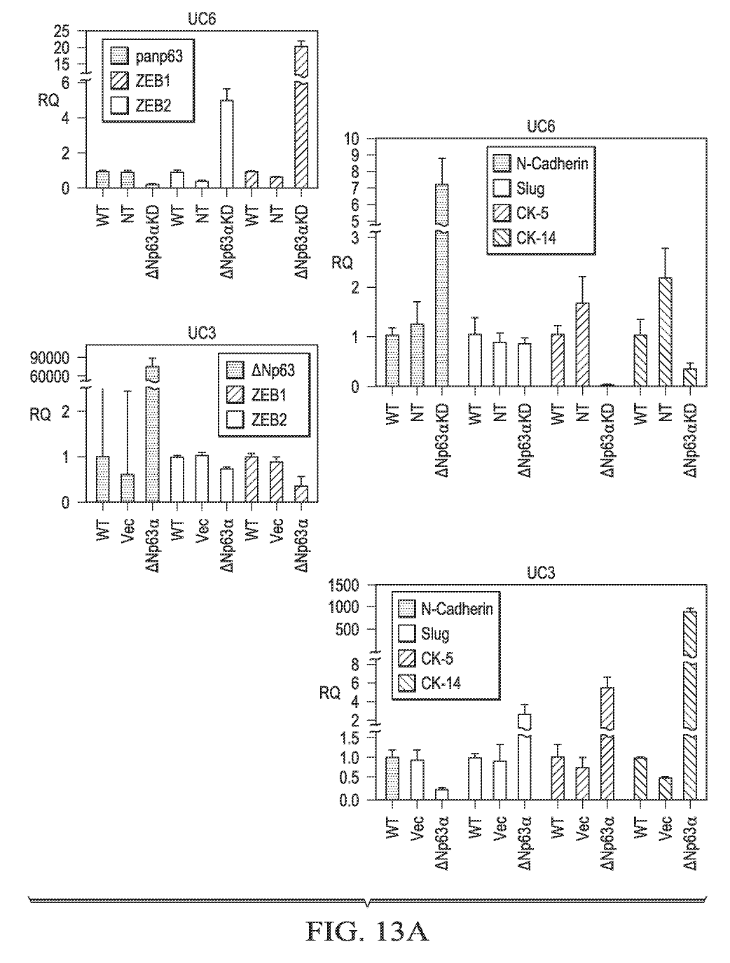

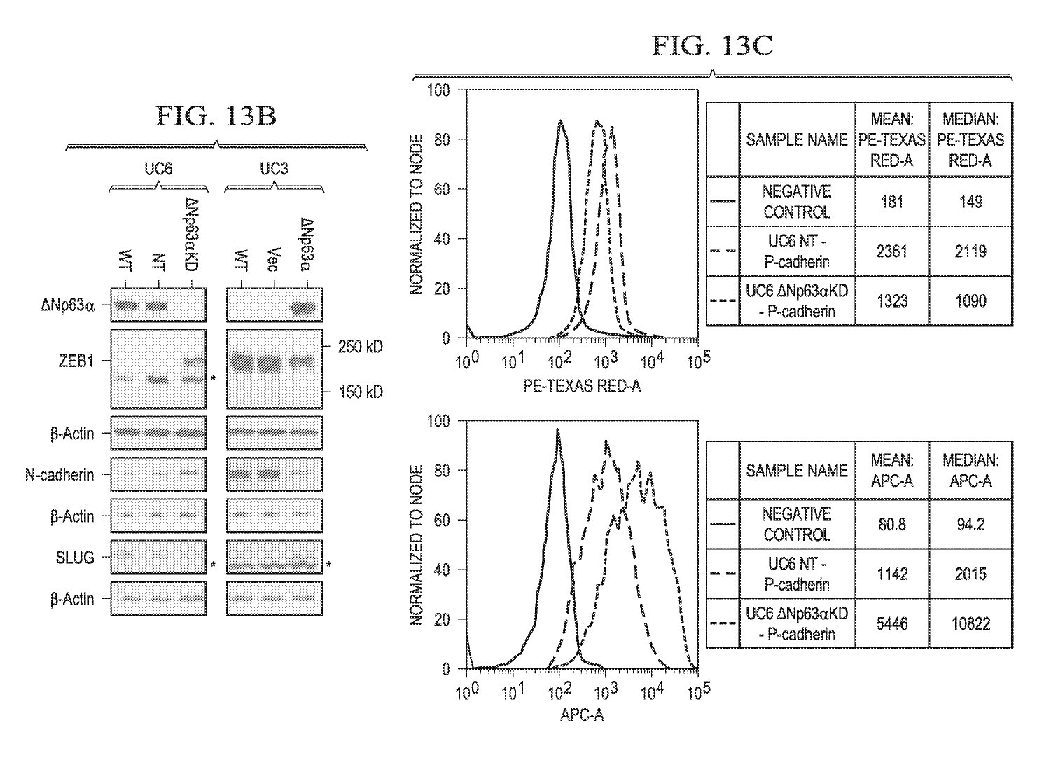

At the molecular level, EMT is characterized as the loss of epithelial markers and gain of mesenchymal markers. Therefore, qRT-PCR and/or immunobloting was performed to examine the effects of modulating ΔNp63α expression in the UC6 and UC3 cells on the expression of epithelial and mesenchymal markers. Interestingly, the levels of several mesenchymal markers (ZEB1, ZEB2, and N-cadherin) were significantly increased in the UC6 ΔNp63αKD cells and decreased in the UC3 ΔNp63α overexpressing cells, whereas expression of the epithelial markers CK-5 and CK-14 was decreased in the UC6 ΔNp63αKD cells and increased in the UC3 ΔNp63α overexpressing cells (FIGS. 13A and B).

Cadherins, a family of calcium dependent transmembrane glycoproteins, are major cell-cell adhesion molecules, playing important roles in development and carcinogenesis (Stemmler, 2008). P-cadherin is a basal cell-specific epithelial marker in the prostate and the bladder (Rieger-Christ et al., 2001; Jarrard et al., 1997). On the other hand, N-cadherin, the widely accepted mesenchymal marker (Lee et al., 2006), is absent in normal bladder mucosa but aberrantly expressed in bladder tumors. To more precisely define the effects of ΔNp63α modulation on EMT, surface P- and N-cadherin expression was measured by two-color surface staining and flow cytometry (FACS) (FIG. 13C). The results demonstrated that the UC6NT cells were double positive for P- and N-cadherin, consistent with “partial EMT” (Tsai et al, 2012) at baseline (FIG. 13C). The UC6 ΔNp63αKD exhibited reduced expression of P-cadherin and increased expression of N-cadherin, and a new population of cells emerged (approximately 50% of the total) that were N-cadherin positive but P-cadherin-negative (data not shown). These analyses demonstrate that ΔNp63αKD modulated the functionally relevant (surface) pools of P- and N-cadherin in the UC6 cells and that they were modulated across the entire cell population.

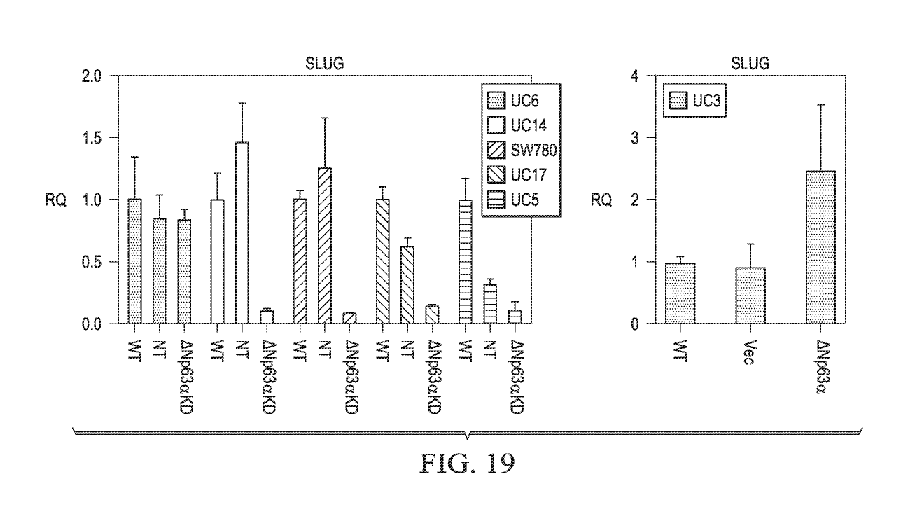

Slug (SNAI2) was the only EMT-related marker that did not conform to this pattern: expression of Slug was decreased by ΔNp63αKD in all of the cell lines we examined and was increased in the UC3 cells transduced with ΔNp63α(FIG. 13A,B and FIG. 19). This observation indicates that ΔNp63αpromote some mesenchymal characteristics and may help to explain the “partial EMT” (Tsai et al., 2012) phenotype that is observed in the parental UC6 cells at baseline.

ΔNp63αExpression Correlates with miR-205 Expression in BC Cell Lines and BC Primary Tumors—

ZEB1 and ZEB2 are canonical EMT markers that function to directly suppress E-cadherin expression (Comijn et al., 2001; Eger et al, 2005.). The close correlation between ΔNp63 and E-cadherin expression as well as the inverse correlation between ΔNp63 and ZEB1/2 drew our interest to the possible relationship between ΔNp63 and ZEB1/2. Because p63 interacts with p53 response elements (p53REs) (Westfall and Pietenpol, 2004), first p53REs were searched for in the ZEB1 and ZEB2 promoters but none were identified, suggesting that ΔNp63α does not control expression of ZEB 1 and ZEB2 directly. Gene expression profiling was then used (Illumina HT12V4 chips) to identify all of the EMT-related changes induced by ΔNp63αKD in triplicate RNA isolates obtained from UC6 and another p63-positive BC line (UC14), cells transduced with the non-targeting lentiviral vector, and cells transduced with the panp63 shRNA construct. One of the most striking and consistent alterations was down-regulation of the primary form of miR-205 (data not shown), a known direct inhibitor of ZEB1 and ZEB2 Gregory et al., 2008a; Gregory et al., 2008b).

A recent study concluded that p53 also inhibits EMT by regulating the expression of miR-200c (Chang et al., 2011). Therefore, expression of the 5 members of the miR-200 family was measured in the isolates, but down regulation of miR-200c or any of the other family members was not observed in either cell line.

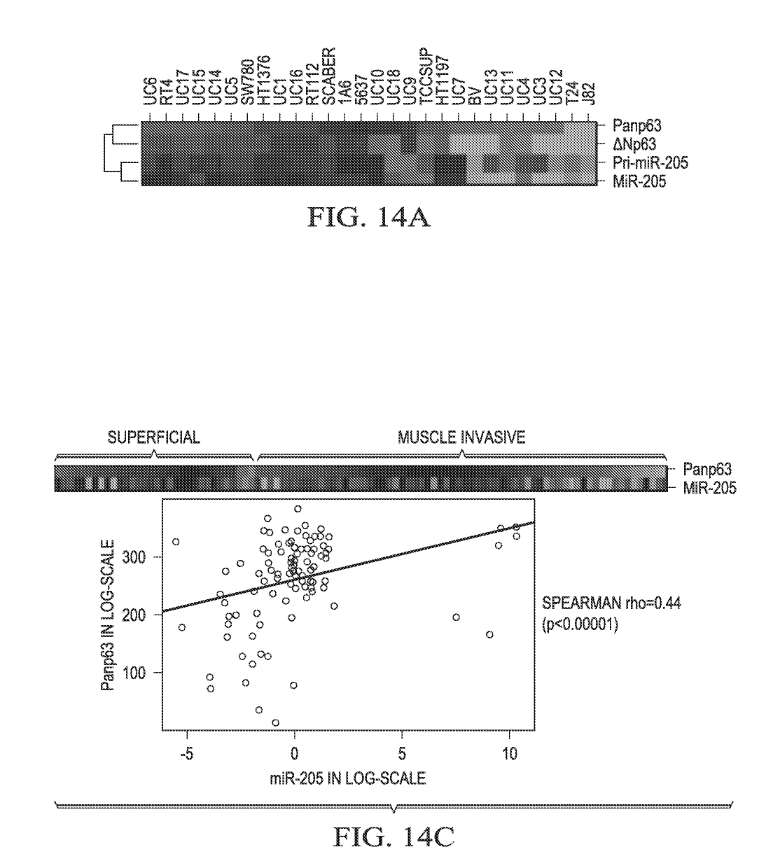

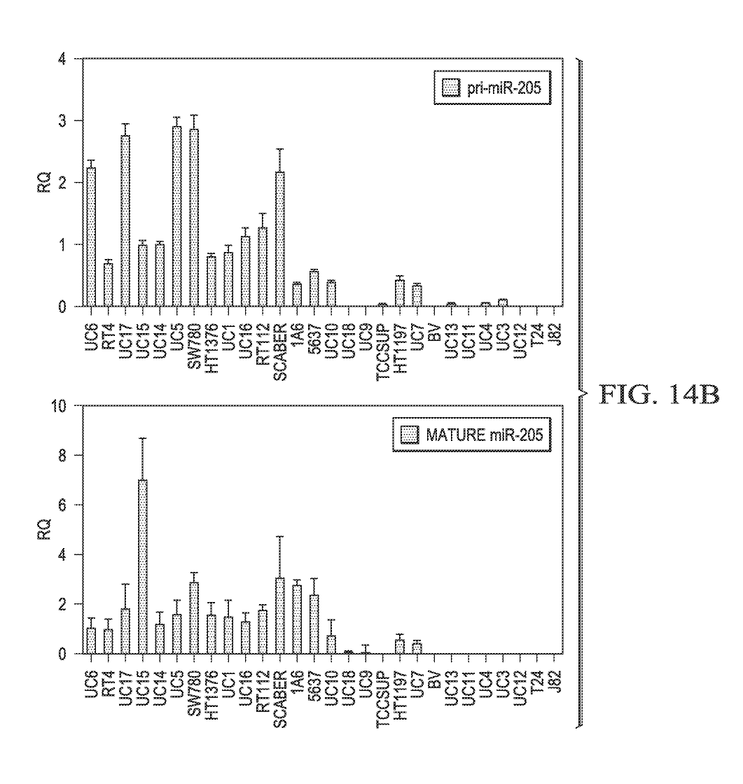

To confirm our gene expression profiling data, we performed qRT-PCR using primers for panp63, ΔNp63, the primary form of miR-205 (pri-miR-205) and the mature form of miR-205 (miR-205) in RNA isolated from the 28 BC cell lines in our panel. Statistical analyses revealed a strong correlation among the expression levels of these markers (Spearman rho≧0.79, p<0.0001) (FIGS. 14A and B and Table 5). The close correlation between the expression of the primary and mature forms of miR-205 in the majority of the cell lines suggests that transcription rather than miRNA processing plays a central role in maintaining mature miR-205.

TABLE 5
Statistical analysis using Spearman method to analyze
the correlation of panp63, ΔNp63, TAp63, pri-
miR205, miR205 expression in BC cell lines.

| Spearman rho | | | Pri-miR- | |
|---|---|---|---|---|
| (p-value) | panp63.RQ | ΔNp63.RQ | 205.RQ | miR-205.RQ |
| panp63.RQ | 1 | 0.93(p < | 0.85(p < | 0.79(p < |
| | | 0.0001) | 0.0001) | 0.0001) |
| ΔNp63.RQ | | 1 | 0.83(p < | 0.85(p < |
| | | | 0.0001) | 0.0001) |
| Pri-miR- | | | 1 | 0.82(p < |
| 205.RQ | | | | 0.0001) |
| miR205.RQ | | | | 1 |

The expression of panp63 and mature miR-205 was also compared in a cohort of 32 superficial and 66 muscle-invasive primary BCs from patients. Again, the results indicated that a close correlation existed between the two (Spearman rho=0.44, p<0.00001) (FIG. 14C). Since ΔNp63α is the major isoform present in BC cell lines (FIG. 11) and BC primary tumors (19,37), the results support the data obtained from the gene expression profiling studies implicating ΔNp63α in the regulation of miR-205 expression.

ΔNp63αRegulates ZEB1/2 by Modulating miR-205—

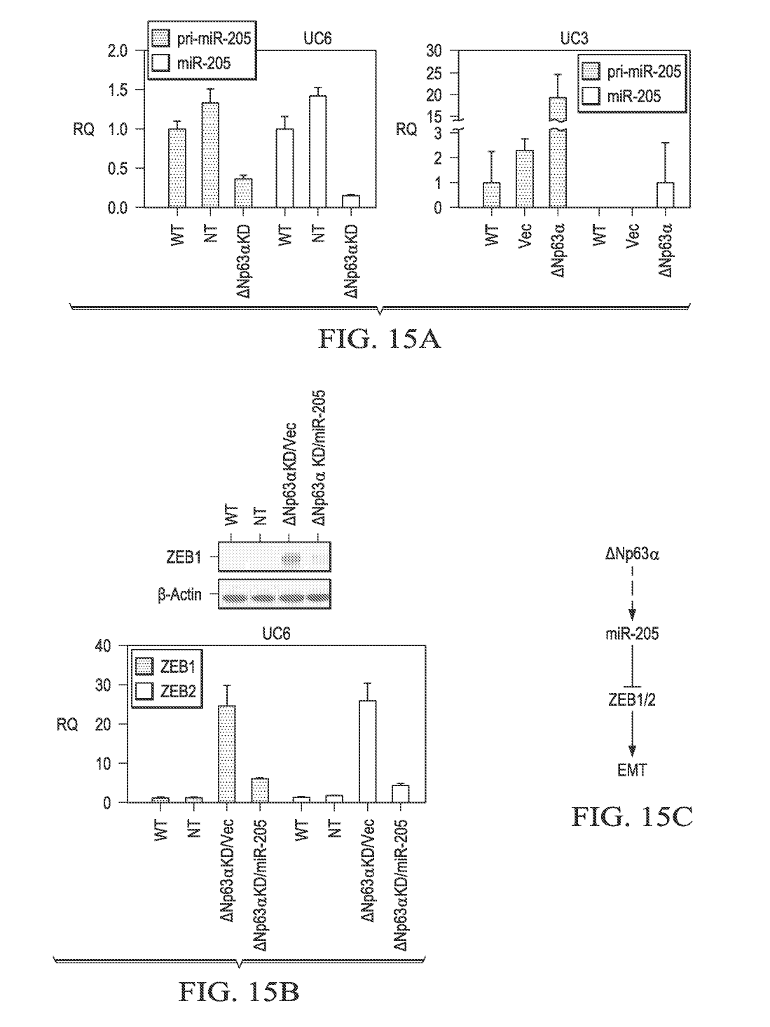

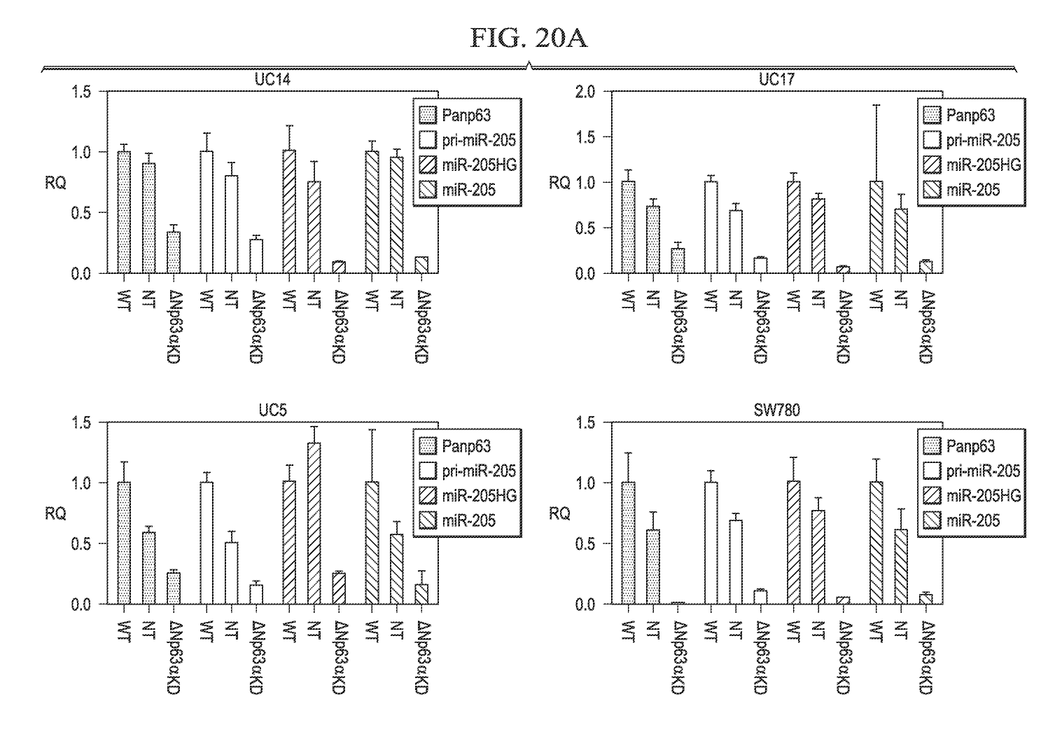

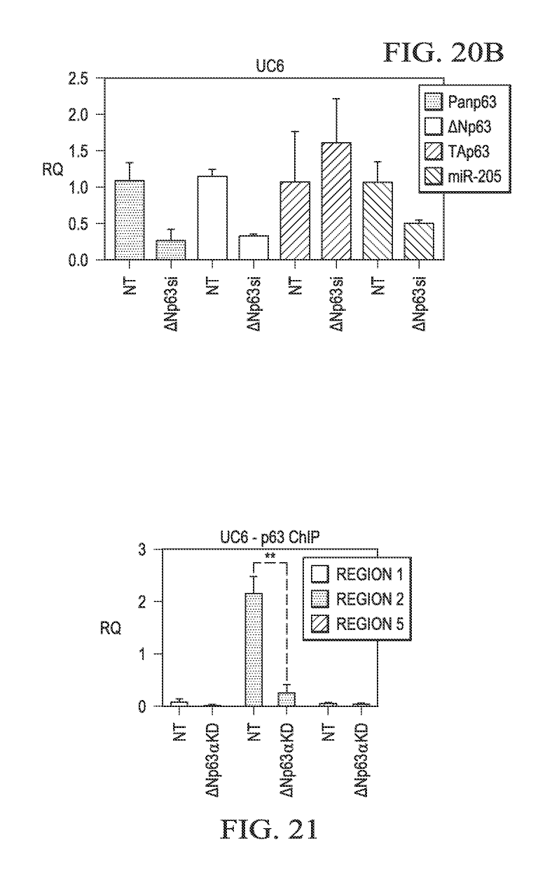

To further examine the relationship between ΔNp63α and miR-205, we used quantitative RT-PCR to measure the primary and mature forms of miR-205 in the UC6 ΔNp63αKD and UC3 ΔNp63α overexpressing cells. Consistent with the gene expression profiling data, ΔNp63αKD in UC6 decreased the expression of both primary and mature forms of miR-205, whereas overexpression of ΔNp63α in UC3 resulted in the opposite effects, indicating that ΔNp63αdirectly or indirectly modulated miR-205 expression (FIG. 15A). These results were confirmed in four additional “epithelial” BC lines (UC14, UC17, UC5 and SW780) (FIG. 20A). The results were also confirmed using an independent, ΔNp63-specific siRNA, which also decreased miR-205 expression in the UC6 cells (FIG. 20B). To determine whether decreased miR-205 expression mediates the effect of ΔNp63αKD on ZEB1 and ZEB2 expression, we overexpressed miR-205 in the UC6 ΔNp63αKD cells. Overexpression of exogenous miR-205 largely reversed the increased ZEB1 and ZEB2 expression induced by ΔNp63αKD (FIG. 15B), confirming that decreased miR-205 expression plays an important role in the response. The relationship between ΔNp63α and EMT is summarized in FIG. 15C.

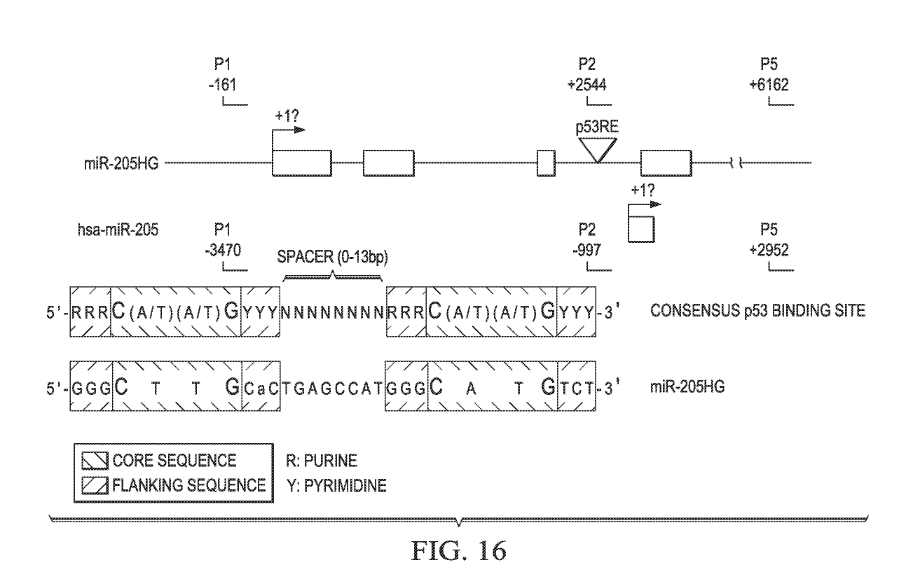

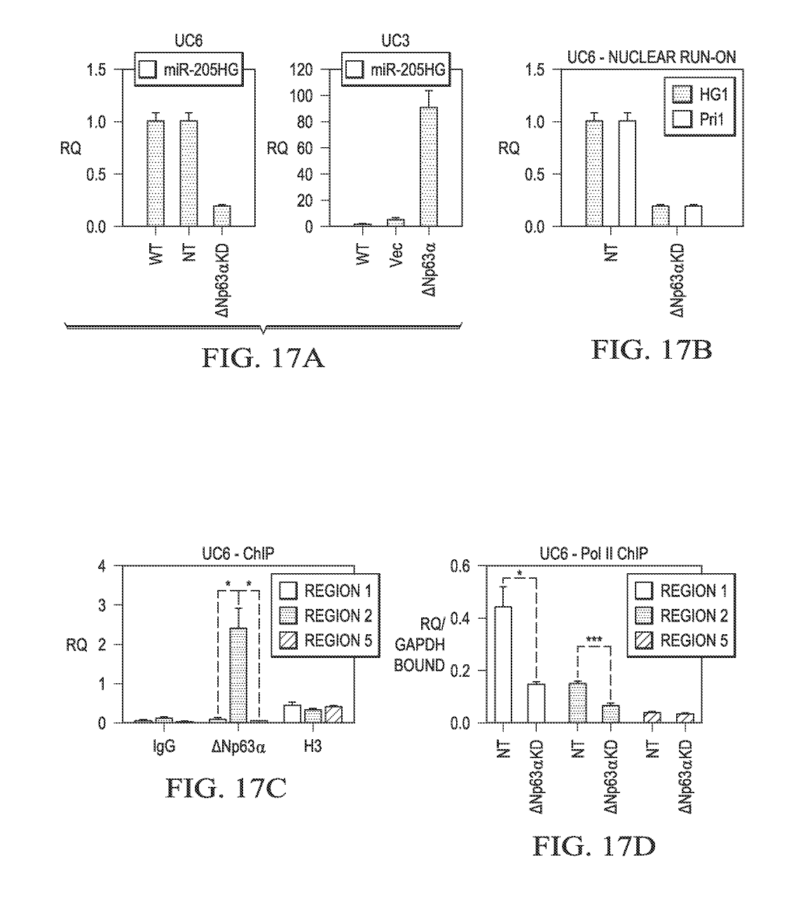

MiR-205 is regulated via its “host” gene—Genomic localization analyses of miRNAs indicates that they can be grouped into two classes, intergenic miRNAs and intragenic miRNAs. Intergenic miRNAs are located between genes and are controlled as independent transcriptional units. Intragenic miRNAs are located within annotated genes which are considered the “host” genes for the miRNAs (Saini et al., 2007). The transcription patterns of intragenic miRNAs and their “host” genes suggest that this class of miRNAs is transcribed in parallel with their “host” genes (Rodriguez et al., 2004; Baskerville and Bartel, 2005). The genomic location of miR-205 overlaps the junction between the last intron and the last exon of a “host” gene that has been termed miR-205HG (miR-205 “host” gene), formerly known as LOC642587. MiR-205HG is a protein coding gene that contains four exons and three introns (FIG. 16A). We performed quantitative RT-PCR using primers hybridizing to the exon 2 and 3 junctions of miR-205HG to determine the effects of ΔNp63α knockdown or overexpression on miR-205HG expression. The results showed that the expression of miR-205HG was changed in parallel with miR-205 when ΔNp63α expression was modified (FIG. 17A and FIG. 20A). The data confirm that there is a link between expression of miR-205 and its “host” gene and that expression of both is coordinated by ΔNp63α.

Steady state mRNA levels are controlled by a balance between transcription and RNA degradation. To determine the role of ΔNp63α in the transcriptional control of miR-205 and miR-205HG, we performed nuclear run-on experiments using biotin-labeled dUTP. This technique allowed us to directly measure the rates of transcription for miR-205HG and pri-miR-205 by real-time PCR. The rates of transcription for both pri-miR-205 and miR-205HG were decreased by over 50% in the UC6 ΔNp63αKD cells compared to those observed in the NT cells (FIG. 17B).

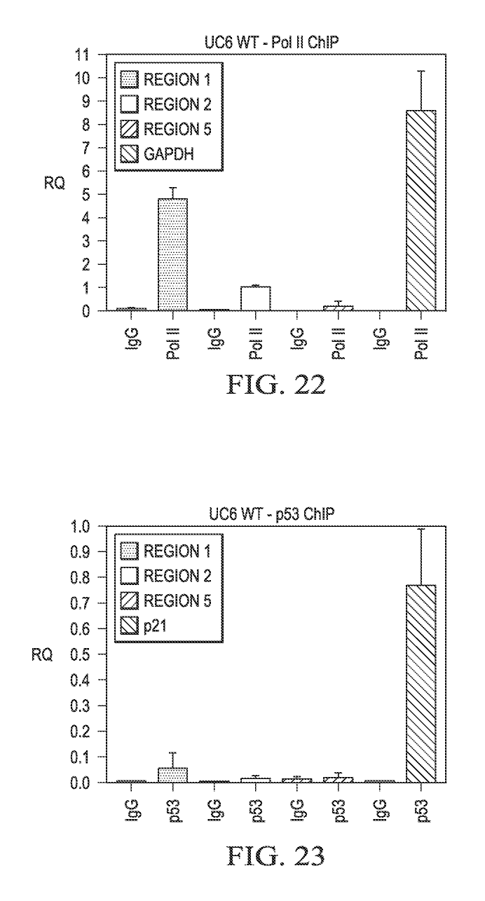

Even though intragenic miRNAs may be transcribed together with their host genes, some reports have concluded that intragenic miRNAs can also have their own promoters and be transcribed independently (Ozsolak et al., 2008; Corcoran et al., 2009). Analysis of the 1 kb region upstream of the miR-205 start site using the UCSC Genome Browser (available of the world wide web at genome.ucsc.edu) revealed a region that was highly conserved across 46 different vertebrate species (region 2), similar to the promoter region of miR-205HG which is the 1 kb region upstream of the first exon (region 1) (FIG. 16B). Moreover, region 2 is also hypersensitive to DNaseI (FIG. 16B), indicating its likely role as a regulatory region or functional promoter. Intriguingly, a p53RE was identified within region 2 that was also detected by Genomatix (FIGS. 16A,B). A p53 response element generally contains two tandem copies of a 10 bp sequence homologous to the consensus binding motif 5′ PuPuPuC(A/T)(A/T)GPyPyPy 3′, separated by a 0-13 bp spacer (el-Deirv et al., 1992). Each binding motif, which is comprised of a core sequence (C(A/T)(A/T)G) and each of the two flanking sequences (PuPuPu and PyPyPy), is considered to be a half-site of the p53RE. The p53RE identified in region 2 is a canonical whole-site p53RE with only one mismatch in the flanking sequence (FIG. 16A). However, there were no canonical p53REs within the proximal promoter of the miR-205HG. Chromatin immunoprecipitation (ChIP) using primers specific for region 1, region 2 or an intronic region 2.5 kb away from the last exon of miR-205HG (region 5) confirmed that ΔNp63αonly binds to region 2 (FIG. 17C). The binding of ΔNp63α to region 2 was reduced in the UC6 ΔNp63αKD cells, indicating that the binding was specific (FIG. 21). To determine whether region 1 or region 2 is the promoter for miR-205, we performed ChIP using an anti-RNA Pol II antibody. Strong enrichment of Pol II binding at region 1 and less binding at region 2 was observed, strongly suggesting that region 1 serves as the promoter for both miR-205HG and miR-205 (FIG. 22). More importantly, ΔNp63αKD significantly reduced the binding of Pol II at region 1 and region 2, demonstrating the importance of ΔNp63α in Pol II recruitment to miR-205 (FIG. 17D).

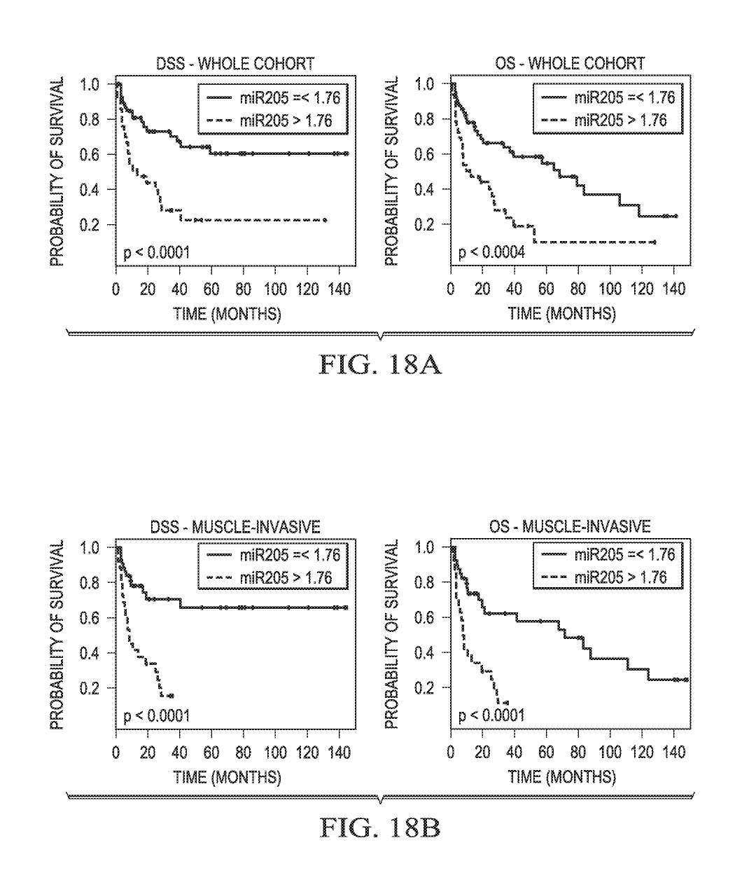

High miR-205 expression correlates with adverse clinical outcomes—Recent studies concluded that high ΔNp63 expression correlates with unfavorable clinical outcomes in patients with MIBC (Karni-Schmidt et al., 2011; Choi et al., 2012). Given that it is a downstream transcriptional target of ΔNp63, we wondered whether miR-205 might also serve as a biomarker for the lethal BC subset. Regression tree analyses were used to determine the cutoff point of miR-205 expression as 1.76 within our dataset. In the whole cohort of tumors (superficial plus muscle invasive), elevated expression of miR-205 was associated with a median disease-specific survival (DSS) of 13.4 months and a median overall survival (OS) of 12 months, while low miR-205 expression was associated with a significantly better median DSS of 140+ months and median OS of 69.1 months (p<0.0001 for DSS and p=0.0004 for OS) (FIG. 18A). When we confined the analyses to the MIBC subgroup, the association between high miR-205 expression and adverse clinical outcome was even more significant. Patients whose MIBCs expressed high miR-205 levels had a median DSS and OS of only 8.11 months, whereas those with tumors that expressed low miR-205 levels also had a median DSS of 140+ months and OS of 69.1 months (p<0.0001 for DSS and p<0.0001 for OS) (FIG. 18B). Therefore, like ΔNp63, high miR-205 expression identifies the lethal BC subset.

### Discussion

Our data implicate ΔNp63α, the most abundant isoform of p63 expressed in BC, in the control of EMT. We also show for the first time that ΔNp63α binds to a highly conserved regulatory region upstream of the miR-205 start site, participates in the recruitment of RNA Pol II to the promoter of the miR-205 host gene (miR-205HG), and coordinates the transcription of both miR-205HG and miR-205. miR-205 transcriptional regulation is one mechanism by which ΔNp63αcontrols EMT, because up- or down-regulation of ΔNp63αresults in parallel changes in miR-205 levels and reciprocal effects on the canonical EMT inducers, ZEB1 and ZEB2. However, these results also show that ΔNp63αcontrols the expression of several other EMT-related targets, and we do not think that they are all directly or indirectly regulated via miR-205. Therefore, future studies should be designed to identify the molecular mechanisms involved in these other EMT-related effects of ΔNp63α.

Interestingly, ΔNp63α has been shown to promote TGFβ-induced EMT in normal human keratinocytes (Oh et al., 2011). The data herein demonstrate that ΔNp63αpromotes the expression of at least one important mesenchymal marker in BC cells, Slug (SNAI2), consistent with the conclusion that ΔNp63α has some EMT-promoting effects. These data also show that the parental UC6 cells are not purely epithelial but exhibit a “partial EMT” phenotype at baseline (Tsai et al., 2012). However, the overall EMT-promoting impact of ΔNp63α-dependent Slug expression on cellular morphology and invasion appears to be outweighed by ΔNp63α's suppressive effects on EMT in tested cell lines.

Because the domain (region 2) that physically interacts with ΔNp63αcontains a whole-site p53RE, it is possible that p53 and p73 also interact with this region. Indeed, a previous paper concluded that p53 binds to region 2 and controls the expression of miR-205 in breast cancer (Piovan et al., 2012). ChIP experiments were performed to directly test this possibility but did not observe any enrichment of p53 binding at region 2 in the UC6 cells, which express wild-type p53 (FIG. 23). Furthermore, there was no correlation between the mutational status of p53 and the expression of miR-205 (or for that matter members of the miR-200 family) in our BC cell lines (Sabichi et al., 2006), suggesting that p53 is not centrally involved in maintaining expression of these “epithelial” micro RNAs in BC cells. Importantly, our conclusions regarding the importance of ΔNp63α in regulating miR-205 expression are consistent with recent work in prostate cancer cells (Gandellini et al., 2012).

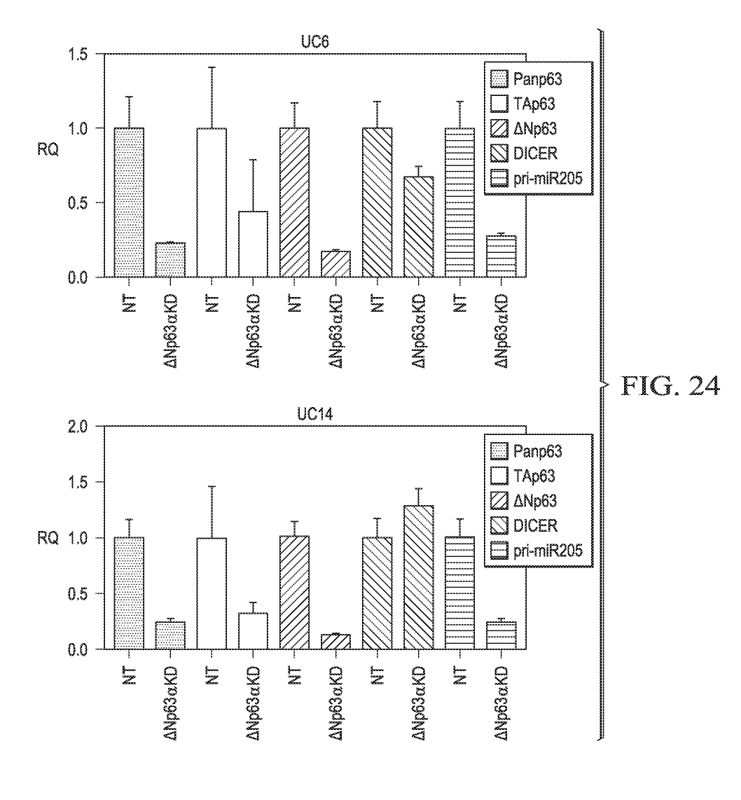

TAp63 plays a crucial role in suppressing metastasis via regulation of Dicer expression, which leads to downstream global effects on micro RNA expression (Su et al., 2010). In our BC cell lines, the panp63 shRNA produced no changes in Dicer mRNA expression (FIG. 24), and in fact it actually led to increased miR-200c expression in the UC14 cells (data not shown). Given that our BC cells generally expressed very low levels of TAp63 and the effects of ΔNp63α were associated with increased miR-205HG and miR-205 transcription, these observations are not surprising and do not contradict previous findings (Su et al., 2010).

Similar to the majority of intragenic miRNAs, miR-205 is transcriptionally co-regulated with its “host” gene, and ΔNp63α is somehow critical for this regulation. However, exactly, how ΔNp63αpromotes Pol II recruitment to miR-205HG promoter remains unresolved. In contrast to clear binding of ΔNp63α to region 2, the ChIP results indicate that ΔNp63α does not interact directly with the miR-205HG proximal promoter. These negative results do not rule out the possibility that region 2 serves as a downstream enhancer or that ΔNp63α binds to an unidentified distal miR-205HG enhancer element. However, the fact that ΔNp63αlacks a full-length N-terminal transcriptional transactivation domain, generally associated with direct regulation of transcription, suggests that a different mechanism is probably involved.

ΔNp63 and its downstream target, miR-205, are markers of the “epithelial” phenotype. p63 is uniformly expressed in the basal layer of the normal urothelium which contains urothelial stem cells (Kurzrock et al., 2008) and in superficial BC, which is usually low grade and non-lethal (Karni-Schmidt et al., 2011). We have reported a correlation between elevated expression of ΔNp63 and adverse outcomes in patients with MIBCs (Choi et al., 2012). In this study, we observed that high miR-205 expression also correlates with poor outcomes in MIBC patients. Our conclusion that ΔNp63αcoordinates the expression of multiple genes in BC cells to reinforce the “epithelial” phenotype and at the same time is associated with poor clinical outcomes in patients may seem somewhat paradoxical given that EMT is considered essential for tumor metastasis (McConkey et al., 2009), and metastasis is invariably associated with BC mortality. Our observation that ΔNp63αpromotes the expression of Slug and therefore a “partial EMT” phenotype (Tsai et al., 2012), coupled with the fact that ΔNp63 controls the expression of BC stem cell markers (including CK-5 and CK-14) (Volkmer et al., 2012) helps to resolve this paradox. Furthermore, emerging evidence indicates that EMT “plasticity” is crucial for productive metastasis (Tsai et al., 2012; Polyak et al., 2009). Even though preclinical studies have clearly established the importance of EMT in metastasis, tumor metastases in patients express epithelial markers (Chao et al., 2010; Hugo et al., 2007), which has raised doubts about the relevance of the preclinical observations to the process of tumor metastasis in patients. However, a recent study provides an elegant resolution to this apparent contradiction. Using an inducible Twist expression construct in a popular mouse model of carcinogen-induced head and neck squamous cell carcinoma (HNSCC), the authors demonstrated that primary tumors use EMT to escape from the primary tumor, form circulating tumor cells (CTCs), and extravasate into lymph nodes and distant organs, but they remain dormant unless they subsequently undergo “mesenchymal-to-epithelial transition” (MET), which facilitates proliferation. Importantly, the CTCs in this model and in patients still express epithelial cytokeratins, indicating that the process involves a “partial EMT” (Tsai et al., 2012). Therefore, it is possible that ΔNp63α expression is dynamically regulated during this process and that the cells in transit (i.e., circulating tumor cells, CTCs) actually express lower levels of ΔNp63α than do cells within the primary tumor or metastases.

Although miR205 expression is associated with a lethal BC phenotype, this does not necessarily mean that miR205 drives lethal biology. Instead, it appears that miR205 is associated with poor outcomes because it is a marker of ΔNp63 activity. Support for this conclusion comes from an ongoing study where we are using unsupervised hierarchical clustering of gene expression profiling data from MIBCs to determine whether discrete biological subsets exist within them (as has been demonstrated in breast cancers) (Perou et al., 2000). We have identified 3 discrete subsets within our MIBCs and in three other independent gene expression profiling datasets. Ingenuity pathway analyses revealed that BCs within the subset that is associated with the worst clinical outcomes is enriched for expression of Np63 downstream targets, including P-cadherin, CK-5 and CK-14 (W. Choi, manuscript in preparation). These cancers may possess a “basal” phenotype because they arose via neoplastic transformation of normal urothelial basal stem cells, whereas the other two subsets appear to have evolved from independent, more well-differentiated “luminal” progenitors. Our results are also consistent with other recent work that identified CK-5 and CK-14 as markers of BC stem cells and poor outcomes in other cohorts of MIBCs (Volkmer et al., 2012; Chan et al., 2009).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
International (PCT) Patent Application No. PCT/US2011/026329
U.S. Patent Publn. 2013/0084241
Sanchez-Carbayo et al., (2006) Defining molecular profiles of poor outcome in patients with invasive bladder cancer using oligonucleotide microarrays. J Clin Oncol 24: 778-789.
Blaveri et al., (2005) Bladder cancer outcome and subtype classification by gene expression. Clin Cancer Res 11: 4044-4055.
Dyrskjot et al., (2003) Identifying distinct classes of bladder carcinoma using microarrays. Nat Genet 33: 90-96.
Sjodahl et al., (2012) A molecular taxonomy for urothelial carcinoma. Clin Cancer Res 18: 3377-3386.
Lee et al., (2010) Expression signature of E2F1 and its associated genes predict superficial to invasive progression of bladder tumors. J Clin Oncol 28: 2660-2667.
Kim et al., (2010) Predictive value of progression-related gene classifier in primary non-muscle invasive bladder cancer. Mol Cancer 9: 3.
Wright et al., (2003) A random variance model for detection of differential gene expression in small microarray experiments. Bioinformatics 19: 2448-2455.
Eisen et al., (1998) Cluster analysis and display of genome-wide expression patterns. Proc Natl Acad Sci USA 95: 14863-14868.
Livak et al., (2001) Analysis of relative gene expression data using real-time quantitative PCR and the 2(-Delta Delta C(T)) Method. Methods 25: 402-408.
Cote et al., (1998) Elevated and absent pRb expression is associated with bladder cancer progression and has cooperative effects with p53. Cancer Res 58: 1090-1094.
Choi et al., (2012) p63 expression defines a lethal subset of muscle-invasive bladder cancers. PLoS One 7: e30206.
Karni-Schmidt et al., (2011) Distinct expression profiles of p63 variants during urothelial development and bladder cancer progression. Am J Pathol 178: 1350-1360.
Perou et al., (2000) Molecular portraits of human breast tumours. Nature 406: 747-752.
Chan et al., (2009) Identification, molecular characterization, clinical prognosis, and therapeutic targeting of human bladder tumor-initiating cells. Proc Natl Acad Sci USA 106: 14016-14021.
Romano et al., (2009) An active role of the DeltaN isoform of p63 in regulating basal keratin genes K5 and K14 and directing epidermal cell fate. PLoS One 4: e5623.
Boldrup et al., (2007) DeltaNp63 isoforms regulate CD44 and keratins 4, 6, 14 and 19 in squamous cell carcinoma of head and neck. J Pathol 213: 384-391.
Breiman et al. Classification and Regression Trees, 1984, Monterey, Calif.: Wadsworth and Brooks
Koza, Genetic Programming: On the Programming of Computers by Means of natural Selection, 1992, Cambridge, Mass.: MIT Press.
Kalluri, et al., (2009) The basics of epithelial-mesenchymal transition. J Clin Invest 119, 1420-1428
McConkey, et al., (2009) Role of epithelial-to-mesenchymal transition (EMT) in drug sensitivity and metastasis in bladder cancer. Cancer Metastasis Rev 28, 335-344
Gregory, et al., (2008) The miR-200 family and miR-205 regulate epithelial to mesenchymal transition by targeting ZEB1 and SIP1. Nat Cell Biol 10, 593-601
Korpal, M., and Kang, Y. (2008) The emerging role of miR-200 family of microRNAs in epithelial-mesenchymal transition and cancer metastasis. RNA Biol 5, 115-119
Gregory, et al., (2008) MicroRNAs as regulators of epithelial-mesenchymal transition. Cell Cycle 7, 3112-3118
Adam, et al., (2009) miR-200 expression regulates epithelial-to-mesenchymal transition in bladder cancer cells and reverses resistance to epidermal growth factor receptor therapy. Clin Cancer Res 15, 5060-5072
Yang, et al., (1998) p63, a p53 homolog at 3q27-29, encodes multiple products with transactivating, death-inducing, and dominant-negative activities. Mol Cell 2, 305-316
Yang, et al., (1999) p63 is essential for regenerative proliferation in limb, craniofacial and epithelial development. Nature 398, 714-718
Westfall, M. D., and Pietenpol, J. A. (2004) p63: Molecular complexity in development and cancer. Carcinogenesis 25, 857-864
Karni-Schmidt, et al., (2011) Distinct expression profiles of p63 variants during urothelial development and bladder cancer progression. Am J Pathol 178, 1350-1360
Blanpain, C., and Fuchs, E. (2007) p63: revving up epithelial stem-cell potential. Nat Cell Biol 9, 731-733
Su, et al., (2010) TAp63 suppresses metastasis through coordinate regulation of Dicer and miRNAs. Nature 467, 986-990
Guo, et al., (2009) TAp63 induces senescence and suppresses tumorigenesis in vivo. Nat Cell Biol 11, 1451-1457
Yugawa, et al., (2010) DeltaNp63alpha repression of the Notch1 gene supports the proliferative capacity of normal human keratinocytes and cervical cancer cells. Cancer Res 70, 4034-4044
Keyes, et al., (2011) DeltaNp63alpha is an oncogene that targets chromatin remodeler Lsh to drive skin stem cell proliferation and tumorigenesis. Cell Stem Cell 8, 164-176
Rocco, et al., (2006) p63 mediates survival in squamous cell carcinoma by suppression of p73-dependent apoptosis. Cancer Cell 9, 45-56
Siegel, et al., Cancer statistics, 2012. CA Cancer J Clin 62, 10-29
Dinney, et al., (2004) Focus on bladder cancer. Cancer Cell 6, 111-116
Choi et al., (2012) p63 expression defines a lethal subset of muscle-invasive bladder cancers. PLoS One 7, e30206
Marquis, et al., (2012) p63 expression correlates with sensitivity to the Eg5 inhibitor ZD4877 in bladder cancer cells. Cancer Biol Ther 13
Chow, S et al., (2011) Wogonin induced apoptosis in human nasopharyngeal carcinoma cells by targeting GSK-3beta and DeltaNp63. Cancer Chemother Pharmacol 68, 835-845
Patrone, et al., (2000) Nuclear run-on assay using biotin labeling, magnetic bead capture and analysis by fluorescence-based RT-PCR. Biotechniques 29, 1012-1014, 1016-1017
Roman, et al., (2007) Downregulation of p63 upon exposure to bile salts and acid in normal and cancer esophageal cells in culture. Am J Physiol Gastrointest Liver Physiol 293, G45-53
Su, et al., (2009) TAp63 prevents premature aging by promoting adult stem cell maintenance. Cell Stem Cell 5, 64-75
Senoo, et al., (2007) p63 Is essential for the proliferative potential of stem cells in stratified epithelia. Cell 129, 523-536
Romano, R et al., (2009) An active role of the DeltaN isoform of p63 in regulating basal keratin genes K5 and K14 and directing epidermal cell fate. PLoS One 4, e5623
Shimomura, et al., (2008) P-cadherin is a p63 target gene with a crucial role in the developing human limb bud and hair follicle. Development 135, 743-753
Mani, et al., (2008) The epithelial-mesenchymal transition generates cells with properties of stem cells. Cell 133, 704-715
Stemmler, M. P. (2008) Cadherins in development and cancer. Mol Biosyst 4, 835-850
Rieger-Christ, et al., (2001) Expression of classic cadherins type I in urothelial neoplastic progression. Hum Pathol 32, 18-23
Jarrard, et al., (1997) P-Cadherin is a basal cell-specific epithelial marker that is not expressed in prostate cancer. Clin Cancer Res 3, 2121-2128
Lee, et al., (2006) The epithelial-mesenchymal transition: new insights in signaling, development, and disease. J Cell Biol 172, 973-981
Tsai, et al., Spatiotemporal Regulation of Epithelial-Mesenchymal Transition Is Essential for Squamous Cell Carcinoma Metastasis. Cancer Cell
Comijn, et al., (2001) The two-handed E box binding zinc finger protein SIP1 downregulates E-cadherin and induces invasion. Mol Cell 7, 1267-1278
Eger, et al., (2005) DeltaEF1 is a transcriptional repressor of E-cadherin and regulates epithelial plasticity in breast cancer cells. Oncogene 24, 2375-2385
Chang, et al., (2011) p53 regulates epithelial-mesenchymal transition and stem cell properties through modulating miRNAs. Nat Cell Biol 13, 317-323
Koga, et al., (2003) Impaired Delta Np63 expression associates with reduced beta-catenin and aggressive phenotypes of urothelial neoplasms. Br J Cancer 88, 740-747
Saini, et al., (2007) Genomic analysis of human microRNA transcripts. Proc Natl Acad Sci USA 104, 17719-17724
Rodriguez, et al., (2004) Identification of mammalian microRNA host genes and transcription units. Genome Res 14, 1902-1910
Baskerville, S., and Bartel, D. P. (2005) Microarray profiling of microRNAs reveals frequent coexpression with neighboring miRNAs and host genes. RNA 11, 241-247
Ozsolak, et al., (2008) Chromatin structure analyses identify miRNA promoters. Genes Dev 22, 3172-3183
Corcoran, et al., (2009) Features of mammalian microRNA promoters emerge from polymerase II chromatin immunoprecipitation data. PLoS One 4, e5279
el-Deiry, et al., (1992) Definition of a consensus binding site for p53. Nat Genet 1, 45-49
Oh, et al., DeltaNp63alpha protein triggers epithelial-mesenchymal transition and confers stem cell properties in normal human keratinocytes. J Biol Chem 286, 38757-38767
Piovan, et al., (2012) Oncosuppressive role of p53-induced miR-205 in triple negative breast cancer. Mol Oncol
Sabichi, et al., (2006) Characterization of a panel of cell lines derived from urothelial neoplasms: genetic alterations, growth in vivo and the relationship of adenoviral mediated gene transfer to coxsackie adenovirus receptor expression. J Urol 175, 1133-1137
Gandellini, et al., (2012) miR-205 regulates basement membrane deposition in human prostate: implications for cancer development. Cell Death Differ
Kurzrock, et al., (2008) Label-retaining cells of the bladder: candidate urothelial stem cells. Am J Physiol Renal Physiol 294, F1415-1421
Volkmer, et al., Three differentiation states risk-stratify bladder cancer into distinct subtypes. Proc Natl Acad Sci USA 109, 2078-2083
Polyak, K., and Weinberg, R. A. (2009) Transitions between epithelial and mesenchymal states: acquisition of malignant and stem cell traits. Nat Rev Cancer 9, 265-273
Chao, et al., (2010) Breast carcinoma cells re-express E-cadherin during mesenchymal to epithelial reverting transition. Mol Cancer 9, 179
Hugo, et al., (2007) Epithelial—mesenchymal and mesenchymal—epithelial transitions in carcinoma progression. J Cell Physiol 213, 374-383
Perou, C et al., (2000) Molecular portraits of human breast tumours. Nature 406, 747-752
Chan, et al., (2009) Identification, molecular characterization, clinical prognosis, and therapeutic targeting of human bladder tumor-initiating cells. Proc Natl Acad Sci USA 106, 14016-14021

1. A composition comprising an FGFR inhibitor for use in treating a to a patient determined to have a luminal bladder cancer comprising:
(a) an elevated expression level of one or more of the miR-200, MAL, FMO9P, BHMT, SNX31, KRT20, SPINK1, DHRS2, UPK2, UPK1A, VSIG2, CD24, CYP2J2, ERBB2, FABP4, FGRF3, FOXA1, GATA3, GPX2, KRT18, KRT19, KRT20, KRT7, KRT8, PPARG or XBP1 genes compared to a reference level;
(b) an elevated activation of AHR; estrogen receptor; MYC; SPDEF; Hdac; SMAD7; PPARA; TRIM24; PPARG; or SREBF2 compared to a reference level; or
(c) a decreased activation of TP53; STAT3; SMARCA4; PGR; NFkB; STAT1; HTT; SMAD3; SRF; or MKL1 compared to a reference level.

2. The composition of claim 1, wherein the patient was determined to have a luminal bladder cancer comprising an elevated expression level of one or more of CD24, CYP2J2, ERBB2, FABP4, FGRF3, FOXA1, GATA3, GPX2, KRT18, KRT19, KRT20, KRT7, KRT8, PPARG or XBP1 compared to a reference level.

3. The composition of claim 1, wherein the patient was determined to have a luminal bladder cancer comprising an elevated expression level of two, three, four or more of CD24, CYP2J2, ERBB2, FABP4, FGRF3, FOXA1, GATA3, GPX2, KRT18, KRT19, KRT20, KRT7, KRT8, PPARG or XBP1 compared to a reference level.

4. The composition of claim 1, wherein the patient was determined to have a luminal bladder cancer comprising an elevated expression level of miR-200 expression compared to a reference level.

5. The composition of claim 1, wherein the elevated level of miR-200 expression is at least 5-fold higher than the reference level.

6. The composition of claim 4, wherein the miR-200 is miR-200c.

7. The composition of claim 4, wherein the miR-200 is miR-200a, miR-200b, miR-141, or miR-429

8. The composition of claim 1, wherein the FGFR inhibitor is a selective FGFR3 inhibitor.

9. The composition of claim 1, wherein the FGFR inhibitor is PKC412; NF449; AZD4547; BGJ398; Dovitinib; TSU-68; BMS-582664; AP24534; PD173074; LY287445; ponatinib; or PD173073.

10. A method of treating a patient having bladder cancer, comprising administering an effective amount of an FGFR inhibitor to a patient determined to have a luminal bladder cancer comprising:
(a) an elevated expression level of one or more of the miR-200, MAL, FMO9P, BHMT, SNX31, KRT20, SPINK1, DHRS2, UPK2, UPK1A, VSIG2, CD24, CYP2J2, ERBB2, FABP4, FGRF3, FOXA1, GATA3, GPX2, KRT18, KRT19, KRT20, KRT7, KRT5, PPARG or XBP1 genes compared to a reference level;
(b) an elevated activation of AHR; estrogen receptor; MYC; SPDEF; Hdac; SMAD7; PPARA; TRIM24; PPARG; or SREBF2 compared to a reference level; or
(c) a decreased activation of TP53; STAT3; SMARCA4; PGR; NFkB; STAT1; HTT; SMAD3; SRF; or MKL1 compared to a reference level.

11. A composition comprising an anti-mitotic agent for use in treating a patient determined to have a basal bladder cancer comprising:
(a) an elevated expression level of one or more of the miR-205, CD44, CDH3, KRT1, KRT14, KRT16, KRT5, KRT6A, KRT6B, KRT6C, DSG3, KRT6B, LOC653499, LOC728910, PI3 or S100A7 genes compared to a reference level;
(b) an elevated activation of one or more of STAT3; NFkB; IRF7; JUN; STAT1; SP1; TP63; RELA; HIF1A; or IRF3 compared to a reference level; or
(c) a decreased activation of estrogen receptor; TRIM24; PPARA; Hdac; GATA3; N-cor; PIAS4; KLF2; SPDEF; or MEOX2 compared to a reference level.

12. The composition of claim 11, wherein the patient was determined to have a basal bladder cancer comprising an elevated expression level of one or more of miR-205, CD44, CDH3, KRT1, KRT14, KRT16, KRT5, KRT6A, KRT6B, or KRT6C compared to a reference level.

13. The composition of claim 12, wherein the patient was determined to have a basal bladder cancer comprising an elevated expression level of two, three, four or more of miR-205, CD44, CDH3, KRT1, KRT14, KRT16, KRT5, KRT6A, KRT6B, or KRT6C compared to a reference level.

14. The composition of claim 11, wherein the patient was determined to have a basal bladder cancer comprising an elevated expression level of miR-205 compared to a reference level.

15. The composition of claim 14, wherein the elevated level of miR-205 expression is at least 2-fold higher than the reference level.

16. The composition of claim 11, wherein the anti-mitotic agent comprises Paclitaxel, Docetaxel, Vinblastine, Vincristine, Vindesine, Vinorelbine, Colchicine, 1,3-diarylpropenone, AZD4877, epothilone B, or cisplatin.

17. The composition of claim 11, wherein the anti-mitotic agent comprises cisplatin.

18. A method of treating a patient having bladder cancer, comprising administering an effective amount of an anti-mitotic agent to a patient determined to have a basal bladder cancer comprising:
(a) an elevated expression level of one or more of miR-205, CD44, CDH3, KRT1, KRT14, KRT16, KRT5, KRT6A, KRT6B, KRT6C, DSG3, KRT6B, LOC653499, LOC728910, PI3 or S100A7 compared to a reference level;
(b) an elevated activation of one or more of STAT3; NFkB; IRF7; JUN; STAT1; SP1; TP63; RELA; HIF1A; or IRF3 compared to a reference level; or
(c) a decreased activation of estrogen receptor; TRIM24; PPARA; Hdac; GATA3; N-cor; PIAS4; KLF2; SPDEF; or MEOX2 compared to a reference level.

19. A composition comprising an anti-mitotic agent for use in treating a patient determined to have an immune infiltrating basal bladder cancer comprising an elevated expression level of one or more of the AIF1, BCL2, BTLA, CCLS, CD200R1, CD33, CD40, CD8B, CSF1, CTLA4, FASLG, FYB, FYN, HIVEP3, HLA-DRB6, ICAM3, IL10, IL12RB1, IL21R, L4I1, TNFSF14, TRAF1, TRAFD1, VAV1 or ZAP70 genes compared to a reference level.

20. The composition of claim 19, wherein the patient was determined to have an immune infiltrating basal bladder cancer comprising an elevated expression level of two, three, four or more of the AIF1, BCL2, BTLA, CCLS, CD200R1, CD33, CD40, CD8B, CSF1, CTLA4, FASLG, FYB, FYN, HIVEP3, HLA-DRB6, ICAM3, IL10, IL12RB1, IL21R, L4I1, TNFSF14, TRAF1, TRAFD1, VAV1 or ZAP70 genes compared to a reference level.

21. The composition of claim 19, wherein the anti-mitotic agent comprises Paclitaxel, Docetaxel, Vinblastine, Vincristine, Vindesine, Vinorelbine, Colchicine, 1,3-diarylpropenone, AZD4877, epothilone B, or cisplatin.

22. A composition comprising a non-cisplatin anticancer agent for use in treating a patient determined to have a p53-like bladder cancer comprising:
(a) an elevated expression level of one or more of ACTG2, CNN1, MYH11, MFAP4, PGM5, FLNC, ACTC1, DES, PCP4, or DMN compared to a reference level;
(b) an elevated activation of TP53; CDKN2A; RB1; MYOCD; MKL1; TCF3; SMARCB1; SRF; HTT; or Rb compared to a reference level;
(c) a decreased activation of TBX2; FOXM1; MYC; SMAD7; E2F2; MYCN; AHR; HEY2; NFE2L2; or SPDEF compared to a reference level; or
(d) an elevated or reduced expression level of one or more of the genes as indicated in Table C relative to a reference level.

23. The composition of claim 1, wherein the patient was determined to have a p53-like bladder cancer comprising:
(a) an elevated expression level of one or more of ACTG2, CNN1, MYH11, MFAP4, PGM5, FLNC, ACTC1, DES, PCP4, DMN compared to a reference level;
(b) an elevated activation of TP53; CDKN2A; RB1; MYOCD; MKL1; TCF3; SMARCB1; SRF; HTT; or Rb compared to a reference level; or
(c) a decreased activation of TBX2; FOXM1; MYC; SMAD7; E2F2; MYCN; AHR; HEY2; NFE2L2; or SPDEF compared to a reference level.

24. The composition of claim 22, wherein the patient was determined to have a bladder cancer comprising an elevated activation of TP53.

25. A method of treating a patient having bladder cancer, comprising administering an effective amount of a non-cisplatin anticancer therapy to a patient determined to have a bladder cancer comprising:
(a) an elevated expression level of one of the ACTG2, CNN1, MYH11, MFAP4, PGM5, FLNC, ACTC1, DES, PCP4 and DMN genes compared to a reference level;
(b) an elevated activation of TP53; CDKN2A; RB1; MYOCD; MKL1; TCF3; SMARCB1; SRF; HTT; or Rb compared to a reference level;
(c) a decreased activation of TBX2; FOXM1; MYC; SMAD7; E2F2; MYCN; AHR; HEY2; NFE2L2; or SPDEF compared to a reference level; or
(d) an elevated or reduced expression level of one or more of the genes as indicated in Table C relative to a reference level.

26. An in vitro method of characterizing a bladder cancer comprising obtaining a sample from a bladder cancer patient and testing to determine the level of expression or activation of a plurality of genes wherein:
(a) (i) an elevated expression level of one or more of the miR-200, MAL, FMO9P, BHMT, SNX31, KRT20, SPINK1, DHRS2, UPK2, UPK1A, VSIG2, CD24, CYP2J2, ERBB2, FABP4, FGRF3, FOXA1, GATA3, GPX2, KRT18, KRT19, KRT20, KRT7, KRT8, PPARG or XBP1 genes compared to a reference level; (ii) an elevated activation of AHR; estrogen receptor; MYC; SPDEF; Hdac; SMAD7; PPARA; TRIM24; PPARG; or SREBF2 compared to a reference level; or (iii) a decreased activation of TP53; STAT3; SMARCA4; PGR; NFkB; STAT1; HTT; SMAD3; SRF; or MKL1 compared to a reference level indicates that the patient has a luminal bladder cancer;
(b) (i) an elevated expression level of one or more of miR-205, CD44, CDH3, KRT1, KRT14, KRT16, KRT5, KRT6A, KRT6B, KRT6C, DSG3, KRT6B, LOC653499, LOC728910, PI3 or S100A7 compared to a reference level; (ii) an elevated activation of STAT3; NFkB; IRF7; JUN; STAT1; SP1; TP63; RELA; HIF1A; or IRF3 compared to a reference level; or (iii) a decreased activation of estrogen receptor; TRIM24; PPARA; Hdac; GATA3; N-cor; PIAS4; KLF2; SPDEF; or MEOX2 compared to a reference level indicates that the patient has a basal bladder cancer;
(c) (i) an elevated expression level of one of the genes in FIG. 6 (Cluster 2) compared to a reference level; (ii) an elevated activation of TP53; CDKN2A; RBI; MYOCD; MKL1; TCF3; SMARCB1; SRF; HTT; or Rb compared to a reference level; (iii) a decreased activation of TBX2; FOXM1; MYC; SMAD7; E2F2; MYCN; AHR; HEY2; NFE2L2; or SPDEF compared to a reference level; or (iv) an elevated or reduced expression level of one or more of the genes as indicated in Table C relative to a reference level indicates that the patient has a p53-like bladder cancer;
(d) an elevated expression level of one or more of the AIF1, BCL2, BTLA, CCLS, CD200R1, CD33, CD40, CD8B, CSF1, CTLA4, FASLG, FYB, FYN, HIVEP3, HLA-DRB6, ICAM3, IL10, IL12RB1, IL21R, L4I1, TNFSF14, TRAF1, TRAFD1, VAV1 or ZAP70 genes compared to a reference level indicates that the patient has an immune infiltrating basal bladder cancer; or
(e) an elevated or reduced expression level of one or more of the genes as indicated in Table D relative to a reference level indicates that the patient has a chemoresistant bladder cancer.

27. The method of claim 26, comprising testing to determine the level of expression or activation of a plurality of genes wherein:
(a) (i) an elevated expression level of one or more of the miR-200, MAL, FMO9P, BHMT, SNX31, KRT20, SPINK1, DHRS2, UPK2, UPK1A, VSIG2, CD24, CYP2J2, ERBB2, FABP4, FGRF3, FOXA1, GATA3, GPX2, KRT18, KRT19, KRT20, KRT7, KRT8, PPARG or XBP1 genes compared to a reference level; (ii) an elevated activation of AHR; estrogen receptor; MYC; SPDEF; Hdac; SMAD7; PPARA; TRIM24; PPARG; or SREBF2 compared to a reference level; or (iii) a decreased activation of TP53; STAT3; SMARCA4; PGR; NFkB; STAT1; HTT; SMAD3; SRF; or MKL1 compared to a reference level indicates that the patient has a luminal bladder cancer;
(b) (i) an elevated expression level of one or more of miR-205, CD44, CDH3, KRT1, KRT14, KRT16, KRT5, KRT6A, KRT6B, KRT6C, DSG3, KRT6B, LOC653499, LOC728910, PI3 or S100A7 compared to a reference level; (ii) an elevated activation of STAT3; NFkB; IRF7; JUN; STAT1; SP1; TP63; RELA; HIF1A; or IRF3 compared to a reference level; or (iii) a decreased activation of estrogen receptor; TRIM24; PPARA; Hdac; GATA3; N-cor; PIAS4; KLF2; SPDEF; or MEOX2 compared to a reference level indicates that the patient has a basal bladder cancer; or
(c) (i) an elevated expression level of one of the genes in FIG. 6 (Cluster 2) compared to a reference level; (ii) an elevated activation of TP53; CDKN2A; RBI; MYOCD; MKL1; TCF3; SMARCB1; SRF; HTT; or Rb compared to a reference level; (iii) a decreased activation of TBX2; FOXM1; MYC; SMAD7; E2F2; MYCN; AHR; HEY2; NFE2L2; or SPDEF compared to a reference level; or (iv) an elevated or reduced expression level of one or more of the genes as indicated in Table C relative to a reference level indicates that the patient has a p53-like bladder cancer.

28. The method of claim 26, further comprising testing to determine the level of expression or activation of 3, 4, 5, 6, 7, 8, 9 or 10 genes.

29. The method of claim 26, wherein the sample comprises a sample of the primary tumor, a circulating tumor cell, serum, or urine sample obtained from the patient.

30. The method of claim 26, wherein the level of expression in the sample is determined using Northern blotting, reverse transcription-quantitative real-time PCR (RT-qPCR), nuclease protection, an in situ hybridization assay, a chip-based expression platform, invader RNA assay platform, or b-DNA detection platform.

31. The method of claim 30, wherein the level of expression in the sample is determined using RT-qPCR.

32. The method of claim 26, further comprising identifying the bladder cancer patient as having a luminal bladder cancer, a basal bladder cancer, a p53-like bladder cancer, an immune infiltrating basal bladder cancer or a chemoresistant bladder cancer based on the testing.

33. The method of claim 32, wherein said identifying comprises providing a report.

34. The method of claim 33, wherein the report is a written or electronic report.

35. The method of claim 33, further comprising providing the report to the patient, a healthy care payer, a physician, and insurance agent, or an electronic system.

36. A method of treating a patient having bladder cancer, comprising:
(a) characterizing the bladder cancer in accordance with claim 26; and
(b) administering a therapy to the patient based on said characterizing.

37. The method of claim 36, further comprising administering:
(a) a FGFR inhibitor therapy to a patient having a luminal bladder cancer;
(b) an anti-mitotic therapy to a patient having a basal bladder cancer or an immune infiltrating basal bladder cancer; or
(c) a therapy that does not comprise cisplatin to a patient having a p53-activated bladder cancer.

38. An in vitro method of characterizing a bladder cancer comprising obtaining a sample from a bladder cancer patient and testing to determine the level of miR-200 or miR-205 in the sample relative to a reference level thereof, wherein an elevated level of miR-200 relative to the reference is indicative of the bladder cancer being a luminal bladder cancer and an elevated level of miR-205 relative to the reference is indicative of the bladder cancer being a luminal bladder cancer.

39. The method of claim 38, wherein the sample comprises a sample of the primary tumor or a circulating tumor cell, serum, or urine sample obtained from the patient.

40. The method of claim 38, wherein the level of miR-200 or miR-205 in the sample is determined using Northern blotting, reverse transcription-quantitative real-time PCR (RT-qPCR), nuclease protection, an in situ hybridization assay, a chip-based expression platform, invader RNA assay platform, or b-DNA detection platform.

41. The method of claim 40, wherein the level of miR-200 or miR-205 in the sample is determined using RT-qPCR.

42. The method of claim 38, further comprising identifying the bladder cancer patient as having a luminal bladder cancer if the miR-200 level is determined to be elevated relative to a reference level or a basal bladder cancer if the miR-205 level is determined to be elevated relative to a reference level.

43. The method of claim 42, wherein the elevated level of miR-200 is defined as an at least 5-fold higher level than the reference level.

44. The method of claim 42, wherein the elevated level of miR-205 is defined as an at least 2-fold higher level than the reference level.

45. The method of claim 42, wherein said identifying comprises providing a report.

46. The method of claim 45, wherein the report is a written or electronic report.

47. The method of claim 45, further comprising providing the report to the patient, a healthy care payer, a physician, and insurance agent, or an electronic system.

48. The method of claim 38, wherein the miR-200 is miR-200c.

49. The method of claim 38, wherein the miR-200 is miR-200a, miR-200b, miR-141, or miR-429.

50. An in vitro method of identifying a bladder cancer patient who is a candidate for FGFR inhibitor therapy comprising obtaining a sample from a bladder cancer patient and testing to determine the level of miR-200 in the sample relative to a reference level thereof, wherein an elevated level of miR-200 relative to the reference is indicative of the bladder cancer patient being a candidate for FGFR inhibitor therapy.

51. An in vitro method of identifying a bladder cancer patient who is a candidate for anti-mitotic therapy comprising obtaining a sample from a bladder cancer patient and testing to determine the level of miR-205 in the sample relative to a reference level thereof, wherein an elevated level of miR-205 relative to the reference is indicative of the bladder cancer patient being a candidate for anti-mitotic therapy.

52. An in vitro method of identifying an immune infiltrating bladder cancer in a patient comprising obtaining a sample from a bladder cancer patient and testing to determine the level of expression of one or more of the AIF1, BCL2, BTLA, CCLS, CD200R1, CD33, CD40, CD8B, CSF1, CTLA4, FASLG, FYB, FYN, HIVEP3, HLA-DRB6, ICAM3, IL10, IL12RB1, IL21R, L4I1, TNFSF14, TRAF1, TRAFD1, VAV1 or ZAP70 genes, wherein an elevated expression level of one or more of the AIF1, BCL2, BTLA, CCLS, CD200R1, CD33, CD40, CD8B, CSF1, CTLA4, FASLG, FYB, FYN, HIVEP3, HLA-DRB6, ICAM3, IL10, IL12RB1, IL21R, L4I1, TNFSF14, TRAF1, TRAFD1, VAV1 or ZAP70 genes compared to a reference level indicates that the patient has an immune infiltrating basal bladder cancer.

53. An in vitro method of identifying bladder cancer that has developed chemoresistance comprising obtaining a sample from a bladder cancer patient who has received at least a first chemotherapy and testing to determine the level of expression of one or more of the genes of Table D, wherein an elevated or reduced expression level of one or more of the genes as indicated in Table D relative to a reference level indicates that the patient has a chemoresistant bladder cancer.

54. A method treating a bladder cancer patient comprising:
(a) determining if the patient has developed a bladder cancer that is chemoresistant to a least a first chemotherapy in accordance with claim 54; and
(b) administering at least a second anti-cancer therapy to the patient.
